(19) United States
(12) Patent Application Publication
(10) Pub. No.: US 2004/0077020 A1
(43) Pub. Date: Apr. 22, 2004

(54) Diagnostic microarray for inflammatory bowel disease, crohn's disease and ulcerative colitis

(76) Inventors: Elizabeth E. Mannick, New Orleans, LA (US); Zhiyun Liu, Houston, TX (US); Maria-Stella Serrano, River Ridge, LA (US)

Correspondence Address: PATENT DEPARTMENT, TAYLOR, PORTER, BROOKS & PHILLIPS, L.L.P, P.O. BOX 2471, BATON ROUGE, LA, 70821-2471, US

(21) Appl. No.: 10/432,785

(22) PCT Filed: Nov. 30, 2001
(86) PCT No.: PCT/US01/45096

(51) Int. Cl. G01N 33/53

(52) U.S. Cl. 435/7.1

(57) ABSTRACT

Using RNA samples from mononuclear blood cells, gene sequences were identified that can be used to identify patients with IBD, and then distinguish patients with Crohn's disease from those with ulcerative colitis. Sequences were identified whose overexpression was distinct to patients with IBD, Crohn's disease, and ulcerative colitis when compared to patients with non-IBD intestinal disorders. Additionally, cluster analysis was used to identify twenty-five sequences that are IBD-related, and whose transcription pattern can be used in a microarray analysis to identify patients with IBD with a sensitivity of 84% and a specificity of 100%. Cluster analysis also identified thirty-six genes that could be used to distinguish patients with Crohn's disease from those with ulcerative colitis with a sensitivity of 89% and a specificity of 80%.

The benefit of the filing dates of provisional applications Nos. 60/286,602 filed Apr. 26, 2001, 60/264,909 filed Jan. 29, 2001, and 60/250,303 filed Nov. 30, 2000 are claimed under 35 U.S.C. § 119(e) in the United States, and are claimed under applicable treaties and conventions in all countries.

### TECHNICAL FIELD

This invention pertains to a method that uses DNA microarray hybridization to identify patients with inflammatory bowel disease, and to distinguish Crohn's disease from ulcerative colitis.

### BACKGROUND ART

Despite greater than fifty years of clinical experience with Crohn's disease and ulcerative colitis, collectively known as inflammatory bowel disease (IBD), the precise etiology of these diseases remains unknown and the morbidity they engender high. Further, between 10-15% of patients cannot be accurately diagnosed as having either Crohn's disease or ulcerative colitis and are classified as indeterminate colitis, making treatment decisions and evaluation of long-term prognosis difficult.

IBD susceptibility loci have recently been mapped to chromosomes 1, 3, 4, 6, 10, 12, 16, X and 22. See J. P. Hugot et al., “Genome-wide scanning in inflammatory bowel diseases,” Dig. Dis., vol. 16, pp. 364-369 (1998); J. Hampe et al., “A genomewide analysis provides evidence for novel linkages in inflammatory bowel disease in a large European cohort,” Am. J. Hum. Genet., vol. 64, pp. 808-816 (1999); and K. G. Becker et al., “Clustering of non-major histocompatibility complex susceptibility candidate loci in human autoimmune diseases,” Proc. Natl. Acad. Sci. USA, vol. 95, pp. 9979-9984 (1998). Due to the painstaking nature of linkage analysis and positional cloning techniques, no IBD candidate gene has as yet been identified.

The current revolution in molecular genetics offers new hope of identifying genes that may play a role in disease susceptibility, etiology, and diagnosis. Microarray gene analysis has recently been added to the arsenal of molecular genetic techniques. See M. Schena et al., “Qualitative monitoring of gene expression patterns with a complementary DNA microarray,” Science, vol. 270, pp. 467-470 (1995). Microarray analysis allows an investigator to screen for thousands of genes in a relatively small patient sample such as a single endoscopic biopsy or a small amount of blood (<2 cc). A microarray is a glass slide, microchip, or membrane with cDNA of thousands of known sequences spotted on it. These microarrays then serve as sequence targets for hybridization to cDNA probes prepared from RNA samples from cells or tissues. A two-color fluorescence labeling technique is generally used in the preparation of the cDNA probes such that a simultaneous hybridization, but separate detection of signals, provides a comparative analysis and a determination of the relative abundance of specific genes expressed. Microarrays can be constructed from specific cDNA clones of interest, a cDNA library, or a select number of open-reading frames from a genome sequencing database to allow a large-scale functional analysis of expressed sequences. See R. A. Heller et al., “Discovery and analysis of inflammatory disease-related genes using cDNA microarrays,” Proc.Natl.Acad.Sci.USA, vol. 94, pp. 2150-2155 (1997); and M. Schena et al., “Quantitative Monitoring of Gene Expression Patterns with a Complementary DNA Microarray,” Science, vol. 270, pp. 467-470 (1995). An advantage of microarray technology is that more than one oligonucleotide sequence per gene may be included on the array, potentially providing greater specificity.

Microarray analysis offers a complementary approach to gene mapping strategies. However, the diagnostic genes identified by microarray analysis may be different from the genes found by positional cloning, because the occurrence of RNA in the sample used for the microarray analysis may be influenced by environmental factors, including medical treatments, that induce or suppress genetic expression.

Microarray analysis using selected human sequences of probable significance in inflammation, as well as with sequences expressed in peripheral human blood cells, has been used to compare gene expression in tissue samples of rheumatoid arthritis (late stage rheumatoid synovial tissue) and inflammatory bowel disease (inflamed lower intestinal mucosa of patients with Crobn's disease). See Heller et al., 1997.

Samples from the mucosal intestinal wall of ulcerative colitis and Crohn's disease patients with inflamed and noninflamed controls have been used in a microarray analysis of approximately 6000 sequences (Affymetrix GeneChip). See B. K. Dieckgraefe et al., “Analysis of mucosal gene expression in inflammatory bowel disease by parallel oligonucleotide arrays,” Physiol. Genomics, vol. 4, pp. 1-11 (2000); I. Lawrence, C. Fiocchi, S. Chakravarti, “Ulcerative colitis and Crohn's disease: distinctive gene expression profiles and novel susceptibility candidate genes.” Hum Mol Genet 2001;10:445-56; and International Application No. WO 01/29269A2.

### DISCLOSURE OF INVENTION

We have discovered, using RNA samples from mononuclear blood cells, gene sequences that can be used to identify patients with IBD, Crohn's disease, and ulcerative colitis. Sequences were identified whose over- or underexpression was distinct to patients with IBD, Crohn's disease, or ulcerative colitis when compared to patients with non-IBD intestinal disorders. Additionally, cluster analysis was used to identify twenty-five sequences that are IBD-related, and whose transcription pattern can be used in a microarray analysis to identify patients with IBD with a sensitivity of 84% and a specificity of 100%. Cluster analysis also identified thirty-six genes that could be used to distinguish patients with Crohn's disease from those with ulcerative colitis with a sensitivity of 80% and a specificity of 89%.

### MODES FOR CARRYING OUT THE INVENTION

The microarray information on over- and under-expression of gene sequences can be used to develop diagnostic tests for Crohn's disease, ulcerative colitis and inflammatory bowel disease. It can be used to identify subcategories of patients in order to predict disease prognosis. For example, Crohn's disease is subclassifed into fibrostenosing and inflammatory subtypes, which may correspond to genotypic as well as phenotypic differences. It can also be used to monitor the results of drug therapy for inflammatory bowel disease, ulcerative colitis and Crohn's disease. Finally, it has the potential to be used to identity drug targets in order to develop new therapies for the treatment of inflammatory bowel disease. For example, an overexpressed gene that is secreted and/or membrane-bound, if overexpressed at the protein level, could be targeted with a monoclonal antibody. Two genes identified here that are overexpressed in patients with inflammatory bowel disease, antigen CD36 (clone 13), and APRIL protein, might serve as targets for monoclonal antibody therapy. Alternatively, underexpressed genes, such as G protein-gamma 4, ARC34, SBPS-1 or carbonic anhydrase II, which, based on their biological function, could play a role in IBD pathogenesis, could be targets for replacement with gene therapy. For a review of the use of microarray data for prognostic, pharmacotherapy monitoring and drug discovery uses, see S. Braxton et al., “The integration of microarray information in the drug development process,” Curr. Opin. Biotechnol., vol. 9, pp. 643-649 (1988); and G. I. Adam et al., “Pharmacogenomics to predict drug response,” Pharmacogenomics, vol. 1, pp. 5-14 (2000); and S. F. Grant, “Pharmacogenetics and pharmacogenomics: tailored drug therapy for the 21st century,” Trends Pharmacol. Sci., vol. 22, pp. 3-4 ( 2001).

### EXAMPLE 1

### Identification of Overexpressed and Underexpressed Genes in Patients With Crohn's Disease

A genetic study was completed using microarray analysis to compare the expression of 2400 sequences in peripheral blood mononuclear cells (“TBMC”) from 14 patients with Crohn's disease as compared to 10 healthy, age-matched controls. A MICROMAX™ Human cDNA Microarray System was obtained from NEN Life Sciences, Inc. (Boston, Mass.). The MICROMAX™ system comes with 2400 human sequences spotted on a glass slide. Except for a small number of plant control sequences, all sequences are from over 50 human cDNA libraries representing more than 10 tissue sources created by Alphagene, Incorporated (Woburn, Mass.). The list of sequences included on the microarray slide is available on the website for NEN Life Sciences, Inc., http://www.nen.com/products/gene-list5.txt. This system allows direct, quantitative comparison of gene expression between a test (e.g., patient) sample labeled with, for example, a red fluorescent dye and a control sample labeled with, for example, a green fluorescent dye, both hybridized on the same microarray slide. The ratio of red-to-green fluorescent intensity at each site represents the ratio of differential gene expression (after normalization for housekeeping genes and subtraction of background). Advantages of this system are the ability to hybridize samples from two groups under the same conditions, as well as the easy visualization of the results of the analysis.

Peripheral blood mononuclear cells (PBMC) were extracted from blood drawn from each patient and controls using Ficoll (Sigma, St. Louis, Mo.). Although ideally PBMC should be sorted by cell type (T cells, B cells, monocytes, dendritic cells, etc.), the amount of total extracted RNA was already at the lower limit for reliable detection. Follow-up flow cytometry experiments, will be conducted for genes of particular interest to identify the exact cellular sources of the over- or under-expressed genes.

RNA was extracted from PBMC using the Trisol method. Extracted RNA was purified using a phenol-chloroform extraction. See F. Ausubel et al., Short Protocols in Molecular Biology, 3rd Edition, Wiley & Company, pp. 2-3 (1995). RNA was measured by spectrophotometry, and 1.5 mg of RNA was run on a 1% agarose gel to assess RNA quality. RNA was converted to cDNA using reverse transcriptase (NEN Life Sciences, Inc.) in a thermocycler. During this step, cDNA of the patient samples was labeled with dinitrophenol (DNP), and cDNA of the control samples with biotin. cDNA of each sample was then purified by ethanol precipitation, analyzed by dot blot using horseradish peroxidase (HRP). HRP-conjugated, anti-DNP antibody and streptavidin-HRP conjugate, and the total amount was estimated using known cDNA standards. All of the above procedures followed the detailed protocol of and used the reagents provided with the MICROMAX™ kit available from NEN Life Sciences, Inc.

Equal amounts of cDNA from a patient and from an age- and sex-matched control were combined and denatured in hybridization buffer (NEN Life Sciences, Inc.) at 90° C., and then hybridized on the glass slide microarray (NEN Life Sciences, Inc.) at 65° C. overnight. After washing in a sodium citrate-sodium chloride buffer (3M sodium chloride, 0.3 m sodium citrate, ph 7.0) (NEN Life Sciences, Inc.) three times, the slides were blocked with normal goat serum, incubated with anti-DNP-HRP conjugate, followed by Cyanine 3 (red fluorescent) dye conjugated to tyramide for signal amplification. After rinsing and HRP inactivation, the slides were incubated with streptavidin-HRP conjugate, followed by Cyanine 5 (green fluorescent) dye conjugated to tyramide and rinsed. All of the above reagents were provided in the MICROMAX™ kit. After drying, slides were sent to NEN Life Sciences for laser scanning. A ratio of red-to-green (patient-to-control) fluorescent intensities was calculated after subtracting background intensity from the signal and normalizing for housekeeping sequences in the array.

The 14 patients ranged in age from 10 to 55; 6 had been treated for Crohn's disease and 8 were untreated, 10 were males and 4 female, 11 had active disease and 3 were in remission. Table 1 is a list of the 25 sequences of the 2400 sequences assayed that were the most overexpressed and of the 25 sequences that were the most underexpressed in the fourteen Crohn's patients. The sequences are sorted by the median ratio after normalization of data by median centering each array. A median ratio of greater than 2 or less than 0.50 is often used to identify sequences of potential biological significance; i.e., genes that are overexpressed or underexpressed, respectively.

TABLE 1
Gene Expression in Fourteen Patients with Crohn's Disease

| | |
|---|---|
| Sequence ID with Genbank Accession # in Parentheses | CD Median |

Overexpressed Genes in Crohn's Disease

| | |
|---|---|
| Messenger RNA for beta-globin (Acc# M34539) | 3.41 |
| mRNA for brain acyl-CoA hydrolase (Acc# D88894) | 1.91 |
| Breast epithelial antigen BA46 (Acc# U58516) | 1.83 |
| Ribosomal protein (Acc# M15661) | 1.83 |
| MOP4 (Acc# U51625) | 1.79 |
| Tristetraproline (TTP) (Acc# M63625) | 1.79 |
| atrophin-1 related protein (Acc# AF001845) | 1.78 |
| mRNA for hepatoma-derived growth factor (Acc# D16431) | 1.78 |
| Fatty acid binding protein homologue (PA-FABP) (Acc# M94856) | 1.75 |
| Tumorous imaginal discs protein Tid56 homolog (TID1) (Acc# AF061749) | 1.75 |
| Cellular growth-regulating protein (Acc# L10844) | 1.75 |
| Armadillo repeat protein (Acc# U51269). | 1.73 |
| Messenger RNA for alpha globin (Acc# V00493) | 1.72 |
| Protein tyrosine phosphatase PTPCAAX1 (hPTPCAAX1) (Acc# U48296) | 1.68 |
| Nicotinic acetylcholine receptor alpha3 subunit precursor (Acc# U6243) | 1.67 |
| mRNA for brain cholecystokinin receptor (Acc# D13305) | 1.66 |
| Hom. of Drosophila splicing regulator suppressor-of-white-apricot (Acc# U08377) | 1.65 |
| Muscle glycogen synthase (Acc# J04501) | 1.64 |
| mRNA for MAT8 protein (Acc# X93036) | 1.62 |
| jun-B mRNA for JUN-B protein (Acc# X51345) | 1.62 |
| Acidic calponin (Acc# S80562) | 1.61 |
| Type 1 neurofibromatosis protein (Acc# M82814) | 1.61 |
| mRNA for proton-ATPase-like protein (Acc# D89052) | 1.60 |
| Ribosomal protein S20 (RPS20) (Acc# L06498) | 1.60 |
| MRNA for S100 alpha protein (Acc# X58079) | 1.58 |

Underexpressed Genes in Crohn's Disease

| | |
|---|---|
| Adult skeletal muscle alpha-actin mRNA (Acc# J00068) | 0.17 |
| mRNA for vascular smooth muscle alpha-actin (Acc# X13839) | 0.21 |
| mRNA for enteric smooth muscle gamma-actin (Acc# X16940) | 0.22 |
| MHC class I HLA-C-alpha-2 chain and alternative, clones 4 and 10 (Acc# M24097) | 0.23 |
| mRNA for beta-actin (Acc# AB004047) | 0.23 |
| mRNA for HLA-Cw*0704 (Acc# X83394) | 0.23 |
| ACTB mRNA for mutant beta-actin (beta'-actin) (Acc# X63432) | 0.25 |
| Wnt-13 mRNA (Acc# Z71621) | 0.25 |
| mRNA for immunoglobulin kappa light chain (Acc# Y14736) | 0.28 |
| mRNA for HLA class-I (HLA-A26) heavy chain (clone cMIY-2) (Acc# D32130) | 0.29 |
| mRNA for HLA-DR antigens associated invariant chain (p33) (Acc# X00497) | 0.29 |
| MHC HLA-B39 (Acc# L42024) | 0.30 |
| Class II histocompatibility antigen beta-chain (pII-beta-3) (Acc# X00699) | 0.30 |
| mRNA for seryl-tRNA synthetase (Acc# X91257) | 0.31 |
| mRNA for KIAA0391 gene (Acc# AB002389) | 0.32 |
| mRNA for cytoskeletal gamma-actin (Acc# X04098) | 0.34 |
| Methionine aminopeptidase (Acc# U29607) | 0.37 |
| Epidermal growth factor receptor substrate (eps15) (Acc# U07707) | 0.38 |
| mRNA for ribosomal protein L3 (Acc# X73460) | 0.38 |
| Brain-expressed HHCPA78 homolog (Acc# X73591) | 0.39 |
| Wilm's tumor-related protein (QM) (Acc# M64241) | 0.40 |
| mRNA for B120 (Acc# AB001895) | 0.40 |
| mRNA for RAB7 protein (Acc# X93499) | 0.40 |
| Sigma receptor (Acc# U75283) | 0.41 |
| mRNA for ribosomal protein L14 (Acc# D87735) | 0.41 |

### EXAMPLE 2

### Identification of Overexpressed and Underexpressed Genes in Patients With Ulcerative Colitis

A second genetic study was completed using microarray analysis to compare the expression of 2400 sequences in peripheral blood mononuclear cells (“TBMC”) from 11 patients with ulcerative colitis as compared to 10 healthy, age-matched controls. Of the 11 patients with ulcerative colitis, 5 had active disease and 6 were inactive. Six were untreated, and five were being treated with some combination of steroids, immunosuppressive agents, and 5-ASA compounds. The procedure was as described in Example 1, using the MICROMAX™ Human cDNA Microarray System obtained from NEN Life Sciences, Inc. (Boston, Mass.). Table 2 is a list of the 25 sequences of the 2400 sequences assayed that were the most overexpressed and of the 25 sequences that were the most underexpressed in the eleven UC patients. The sequences are sorted by the median ratio after normalization of data by median centering each array. A median ratio of greater than 2 or less than 0.50 is often used to identify genes of potential biological significance.

TABLE 2
Gene Expression in Ulcerative Colitis Patients

| | |
|---|---|
| Sequence ID with Genbank Accession # in Parentheses | CD Median |

Overexpressed Genes in Ulcerative Colitis

| | |
|---|---|
| Ribosomal protein S25 (Acc# M64716) | 4.22 |
| alpha-CP1 (Acc# U24223) | 4.09 |
| CL 100 mRNA for protein tyrosine phosphatase (Acc# X68277) | 4.05 |
| Lysosomal-associated multitransmembrane protein (Acc# U51240) | 3.62 |
| mRNA for brain acyl-CoA hydrolase (Acc# D88894) | 3.44 |
| Ubiquitin-52 amino acid fusion protein (Acc# X56999) | 3.37 |
| mRNA for insulinoma pre-proinsulin (Acc# X70508) | 3.37 |
| Lymph node homing receptor (Acc# M25280) | 3.35 |
| Ribosomal protein S20 (RPS20) (Acc# L06498) | 3.20 |
| mRNA for Clock (Acc# AB005535) | 3.15 |
| N-formylpeptide receptor (fMLP-R26) (Acc# M60627) | 2.99 |
| Palmitoyl protein thioesterase (Acc# U44722) | 2.98 |
| U2 small nuclear RNA-associated B″ antigen (Acc# M15841) | 2.93 |
| Putative tumor suppressor (LUCA15) (Acc# U23946) | 2.89 |
| BCL2/adenovirus E1B 19 kD-interacting protein 2 (Acc# U15173) | 2.88 |
| X box binding protein-1 (XBP-1) (Acc# M31627) | 2.86 |
| mRNA for ribosomal protein L31 (Acc# X15940) | 2.84 |
| mRNA for ribosomal protein L26 (Acc# X69392) | 2.84 |
| Ribosomal protein L18 (RPL18) (Acc# L11566) | 2.79 |
| H5; and platelet glycoprotein Ib beta chain (Acc# U59632) | 2.78 |
| COX VIIc gene for subunit VIIc of cytochrome c oxidase (Acc# U53328) | 2.77 |
| 14-3-3 protein epsilon isoform (Acc# U43399) | 2.74 |
| cAMP-dependent protein kinase subunit RII-beta (Acc# M31158) | 2.73 |
| rab11a GTPase (Acc# AF000231) | 2.72 |
| mRNA for slow skeletal troponin C (TnC) (Acc# X07897) | 2.70 |

Underexpressed Genes in Ulcerative Colitis

| | |
|---|---|
| Tumor necrosis factor superfamily member LIGHT(Acc# F036581) | 0.15 |
| Nicotinic acetylcholine receptor alpha3 subunit precursor (Acc# U62432) | 0.15 |
| p300 protein (Acc# U01877) | 0.16 |
| Endogenous retrovirus type C oncovirus sequence (Acc# L77964) | 0.17 |
| Macrophage colony-stimulating factor (M-CSF1) (Acc# M27087) | 0.19 |
| Adult skeletal muscle alpha-actin mRNA (Acc# J00068) | 0.20 |
| Caco-2 oligopeptide transporter (PEPT1) (Acc# AF043233) | 0.20 |
| Carcinoembryonic antigen mRNA (CEA) (Acc# M29540) | 0.22 |
| Interleukin 1 receptor (Acc# M27492) | 0.23 |
| mRNA for enteric smooth muscle gamma-actin (Acc# X16940) | 0.26 |
| FGFR2 mRNA (Acc# Z79129) | 0.26 |
| Keratin 8 mRNA, 5′ end (Acc# M26512) | 0.26 |
| p167 (Acc# U58046) | 0.27 |
| mRNA for KIAA0331 gene (Acc# AB002329) | 0.27 |
| mRNA for cardiac myosin binding protein-C (Acc# X84075) | 0.27 |
| mRNA for KIAA0355 gene (Acc# AB002353) | 0.28 |
| Methionine aminopeptidase (Acc# U29607) | 0.28 |
| Synaptotagmin (Acc# M55047) | 0.29 |
| RBQ-3 mRNA (Acc# X85134) | 0.29 |
| Leukocyte-associated Ig-like receptor-1 (LAIR-1) (Acc# AF013249) | 0.30 |
| TLS, translocated in liposarcoma (Acc# S62140) | 0.31 |
| G protein-coupled receptor (STRL22) (Acc# U68030) | 0.32 |
| mRNA for Sec23B isoform, 2450bp (Acc# X97065) | 0.33 |
| Xeroderma pigmentosum UV-damaged DNA binding factor (Acc# U32986) | 0.33 |
| Retroviral receptor mRNA (Acc# X59155) | 0.34 |

### EXAMPLE 3

### Identification of Genes Whose Expression Indicates LOD, Crohn's Disease, or Ulcerative Colitis

Another microarray study was performed to compare gene expression in peripheral blood mononuclear cells from 14 patients with Crohn's, 11 patients with ulcerative colitis, and 10 patients with chronic intestinal inflammation unrelated to inflammatory bowel disease (Clostridium difficile colitis, celiac disease, Helicobacter pylon gastritis, food poisoning, disseminated Mycobacterial infection with gastrointestinal involvement, disseminated Histoplasmosis with gastrointestinal involvement, sarcoidosis with gastrointestinal involvement, colonic adenomatosis with diverticulosis, eosinophilic gastroenteritis/colitis, irritable bowel syndrome) (“non-IBD disease patients”), and 35 age- and sex-matched unaffected controls. This study used the same IBD patients as in Examples 1 and 2. This study was designed to identify specific genes whose expression pattern might be used to diagnose patients with inflammatory bowel disease (“IBD”), and also to distinguish patients with Crohn's disease from those with ulcerative colitis. The RNA extraction and the microarray analysis were performed as described above in Example 1 using the MICROMAX™ Human cDNA Microarray System from NEN Life Sciences, Inc. (Boston, Mass.).

In order to identify IBD-specific genes, the following criteria were applied to the results obtained from scanning the microarray slides. Sequences were identified as IBD-related genes if the gene was overexpressed or underexpressed by a factor of two or more in at least 25% of the patients with either ulcerative colitis or Crohn's disease as compared to patients with chronic intestinal inflammation unrelated to IBD after normalization by median centering arrays. The genes listed in Table 3 met this criteria.

Table 3 also lists sequences specific for Crohn's disease and for ulcerative colitis (UC). Sequences were identified as Crohn's-specific genes if the gene was overexpressed or underexpressed by a factor of 2 or more in at least 25% of patients with Crohn's disease as compared to patients with chronic intestinal inflammation unrelated to IBD.

Sequences were identified as UC-specific genes if the gene was overexpressed or underexpressed by a factor of 2 or more in at least 25% of patients with ulcerative colitis as compared to patients with other inflammatory gastrointestinal disorders.

TABLE 3
IBD-Related Genes and Percent of Patients Over/Underexpressing

| | | | | |
|---|---|---|---|---|
| | | | | Number |
| | | Non-IBD | Number | Patients with |
| | Disease | Other GI | Patients with | non-IBD Other |
| Sequence ID with GenBank Accession Number | Median | Median | Disease (%) | GI (%) |

A. Sequences Identified for IBD
Overexpressed Genes

| | | | | |
|---|---|---|---|---|
| Acidic Calponin (Acc# S80562) | 2.04 | 0.92 | 13/25 (52) | 1/10 (10) |
| GPx-4 mRNA (Acc# X71973) | 0.94 | 1.14 | 10/25 (40) | 0/10 (0) |
| mRNA for serine/threonine protein kinase | 1.55 | 1.20 | 10/25 (40) | 0/10 (0) |
| (Acc# D86550) | | | | |
| Glial fibrillary acidic protein (GFAP) | 1.32 | 0.93 | 9/25 (36) | 0/10 (0) |
| (Acc# J04569) | | | | |
| Tumor susceptibility protein (TSG101) | 1.39 | 1.27 | 9/25 (36) | 0/10 (0) |
| (Acc# U82130) | | | | |
| Beta-sarcoglycan A3b (Acc# U31116) | 1.21 | 1.03 | 9/25 (36) | 0/10 (0) |
| Rab11a GTPase (Acc# AF000231) | 2.03 | 1.14 | 13/25 (52) | 2/10 (20) |
| Autosomal dominant polycystic kidney disease 1 | 1.10 | 1.39 | 8/25 (32) | 0/10 (0) |
| (Acc# U24497) | | | | |
| mRNA for ZIP-kinase (Acc# AB007144) | 1.12 | 0.66 | 8/25 (32) | 0/10 (0) |
| mRNA for skeletal muscle 190 kD protein | 1.42 | 1.11 | 8/25 (32) | 0/10 (0) |
| (Acc# X69090) | | | | |
| mRNA for KIAA0168 (Acc# D79990) | 1.14 | 0.81 | 8/25 (32) | 0/10 (0) |
| mRNA for Insulinoma pre-proinsulin (Acc# | 2.07 | 1.32 | 13/25 (52) | 2/10 (20) |
| X70508) | | | | |
| Replication protein A 32-kDa subunit (Acc# | 1.43 | 1.28 | 10/25 (40) | 1/10 (10) |
| J05249) | | | | |
| mRNA for G13 protein (Acc# X98054) | 1.54 | 1.38 | 10/25 (40) | 1/10 (10) |
| mRNA for APRIL (Acc# Y07969) | 1.33 | 1.26 | 7/25 (28) | 0/10 (0) |
| Lipoxygenase (Acc# J03600) | 1.00 | 0.95 | 7/25 (28) | 0/10 (0) |
| Excitatory amino acid transporter 4 (Acc# | 1.23 | 1.05 | 7/25 (28) | 0/10 (0) |
| U18244) | | | | |
| D1g3 (Acc# J03068) | 1.07 | 1.07 | 7/25 (28) | 0/10 (0) |
| Zinc finger protein 216 splice variant 2 | 1.03 | 1.06 | 7/25 (28) | 0/10 (0) |
| (ZNF216) (Acc# AF06234) | | | | |
| SCG10, neuron-specific growth-associated | 1.13 | 0.78 | 7/25 (28) | 0/10 (0) |
| protein (Acc# S82024) | | | | |
| Protein tyrosine kinase (Acc# U02680) | 1.09 | 0.92 | 7/25 (28) | 0/10 (0) |
| PROS-27 (Acc# X59417) | 1.07 | 0.84 | 7/25 (28) | 0/10 (0) |
| Pancreatic lipase related protein-1 (PLRP1) | 1.18 | 0.97 | 7/25 (28) | 0/10 (0) |
| (Acc# M93283) | | | | |
| Arginosuccinate lyase (Acc# J03058) | 1.17 | 1.04 | 7/25 (28) | 0/10 (0) |
| mRNA for Mr 110,000 antigen (Acc# | 1.24 | 1.12 | 7/25 (28) | 0/10 (0) |
| D64154) | | | | |
| mRNA for IgG Fc binding protein (Acc# | 0.98 | 1.06 | 7/25 (28) | 0/10 (0) |
| D84239) | | | | |
| mRNA encoding IMP: pyrophosphate | 1.21 | 0.91 | 12/25 (48) | 2/10 (20) |
| phophoribosyltransferase (Acc# V00530) | | | | |
| Deubiquitinating enzyme UnpEL (UNP) | 1.10 | 1.12 | 12/25 (48) | 2/10 (20) |
| (Acc# U37707) | | | | |
| Glutathione-S-transferase homolog (Acc# | 2.30 | 1.48 | 13/25 (56) | 3/10 (30) |
| U90313) | | | | |
| (p23) (Acc# L24804) | 1.38 | 1.06 | 9/25 (36) | 1/10 (10) |
| Serine/threonine kinase 11 (STK11) | 1.47 | 1.22 | 9/25 (36) | 1/10 (10) |
| (Acc# AF035625) | | | | |
| RSU-1/RSP-1 (Acc# L12535) | 1.43 | 1.11 | 9/25 (36) | 1/10 (10) |
| Alpha-2 collagen type VI-a′ gene, exon 1 | 1.20 | 0.82 | 9/25 (36) | 1/10 (10) |
| (Acc# M34573) | | | | |
| mRNA for Hrs. (Acc# D84064) | 1.41 | 0.99 | 9/25 (36) | 1/10 (10) |
| mRNA for cytosolic asparaginyl-tRNA | 1.17 | 0.95 | 9/25 (36) | 1/10 (10) |
| synthetase (Acc# AJ000334) | | | | |
| mRNA for carboxyl methyltransferase (Acc# | 1.58 | 1.17 | 9/25 (36) | 1/10 (0) |
| D13892) | | | | |

Underexpressed Genes

| | | | | |
|---|---|---|---|---|
| Methionine aminopeptidase (Acc# U29607) | 0.28 | 0.90 | 16/25 (64) | 1/10 (10) |
| G protein gamma-4 subunit (Acc# U31382) | 0.56 | 0.99 | 12/25 (48) | 0/10 (0) |
| hCDC10, CDC10 homolog (Acc# S72008) | 0.66 | 0.73 | 10/25 (40) | 0/10 (0) |
| mRNA for C3G protein (Acc# D21239) | 0.82 | 1.01 | 10/25 (40) | 0/10 (0) |
| Sorting nexin 2 (SNX2) (Acc# AF043453) | 0.62 | 1.12 | 10/25 (40) | 0/10 (0) |
| Retroviral receptor mRNA (Acc# X59155) | 0.58 | 0.94 | 12/25 (48) | 1/10 (10) |
| Achaete scute homologous protein (ASH1) (Acc# | 0.77 | 0.70 | 9/25 (36) | 9/25 (36) |
| L08424) | | | | |
| Carbonic anhydrase II (Acc# J03037) | 0.78 | 1.28 | 9/25 (36) | 0/10 (0) |
| mRNA for KIAA0210 (Acc# D86965) | 0.95 | 0.92 | 9/25 (36) | 0/10 (0) |
| mRNA for maleylacetoacetate isomerase (Acc# | 0.68 | 1.13 | 9/25 (36) | 0/10 (0) |
| AJ001838) | | | | |
| mRNA for SHPS-1 (Acc# D86043) | 0.73 | 1.49 | 9/25 (36) | 0/10 (0) |
| PKC alpha mRNA for protein kinase C alpha | 0.62 | 0.88 | 9/25 (36) | 0/10 (0) |
| (Acc# X52479) | | | | |
| Sodium-hydrogen exchanger 6 (NHE-6) (Acc# | 0.70 | 0.84 | 9/25 (36) | 0/10 (0) |
| AF030409) | | | | |
| Caco-2 oligopeptide transporter (PEPT1) (Acc# | 0.43 | 0.58 | 16/25 (64) | 3/10 (30) |
| AF043233) | | | | |
| 26S protease subunit 5b, 50 kda subunit (Acc# | 0.57 | 0.88 | 11/25 (44) | 1/10 (10) |
| S79862) | | | | |
| mRNA for protein kinase, PKX1 (Acc# X85545) | 0.67 | 1.16 | 11/25 (44) | 1/10 (10) |
| D53 (hD53) | 1.09 | 1.49 | 8/25 (32) | 0/10 (0) |
| mRNA for DCRA (Acc# D87343) | 0.77 | 0.85 | 8/25 (32) | 0/10 (0) |
| mRNA for mosaic protein LR11 (Acc# Y08110) | 0.90 | 1.45 | 8/25 (32) | 0/10 (0) |
| mRNA for ORF, Xq terminal protein (Acc# | 0.82 | 1.27 | 8/25 (32) | 0/10 (0) |
| D16469) | | | | |
| Rac3 (RAC3) (Acc# AF008591) | 0.88 | 0.99 | 8/25 (32) | 0/10 (0) |
| Sorbitol dehydrogenase (Acc# U07361) | 0.81 | 1.38 | 8/25 (32) | 0/10 (0) |
| SREBP-1 (Acc# U00968) | 0.48 | 1.30 | 13/25 (52) | 2/10 (20) |
| Translation repressor NAT1 (Acc# U76111) | 0.85 | 1.03 | 8/25 (32) | 0/10 (0) |
| ADP-ribosylation factor 3 (Acc# M74491) | 0.70 | 0.84 | 10/25 (40) | 1/10 (10) |
| Epidermal growth factor receptor substrate eps15 | 0.55 | 1.02 | 10/25 (40) | 1/10 (10) |
| (Acc# U07707) | | | | |
| Fibroblast muscle-type tropomyosin (Acc# | 0.62 | 0.79 | 10/25 (40) | 1/10 (10) |
| M12125) | | | | |
| mRNA for protein kinase C mu (Acc# X75756) | 0.64 | 0.81 | 10/25 (40) | 1/10 (10) |
| mRNA for RAB7 protein (Acc# X93499) | 0.63 | 0.94 | 10/25 (40) | 1/10 (10) |
| Wilm's tumor-related protein (QM) (Acc# | 0.78 | 0.99 | 10/25 (40) | 1/10 (10) |
| M64241) | | | | |
| Brain-expressed HHCPA78 homolog (Acc# | 0.38 | 0.92 | 15/25 (60) | 3/10 (30) |
| S73591) | | | | |
| TLS, translocated in liposarcoma (Acc# S62140) | 0.44 | 0.69 | 15/25 (60) | 3/10 (30) |
| Aspartyl beta-hydroxylase (Acc# U03109) | 0.73 | 1.23 | 7/25 (28) | 0/10 (0) |
| BACH1 (Acc# AB002803) | 0.84 | 1.05 | 7/25 (28) | 0/10 (0) |
| Bumetanide-sensitive Na-K-Cl cotransport (Acc# | 1.02 | 0.88 | 7/25 (28) | 0/10 (0) |
| U30246) | | | | |
| Desmin (Acc# AF006012) | 0.79 | 0.96 | 7/25 (28) | 0/10 (0) |
| Gamma SNAP (Acc# U78107) | 0.84 | 0.94 | 7/25 (28) | 0/10 (0) |
| Kruppel-associated box (KRAB), clone BRc1744 | 0.76 | 0.93 | 7/25 (28) | 0/10 (0) |
| (Acc# M67509) | | | | |
| Lung cancer antigen NY-LU-12 variant A (Acc# | 0.80 | 0.96 | 7/25 (28) | 0/10 (0) |
| AF042857) | | | | |
| mRNA for high mobility group protein | 0.75 | 0.72 | 7/25 (28) | 0/10 (0) |
| HMG2a(Acc# Y10043) | | | | |
| mRNA for KIAK0002 (Acc# D13639) | 0.66 | 0.91 | 7/25 (28) | 0/10 (0) |
| mRNA for XP-C repair complementing protein | 0.70 | 0.91 | 7/25 (28) | 0/10 (0) |
| (p58/HHR23B) (Acc# D21090) | | | | |
| Non-histone chromosomal protein (NHC) (Acc# | 0.77 | 1.13 | 7/25 (28) | 0/10 (0) |
| U90549) | | | | |
| PDGF associated protein (Acc# U41745) | 0.75 | 0.91 | 7/25 (28) | 0/10 (0) |
| Poly(ADP-ribose) polymerase (Acc# M18112) | 0.83 | 1.09 | 7/25 (28) | 0/10 (0) |
| Pulmonary surfactant-associated protein B (Acc# | 0.86 | 1.41 | 7/25 (28) | 0/10 (0) |
| J02761) | | | | |
| Steroid 5-alpha-reductase (Acc# M32313) | 0.72 | 0.98 | 7/25 (28) | 0/10 (0) |
| Syntenin (sycl) (Acc# AF000652) | 0.68 | 0.99 | 7/25 (28) | 0/10 (0) |
| TRAF family member-associated NF-kB activator | 0.88 | 0.96 | 7/25 (28) | 0/10 (0) |
| TANK (Acc# U63830) | | | | |
| cAMP phosphodiesterase (Pde7A2) (Acc# | 0.52 | 0.97 | 12/25 (48) | 0/10 (0) |
| U67932) | | | | |
| Cell matrix adhesion regulator variant (CMAR) | 0.57 | 0.74 | 12/25 (48) | 2/10 (20) |
| (Acc# AF034795) | | | | |
| HnRNP F protein (Acc# L28010) | 0.61 | 1.03 | 12/25 (48) | 2/10 (20) |
| Homolog of Drosophila enhancer of split m9/m10 | 0.67 | 0.70 | 12/25 (48) | 2/10 (20) |
| (Acc# U04241) | | | | |
| Melanoma antigen recognized by T-cells (MART- | 0.66 | 0.77 | 12/25 (48) | 2/10 (20) |
| 1) (Acc# U06452) | | | | |
| Nucleolar autoantigen No55 (Acc# U47621) | 0.70 | 0.79 | 12/25 (48) | 2/10 (20) |
| mRNA for HLA-DR antigens associated invariant | 0.44 | 0.79 | 14/25 (56) | 3/10 (30) |
| chain (p33) (Acc# X00497) | | | | |
| Synaptotagmin (Acc# M55047) | 0.40 | 0.73 | 14/25 (56) | 3/10 (30) |
| Acidic ribosomal phosphoprotein P2 (Acc# | 0.80 | 0.92 | 9/25 (36) | 1/10 (10) |
| M17887) | | | | |
| mRNA for KIAA0054 (Acc# D29677) | 0.89 | 0.95 | 9/25 (36) | 1/10 (10) |
| mRNA for NRAMP2 (Acc# AB004857) | 0.87 | 0.96 | 9/25 (36) | 1/10 (10) |
| mRNA for nucleolar protein hNop56 (Acc# | 0.72 | 0.85 | 9/25 (36) | 1/10 (10) |
| Y12065) | | | | |
| mRNA for tyrosine phosphatase(Acc# Z68092) | 0.61 | 0.72 | 9/25 (36) | 1/10 (10) |
| Myosin light chain 3 non-muscle (MC3 nm) (Acc# | 1.04 | 0.93 | 9/25 (36) | 1/10 (10) |
| M31212) | | | | |
| Placenta (Diff48) (Acc# U49187) | 0.66 | 0.98 | 9/25 (36) | 1/10 (10) |
| Protein phosphatase-1 catalytic subunit (Acc# | 0.70 | 0.97 | 9/25 (36) | 1/10 (10) |
| M63960) | | | | |
| Rearranged Ig heavy chain variable region (Acc# | 0.73 | 1.15 | 9/25 (36) | 1/10 (10) |
| Z14182) | | | | |
| Ser/Arg-related nuclear matrix protein (SRM160) | 0.61 | 0.72 | 9/25 (36) | 1/10 (10) |
| (Acc# AF048977) | | | | |
| Stratum corneum chymotryptic enzyme (Acc# | 0.68 | 1.04 | 9/25 (36) | 1/10 (10) |
| L33404) | | | | |
| Visinin-like peptide 1 homolog (Acc# U14747) | 0.78 | 1.01 | 9/25 (36) | 1/10 (10) |
| Zinc finger protein ZNF136 (Acc# U09367) | 0.72 | 1.01 | 9/25 (36) | 1/10 (10) |

B. Sequences Identified for Crohn's
Overexpressed Genes

| | | | | |
|---|---|---|---|---|
| GPx-4 mRNA (Acc# X71973) | 0.96 | 1.14 | 5/14 (36) | 0/10 (0) |
| mRNA for KIAA0380 (Acc# AB002378) | 1.13 | 1.06 | 5/14 (36) | 0/10 (0) |
| Cyclin-dependent protein kinase (Acc# J04973) | 1.35 | 1.70 | 5/14 (36) | 0/10 (0) |
| Pancreatic lipase related protein 1 (PLRP1) (Acc# | 1.19 | 0.97 | 5/14 (36) | 0/10 (0) |
| M93283) | | | | |
| Calnexin (Acc# L18887) | 1.32 | 1.09 | 5/14 (36) | 0/10 (0) |
| Cyclophilin-like protein (Acc# U39575) | 1.02 | 0.96 | 5/14 (36) | 0/10 (0) |
| Excitatory amino acid transporter 4 (Acc# | 1.38 | 1.05 | 5/14 (36) | 0/10 (0) |
| U18244) | | | | |
| Secretory carrier membrane protein (SCAMP3) | 1.17 | 0.82 | 5/14 (36) | 0/10 (0) |
| (Acc# AF005039) | | | | |
| Acidic calponin (Acc# S80562) | 1.61 | 0.92 | 6/14 (43) | 0/10 (0) |
| Vacuolar-type H(+) ATPase 115 kDa subunit | 1.28 | 0.77 | 6/14 (43) | 0/10 (0) |
| (Acc# U73006) | | | | |
| mRNA for KIAA0375 (Acc# AB002373) | 1.18 | 0.83 | 5/14 (36) | 0/10 (0) |
| P2x1 receptor (Acc# U45448) | 1.28 | 1.08 | 4/14 (29) | 0/10 (0) |
| Pilot mRNA (Acc# X63741) | 1.46 | 0.90 | 4/14 (29) | 0/10 (0) |
| Protein tyrosine kinase (Acc# M59371) | 1.24 | 0.92 | 4/14 (29) | 0/10 (0) |
| SOX9 (Acc# Z46629) | 1.14 | 0.90 | 4/14 (29) | 0/10 (0) |
| Tight junction (zonula occludens) protein ZO-1 | 1.04 | 1.09 | 4/14 (29) | 0/10 (0) |
| (Acc# L14837) | | | | |
| Arginosuccinate lyase (Acc# J03058) | 1.18 | 1.04 | 4/14 (29) | 0/10 (0) |
| Dioxin-inducible cytochrome P450 (CYP1B1) | 1.38 | 0.82 | 4/14 (29) | 0/10 (0) |
| (Acc# U39361) | | | | |
| Cu/Zn superoxide dismutase (SOD) (Acc# | 1.21 | 0.94 | 4/14 (29) | 0/10 (0) |
| X02317) | | | | |
| Fast MyBP-C (Acc# X73113) | 1.53 | 1.02 | 4/14 (29) | 0/10 (0) |
| IgG Fc binding protein (Acc# D84239) | 1.27 | 1.06 | 4/14 (29) | 0/10 (0) |
| KIAA0385 (Acc# AB002383) | 1.26 | 0.92 | 4/14 (29) | 0/10 (0) |
| KIAA0389 (Acc# AB002387) | 1.13 | 0.87 | 4/14 (29) | 0/10 (0) |
| Mr 110,000 (Acc# D64154) | 1.22 | 1.12 | 4/14 (29) | 0/10 (0) |
| Ubiquitin-conjugating enzyme UbcH7 (Acc# | 1.11 | 0.86 | 4/14 (29) | 0/10 (0) |
| AJ000519) | | | | |
| SCG10, neuron-specific growth-associated | 1.21 | 0.78 | 4/14 (29) | 0/10 (0) |
| protein/stathmin (Acc# S82024) | | | | |
| Homolog of Drosophila splicing regulator | 1.65 | 1.12 | 4/14 (29) | 0/10 (0) |
| suppressor-of-white-apricot (Acc# U08377) | | | | |
| PROS-27 (Acc# X59417). | 0.84 | 0.84 | 4/14 (29) | 0/10 (0) |
| (Acc# D21267) | 1.21 | 0.88 | 4/14 (29) | 0/10 (0) |
| Branched chain alpha-ketoacid dehydrogenase | 1.30 | 1.22 | 4/14 (29) | 0/10 (0) |
| kinase precursor (Acc# AF026548) | | | | |
| Cyclin H (Acc# U13737) | 1.34 | 1.41 | 4/14 (29) | 0/10 (0) |
| GPx-3 (Acc# X58295) | 1.12 | 1.06 | 4/14 (29) | 0/10 (0) |
| H4 (D10S170), putative cytoskeletal protein (Acc# | 1.26 | 0.88 | 4/14 (29) | 0/10 (0) |
| S72869) | | | | |
| Sm protein F (Acc# X85372) | 1.15 | 1.32 | 5/14 (36) | 1/10 (10) |
| GDNF family receptor alpha 2 (GFRalpha2) (Acc# | 1.45 | 1.04 | 5/14 (36) | 1/10 (10) |
| AF002700) | | | | |
| Brain cholecystokinin receptor (Acc# D13305) | 1.66 | 1.28 | 5/14 (36) | 1/10 (10) |
| Myelin basic protein (MBP) (Acc# M13577) | 1.01 | 1.03 | 5/14 (36) | 1/10 (10) |
| Rearranged metabotropic glutamate receptor type | 1.19 | 1.00 | 5/14 (36) | 1/10 (0) |
| II (Acc# L35318) | | | | |
| Alpha-2 collagen type VI-a’ gene, exon 1 (Acc# | 1.44 | 0.82 | 5/14 (36) | 1/10 (0) |
| M34573) | | | | |
| Complement component C3 mRNA, alpha and | 1.57 | 1.08 | 5/14 (36) | 1/10 (0) |
| beta (Acc# K02765) | | | | |
| Pyruvate dehydrogenase kinase isoenzyme 2 | 1.52 | 1.05 | 5/14 (36) | 1/10 (0) |
| (PDK2) (Acc# L42451) | | | | |

Underexpressed Genes

| | | | | |
|---|---|---|---|---|
| Epidermal growth factor receptor substrate (eps15) | 0.37 | 1.02 | 9/14 (64) | 1/10 (10) |
| (Acc# U07707) | | | | |
| Wilm's tumor-related protein (QM) (Acc# | 0.39 | 0.99 | 9/14 (64) | 1/10 (10) |
| M64241) | | | | |
| D53 (hD53) (Acc# M77830) | 0.46 | 1.49 | 7/14 (50) | 0/10 (0) |
| Desmin. (Acc# AF006012) | 0.48 | 0.96 | 7/14 (50) | 0/10 (0) |
| hCDC10, CDC10 homolog. (Acc# S72008) | 0.57 | 0.73 | 7/14 (50) | 0/10 (0) |
| mRNA for C3G protein. (Acc# D21239) | 0.47 | 1.01 | 7/14 (50) | 0/10 (0) |
| mRNA for mosaic protein LR11. (Acc# Y08110) | 0.62 | 1.45 | 7/14 (50) | 0/10 (0) |
| Sorting nexin 2 (SNX2) (Acc# AF043453) | 0.61 | 1.12 | 7/14 (50) | 0/10 (0) |
| Methionine aminopeptidase. (Acc# U29607) | 0.34 | 0.90 | 8/14 (57) | 1/10 (10) |
| Homolog of Drosophila enhancer of split m9/m10. | 0.41 | 0.70 | 9/14 (64) | 2/10 (20) |
| (Acc# U04241) | | | | |
| Carbonic anhydrase II. (Acc# J03037) | 0.60 | 1.28 | 6/14 (43) | 0/10 (0) |
| mRNA for HBp15/L22. (Acc# D17652) | 0.92 | 1.33 | 6/14 (43) | 0/10 (0) |
| mRNA for maleylacetoacetate isomerase. (Acc# | 0.65 | 1.13 | 6/14 (43) | 0/10 (0) |
| AJ001888) | | | | |
| mRNA for SHPS-1. (Acc# D86043) | 0.67 | 1.49 | 6/14 (43) | 0/10 (0) |
| Sodium-hydrogen exchanger 6 (NHE-6) mRNA, | 0.91 | 0.84 | 6/14 (43) | 0/10 (0) |
| nuclear gene encoding mitochondrial protein. | | | | |
| (Acc# AF030409) | | | | |
| Sorbitol dehydrogenase gene. (Acc# U07361) | 0.66 | 1.38 | 6/14 (43) | 0/10 (0) |
| Arp2/3 protein complex subunit p34-Arc (ARC34) | 0.76 | 1.49 | 7/14 (50) | 1/10 (10) |
| (Acc# AF006085) | | | | |
| mRNA for nucleolar protein hNop56. (Acc# | 0.61 | 0.85 | 7/14 (50) | 1/10 (10) |
| Y12065) | | | | |
| mRNA for RAB7 protein. (Acc# X93499) | 0.40 | 0.94 | 7/14 (50) | 1/10 (10) |
| Placenta (Diff48) (Acc# U49187) | 0.48 | 0.98 | 7/14 (50) | 1/10 (10) |
| Visinin-like peptide 1 homolog. (Acc# U14747) | 0.61 | 1.01 | 7/14 (50) | 1/10 (10) |
| HnRNP F protein. (Acc# L28010) | 0.44 | 1.03 | 8/14 (50) | 2/10 (20) |
| SREBP-1 (Acc# U00968) | 0.43 | 1.30 | 8/14 (50) | 2/10 (20) |
| Aldolase A (Acc# M11560) | 0.92 | 1.64 | 5/14 (36) | 0/10 (0) |
| BACH1. (Acc# AB002803) | 0.69 | 1.05 | 5/14 (36) | 0/10 (0) |
| Dynamin (DNM) (Acc# S72422) | 0.83 | 1.04 | 5/14 (36) | 0/10 (0) |
| G protein gamma-4 subunit. (Acc# U31382) | 0.73 | 0.99 | 5/14 (36) | 0/10 (0) |
| Kruppel-associated box (KRAB), clone BRc1744. | 0.71 | 0.93 | 5/14 (36) | 0/10 (0) |
| (Acc# M67509) | | | | |
| Lung cancer antigen NY-LU-12 variant A. (Acc# | 0.67 | 0.96 | 5/14 (36) | 0/10 (0) |
| AF042857) | | | | |
| mRNA for KIAK0002 gene. (Acc# D13639) | 0.66 | 0.91 | 5/14 (36) | 0/10 (0) |
| mRNA for MLL. (Acc# D14540) | 0.75 | 1.31 | 5/14 (36) | 0/10 (0) |
| mRNA for Na, K-ATPase alpha-subunit. (Acc# | 0.88 | 1.38 | 5/14 (36) | 0/10 (0) |
| X04297) | | | | |
| mRNA for novel DNA binding protein. (Acc# | 0.73 | 1.50 | 5/14 (36) | 0/10 (0) |
| X63071) | | | | |
| mRNA for ORF, Xq terminal portion. (Acc# | 0.85 | 1.27 | 5/14 (36) | 0/10 (0) |
| D16469) | | | | |
| Non-histone chromosomal protein (NHC) (Acc# | 0.78 | 1.13 | 5/14 (36) | 0/10 (0) |
| U90549) | | | | |
| Nuclear receptor hTAK1 (hTAK1) (Acc# | 0.91 | 1.53 | 5/14 (36) | 0/10 (0) |
| U10990) | | | | |
| PKC alpha mRNA for protein kinase C alpha. | 0.58 | 0.88 | 5/14 (36) | 0/10 (0) |
| (Acc# X52479) | | | | |
| poly(ADP-ribose) polymerase. (Acc# M18112) | 0.68 | 1.09 | 5/14 (36) | 0/10 (0) |
| Rac3 (RAC3) (Acc# AF008591) | 0.92 | 0.99 | 5/14 (36) | 0/10 (0) |
| Rearranged mRNA for glutamine synthase. (Acc# | 0.93 | 1.32 | 5/14 (36) | 0/10 (0) |
| X59834) | | | | |
| Sarcosin. (Acc# AF056929) | 0.85 | 1.29 | 5/14 (36) | 0/10 (0) |
| Sec61-complex beta-subunit. (Acc# L25085) | 0.79 | 1.10 | 5/14 (36) | 0/10 (0) |
| Syntenin (sycl) (Acc# AF00652) | 0.62 | 0.99 | 5/14 (36) | 0/10 (0) |
| Translation repressor NAT1. (Acc# U76111) | 0.87 | 1.03 | 5/14 (36) | 0/10 (0) |
| mRNA for 23 kD highly basic protein. (Acc# | 0.43 | 0.84 | 9/14 (64) | 3/10 (30) |
| X56932) | | | | |
| 26 S protease subunit 5b, 50 kda subunit. (Acc# | 0.57 | 0.88 | 6/14 (43) | 1/10 (10) |
| S79862) | | | | |
| Acidic ribosomal phosphoprotein P2. (Acc# | 0.85 | 1.08 | 6/14 (43) | 1/10 (10) |
| M17887) | | | | |
| Chaperonin protein (Tcp20) gene complete cds. | 0.73 | 1.04 | 6/14 (43) | 1/10 (10) |
| (Acc# L27006) | | | | |
| Cytochrome c oxidase subunit Vb (coxVb) (Acc# | 0.66 | 1.09 | 6/14 (43) | 1/10 (10) |
| M19961) | | | | |
| DNJ3/CPR3. (Acc# AF011793) | 0.83 | 0.97 | 6/14 (43) | 1/10 (10) |
| Endothelial cell protein C/APC receptor (EPCR) | 0.58 | 1.16 | 6/14 (43) | 1/10 (10) |
| (Acc# L35545) | | | | |
| Fibroblast muscle-type tropomyosin. (Acc# | 0.63 | 0.79 | 6/14 (43) | 1/10 (10) |
| M12125) | | | | |
| Hbrm mRNA, (Acc# X72889) | 0.58 | 0.95 | 6/14 (43) | 1/10 (10) |
| mRNA encoding GPI-anchored protein p137. | 0.66 | 0.69 | 6/14 (43) | 1/10 (10) |
| (Acc# Z48042) | | | | |
| mRNA for Clock. (Acc# AB05535) | 0.72 | 1.21 | 6/14 (43) | 1/10 (10) |
| mRNA for ribosomal protein L7. (Acc# X52967) | 0.65 | 1.20 | 6/14 (43) | 1/10 (10) |
| mRNA for skeletal beta-tropomyosin. (Acc# | 0.54 | 0.76 | 6/14 (43) | 1/10 (10) |
| X06825) | | | | |
| Protein phosphatase-1 catalytic subunit. (Acc# | 0.57 | 0.97 | 6/14 (43) | 1/10 (10) |
| M63960) | | | | |
| Rearranged Ig heavy chain variable region (VDJ) | 0.67 | 1.15 | 6/14 (43) | 1/10 (10) |
| (Acc# Z14182) | | | | |
| Translation initiation factor 5 (eIF5) (Acc# | 0.75 | 0.81 | 6/14 (43) | 1/10 (10) |
| U49436) | | | | |
| Adult skeletal muscle alpha-actin mRNA. (Acc# | 0.15 | 0.95 | 10/14 (71) | 4/10 (40) |
| J00068) | | | | |
| mRNA for vascular smooth muscle alpha-actin. | 0.17 | 0.86 | 10/14 (71) | 4/10 (40) |
| (Acc# X13839) | | | | |
| 90-kDa heat-shock protein gene, cDNA. (Acc# | 0.55 | 0.82 | 7/14 (50) | 2/10 (20) |
| M16660) | | | | |
| Adhalin mRNA with an R266C mutation causing a | 0.60 | 0.74 | 7/14 (50) | 2/10 (20) |
| mild form of autosomal recessive limb-girdle | | | | |
| muscular dystrophy (Acc# L46810) | | | | |
| Alkali myosin light chain 1. (Acc# M20642) | 0.50 | 0.72 | 7/14 (50) | 2/10 (20) |
| MHC protein homologous to chicken B complex | 0.48 | 1.59 | 7/14 (50) | 2/10 (20) |
| protein. (Acc# M24194) | | | | |
| mRNA for insulin-like growth factor II receptor. | 0.50 | 0.87 | 7/14 (50) | 2/10 (20) |
| (Acc# Y00285) | | | | |
| Phospholipase A2. (Acc# M86400) | 0.56 | 1.17 | 7/14 (50) | 2/10 (20) |
| SIL. (Acc# M74558) | 0.56 | 0.81 | 7/14 (50) | 2/10 (20) |
| 26-kDa cell surface protein TAPA-1. (Acc# | 1.09 | 1.01 | 4/14 (29) | 0/10 (0) |
| M33680) | | | | |
| Achaete scute homologous protein (ASH1) (Acc# | 0.82 | 0.70 | 4/14 (29) | 0/10 (0) |
| L08424) | | | | |
| ADP-ribosylation factor 1 (ARF1) (Acc# | 0.90 | 1.04 | 4/14 (29) | 0/10 (0) |
| M36340) | | | | |
| AMP deaminase isoform L, alternatively spliced | 0.92 | 0.84 | 4/14 (29) | 0/10 (0) |
| (AMPD2) mRNA, exons 1B, 2 and 3 (Acc# | | | | |
| U16268) | | | | |
| Arginine-rich nuclear protein. (Acc# M74002) | 0.92 | 1.02 | 4/14 (29) | 0/10 (0) |
| Beta-myosin heavy chain (MYH7) (Acc# | 0.78 | 0.84 | 4/14 (29) | 0/10 (0) |
| M58018) | | | | |
| Dihydropteridine reductase (hDHPR) (Acc# | 0.87 | 0.82 | 4/14 (29) | 0/10 (0) |
| M16447) | | | | |
| Dynamitin. (Acc# U50733) | 0.81 | 0.95 | 4/14 (29) | 0/10 (0) |
| Erythroblastosis virus oncogene homolog 2 (ets-2) | 0.87 | 1.03 | 4/14 (29) | 0/10 (0) |
| (Acc# J04102) | | | | |
| Fibroblast collagenase inhibitor. (Acc# M12670) | 0.93 | 1.12 | 4/14 (29) | 0/10 (0) |
| FUSE binding protein. (Acc# U05040) | 0.90 | 1.00 | 4/14 (29) | 0/10 (0) |
| fvt1 mRNA. (Acc# X63657) | 0.69 | 0.90 | 4/14 (29) | 0/10 (0) |
| G/T mismatch-specific thymine DNA glycosylase. | 0.77 | 0.85 | 4/14 (29) | 0/10 (0) |
| (Acc# U51166) | | | | |
| GM-CSF receptor. (Acc# M64445) | 0.88 | 1.14 | 4/14 (29) | 0/10 (0) |
| GTF3A mRNA for Xenopus transcription factor | 0.78 | 1.24 | 4/14 (29) | 0/10 (0) |
| IIIA homologue. (Acc# D32257) | | | | |
| Heme oxygenase-2. (Acc# S34389) | 0.76 | 0.99 | 4/14 (29) | 0/10 (0) |
| inosine-5′-monophosphate dehydrogenase (IMP) | 0.63 | 1.00 | 4/14 (29) | 0/10 (0) |
| (Acc# J04208) | | | | |
| Jumonji putative protein (jumonji) (Acc# | 1.05. | 1.04 | 4/14 (29) | 0/10 (0) |
| U57592) | | | | |
| Lymph node homing receptor. (Acc# M25280) | 0.69 | 2.09 | 4/14 (29) | 0/10 (0) |
| Lymphoid nuclear protein (LAF-4) (Acc# | 1.00 | 1.40 | 4/14 (29) | 0/10 (0) |
| U34360) | | | | |
| Mitochondrial matrix protein P1 (nuclear encoded) | 0.76 | 1.00 | 4/14 (29) | 0/10 (0) |
| (Acc# M22382) | | | | |
| mRNA for 26S proteasome subunit p40.5. (Acc# | 0.82 | 0.91 | 4/14 (29) | 0/10 (0) |
| AB009398) | | | | |
| mRNA for axonal transporter of synaptic vesicles. | 0.76 | 1.19 | 4/14 (29) | 0/10 (0) |
| (Acc# X70840) | | | | |
| mRNA for DEC1. (Acc# AB004066) | 0.86 | 1.33 | 4/14 (29) | 0/10 (0) |
| mRNA for GARS-AIRS-GART. (Acc# X54199) | 1.13 | 1.00 | 4/14 (29) | 0/10 (0) |
| mRNA for glutathione peroxidase. (Acc# D00632) | 1.05 | 1.26 | 4/14 (29) | 0/10 (0) |
| mRNA for integrin beta(4)subunit. (Acc# X51841) | 1.27 | 1.03 | 4/14 (29) | 0/10 (0) |
| mRNA for keratin-related protein. (Acc# X62571) | 0.92 | 1.04 | 4/14 (29) | 0/10 (0) |
| mRNA for KIAA0210 gene. (Acc# D86965) | 1.09 | 0.92 | 4/14 (29) | 0/10 (0) |
| mRNA for lipoprotein lipase (EC 3.1.1.34) (Acc# | 0.98 | 0.97 | 4/14 (29) | 0/10 (0) |
| X54516) | | | | |
| mRNA for protein kinase CK1. (Acc# X80693) | 0.76 | 0.79 | 4/14 (29) | 0/10 (0) |
| mRNA for protein phosphatase 2A (beta-type) | 0.74 | 1.07 | 4/14 (29) | 0/10 (0) |
| (Acc# X12656) | | | | |
| mRNA for succinyl CoA synthetase. (Acc# | 1.40 | 1.56 | 4/14 (29) | 0/10 (0) |
| Z68204) | | | | |
| mRNA for transducin (beta) like 1 protein. (Acc# | 1.00 | 0.88 | 4/14 (29) | 0/10 (0) |
| Y12781) | | | | |
| mRNA for vacuolar H+ ATPase E subunit. (Acc# | 0.95 | 1.15 | 4/14 (29) | 0/10 (0) |
| X76228) | | | | |
| Nuclear-encoded mitochondrial NADH- | 1.13 | 1.37 | 4/14 (29) | 0/10 (0) |
| ubiquinone reductase 24 Kd subunit. (Acc# | | | | |
| M22538) | | | | |
| p70 (ST5) mRNA, alternatively spliced. (Acc# | 0.78 | 0.93 | 4/14 (29) | 0/10 (0) |
| U15779) | | | | |
| Partial C1 mRNA. (Acc# X78817) | 0.85 | 0.99 | 4/14 (29) | 0/10 (0) |
| PBX3 mRNA. (Acc# X59841) | 0.96 | 1.02 | 4/14 (29) | 0/10 (0) |
| PDGF associated protein. (Acc# U41745) | 0.71 | 0.91 | 4/14 (29) | 0/10 (0) |
| Phosphatidylethanolamine binding protein mRNA. | 0.81 | 0.77 | 4/14 (29) | 0/10 (0) |
| (Acc# X75252) | | | | |
| Phospholipase c delta 1. (Acc# U09117) | 0.77 | 1.05 | 4/14 (29) | 0/10 (0) |
| Pulmonary surfactant-associated protein B (SP-B) | 0.89 | 1.41 | 4/14 (29) | 0/10 (0) |
| (Acc# J02761) | | | | |
| PYRIN (MEFV) (Acc# AF018080) | 0.69 | 0.80 | 4/14 (29) | 0/10 (0) |
| RGP3. (Acc# U27655) | 0.97 | 1.03 | 4/14 (29) | 0/10 (0) |
| RP3. (Acc# U02556) | 0.83 | 0.99 | 4/14 (29) | 0/10 (0) |
| sin3 associated polypeptide p18 (SAP18) (Acc# | 1.18 | 0.95 | 4/14 (29) | 0/10 (0) |
| U96915) | | | | |
| Steroid 5-alpha-reductase. (Acc# M32313) | 0.80 | 0.98 | 4/14 (29) | 0/10 (0) |
| TFIID subunit TAFII55 (TAFII55) (Acc# | 0.80 | 0.93 | 4/14 (29) | 0/10 (0) |
| U18062) | | | | |
| TRAF family member-associated NF-kB activator | 0.90 | 0.96 | 4/14 (29) | 0/10 (0) |
| TANK (Acc# U63830) | | | | |
| TRAIL receptor 2. (Acc# AF016266) | 0.76 | 0.82 | 4/14 (29) | 0/10 (0) |
| Brain-expressed HHCPA78 homolog. (Acc# | 0.38 | 0.92 | 8/14 (57) | 3/10 (30) |
| S73591) | | | | |
| Inducible protein. (Acc# L47738) | 0.48 | 0.78 | 8/14 (57) | 3/10 (30) |
| mRNA for HLA-DR antigens associated invariant | 0.29 | 0.79 | 8/14 (57) | 3/10 (30) |
| chain (p33) (Acc# X00497) | | | | |
| mRNA for ribosomal protein L14. (Acc# D87735) | 0.41 | 0.86 | 8/14 (57) | 3/10 (30) |
| mRNA for ribosomal protein L3. (Acc# X73460) | 0.38 | 0.80 | 8/14 (57) | 3/10 (30) |
| Myosin alkali light chain (ventricular) (Acc# | 0.41 | 0.95 | 8/14 (57) | 3/10 (30) |
| M24122) | | | | |
| TLS, translocated in liposarcoma. (Acc# | 0.45 | 0.69 | 8/14 (57) | 3/10 (30) |
| AF047448) | | | | |
| (p23) (Acc# L24804) | 1.11 | 1.06 | 5/14 (36) | 1/10 (10) |
| Acetolactate synthase homolog. (Acc# U61623) | 0.99 | 1.17 | 5/14 (36) | 1/10 (10) |
| ADP-ribosylation factor 3. (Acc# M74491) | 0.65 | 0.84 | 5/14 (36) | 1/10 (10) |
| Alternatively spliced trp-1 protein and unspliced | 0.72 | 1.08 | 5/14 (36) | 1/10 (10) |
| trp-1 protein (trp-1) (Acc# U31110) | | | | |
| Butyrophilin (BTF4) (Acc# U90546) | 0.58 | 0.74 | 5/14 (36) | 1/10 (10) |
| CL 100 mRNA for protein tyrosine phosphatase. | 0.77 | 1.18 | 5/14 (36) | 1/10 (10) |
| (Acc# X68277) | | | | |
| Creatine transporter. (Acc# S71018) | 0.66 | 1.00 | 5/14 (36) | 1/10 (10) |
| Desmosome associated protein pinin. (Acc# | 0.84 | 1.07 | 5/14 (36) | 1/10 (10) |
| U46461) | | | | |
| Deubiquitinating enzyme UnpEL (UNP) (Acc# | 0.86 | 1.12 | 5/14 (36) | 1/10 (10) |
| U37707) | | | | |
| ERK1 mRNA for protein serine/threonine kinase. | 0.80 | 1.03 | 5/14 (36) | 1/10 (10) |
| (Acc# X60188) | | | | |
| ERK3 protein kinase. (Acc# L77964) | 0.67 | 1.07 | 5/14 (36) | 1/10 (10) |
| EWS mRNA. (Acc# X66899) | 0.90 | 0.77 | 5/14 (36) | 1/10 (10) |
| hnRNP core protein A1. (Acc# X06747) | 0.57 | 0.82 | 5/14 (36) | 1/10 (10) |
| L23 mRNA for putative ribosomal protein. (Acc# | 0.86 | 1.00 | 5/14 (36) | 1/10 (10) |
| X53777) | | | | |
| mRNA encoding IMP: pyrophosphate | 1.02 | 0.91 | 5/14 (36) | 1/10 (10) |
| phosphoribosyltransferase E.C. 2.4.2.8. (Acc# | | | | |
| V00530) | | | | |
| mRNA for lactate dehydrogenase B (LDH-B) | 0.88 | 1.02 | 5/14 (36) | 1/10 (10) |
| (Acc# Y00711) | | | | |
| mRNA for LAK-1. (Acc# AB005754) | 0.66 | 0.97 | 5/14 (36) | 1/10 (10) |
| mRNA for laminin M chain (merosin) (Acc# | 0.90 | 0.92 | 5/14 (36) | 1/10 (10) |
| Z26653) | | | | |
| mRNA for neuropeptide Y-like receptor. (Acc# | 0.83 | 1.34 | 5/14 (36) | 1/10 (10) |
| X71635) | | | | |
| mRNA for SMT3B protein. (Acc# X99585) | 1.09 | 0.75 | 5/14 (36) | 1/10 (10) |
| mRNA for tyrosine phosphatase. (Acc# Z68092) | 0.61 | 0.72 | 5/14 (36) | 1/10 (10) |
| Neural cell adhesion molecule (CALL) (Acc# | 1.03 | 1.06 | 5/14 (36) | 1/10 (10) |
| AF002246) | | | | |
| NMB mRNA. (Acc# X76534) | 1.07 | 0.76 | 5/14 (36) | 1/10 (10) |
| Of PLZF gene encoding kruppel-like zinc finger | 0.76 | 0.86 | 5/14 (36) | 1/10 (10) |
| protein. (Acc# Z19002) | | | | |
| PDCD2, programmed cell death-2/Rp8 homolog. | 0.63 | 0.76 | 5/14 (36) | 1/10 (10) |
| (Acc# S78085) | | | | |
| Protein kinase C-L (PRKCL) (Acc# M55284) | 0.86 | 0.89 | 5/14 (36) | 1/10 (10) |
| Ribosomal protein L12. (Acc# L06505) | 1.03 | 1.23 | 5/14 (36) | 1/10 (10) |
| Ribosomal protein L23a. (Acc# U37230) | 0.88 | 1.32 | 5/14 (36) | 1/10 (10) |
| Ribosomal protein L30. (Acc# M94314) | 1.05 | 1.30 | 5/14 (36) | 1/10 (10) |
| Ser/Arg-related nuclear matrix protein (SRM160) | 0.66 | 0.72 | 5/14 (36) | 1/10 (10) |
| (Acc# AF048977) | | | | |
| Succinate dehydrogenase flavoprotein subunit | 0.80 | 1.11 | 5/14 (36) | 1/10 (10) |
| (SDH) (Acc# L21936) | | | | |
| Transposon L1.1 with a base deletion relative to | 0.82 | 1.09 | 5/14 (36) | 1/10 (10) |
| L1.2B resulting in a premature stop codon in the | | | | |
| coding region. (Acc# M80340) | | | | |

C. Sequences Identified for Ulcerative Colitis
Overexpressed Genes

| | | | | |
|---|---|---|---|---|
| Beta-sarcoglycan A3b. (Acc# U31116) | 2.21 | | 7/11 (64) | 0/10 (0) |
| (p23) (Acc# L24804) | 2.34 | 1.06 | 8/11 (73) | 1/10 (10) |
| Glial fibrillary acidic protein (GFAP) (Acc# | 2.41 | 0.93 | 6/11 (55) | 0/10 (0) |
| J04569) | | | | |
| mRNA for serine/threonine protein kinase. | 2.15 | 1.20 | 6/11 (55) | 0/10 (0) |
| (Acc# D86550) | | | | |
| mRNA for ZIP-kinase. (Acc# AB007144) | 2.16 | 0.66 | 6/11 (55) | 0/10 (0) |
| Tumor susceptiblity protein (TSG101) (Acc# | 2.16 | 1.27 | 6/11 (55) | 0/10 (0) |
| U82130) | | | | |
| 14-3-3 protein epsilon isoform. (Acc# | 2.74 | 1.08 | 7/11 (64) | 1/10 (10) |
| U43399) | | | | |
| Acidic calponin. (Acc# S80562) | 2.17 | 0.92 | 7/11 (64) | 1/10 (10) |
| mRNA for cytosolic asparaginyl-tRNA | 2.02 | 0.95 | 7/11 (64) | 1/10 (10) |
| synthetase. (Acc# AJ000334) | | | | |
| rab2 mRNA, YPT1-related and member of ras | 2.18 | 1.16 | 7/11 (64) | 1/10 (10) |
| family. (Acc# X12953) | | | | |
| Replication protein A 32-kDa subunit. (Acc# | 2.13 | 1.28 | 7/11 (64) | 1/10 (10) |
| J05249) | | | | |
| Sodium/potassium-transporting ATPase beta-3 | 2.61 | 1.00 | 7/11 (64) | 1/10 (10) |
| subunit. (Acc# U51478) | | | | |
| Testicular angiotensin converting enzyme | 2.61 | 1.16 | 7/11 (64) | 1/10 (10) |
| mRNA (5′ variant) (Acc# M26658) | | | | |
| mRNA for lymphocte activation marker Blast- | 2.42 | 1.34 | 8/11 (73) | 2/10 (20) |
| 1. (Acc# X06341) | | | | |
| rab11a GTPase. (Acc# AF000231) | 2.72 | 1.15 | 8/11 (73) | 2/10 (20) |
| mRNA for APRIL protein. (Acc# Y07969) | 1.82 | 1.26 | 5/11 (45) | 0/10 (0) |
| mRNA for KIAA0091 gene. (Acc# D42053) | 1.47 | 1.37 | 5/11 (45) | 0/10 (0) |
| mRNA for KIAA0168 gene. (Acc# D79990) | 1.97 | 0.81 | 5/11 (45) | 0/10 (0) |
| mRNA for skeletal muscle 190 kD protein. | 1.59 | 1.11 | 5/11 (45) | 0/10 (0) |
| (Acc# X69090) | | | | |
| mRNA for subunit C of vacuolar proton- | 1.74 | 1.28 | 5/11 (45) | 0/10 (0) |
| ATPase V1 domain. (Acc# X69151) | | | | |
| Proto-oncogene Bcd orf1 and orf2. (Acc# | 1.78 | 0.68 | 5/11 (45) | 0/10 (0) |
| U51869) | | | | |
| Zinc finger protein 216 splice variant 2 | 1.03 | 1.06 | 5/11 (45) | 0/10 (0) |
| (ZNF216) (Acc# AF06234) | | | | |
| Lipoxygenase. (Acc# J03600) | 1.79 | 0.95 | 5/11 (45) | 0/10 (0) |
| GPx-4 mRNA for phospholipid hydroperoxide | 0.82 | 1.14 | 5/11 (45) | 0/10 (0) |
| glutathione peroxidase. (Acc# X71973) | | | | |
| Autosomal dominant polycystic kidney | 1.75 | 1.39 | 5/11 (45) | 0/10 (0) |
| disease protein 1 (PKD1) (Acc# U24497) | | | | |
| Anion exchanger 3 brain isoform (bAE3) | 1.68 | 1.06 | 5/11 (45) | 0/10 (0) |
| (Acc# U05596) | | | | |
| mRNA for apoptosis specific protein. (Acc# | 2.50 | 1.21 | 6/11 (54) | 01/10 (10) |
| Y11588) | | | | |
| mRNA for G13 protein. (Acc# X98054) | 2.34 | 1.38 | 6/11 (54) | 01/10 (10) |
| mRNA for golgi alpha-mannosidaseII. (Acc# | 2.17 | 0.95 | 6/11 (54) | 01/10 (10) |
| D63998) | | | | |
| Myosin alkali light chain (ventricular) (Acc# | 2.05 | 0.95 | 6/11 (54) | 01/10 (10) |
| M24122) | | | | |
| Rapamycin binding protein (FK506) (Acc# | 2.26 | 1.25 | 6/11 (54) | 1/10 (10) |
| M96256) | | | | |
| Ribosomal protein L23-related. (Acc# | 2.21 | 0.90 | 6/11 (54) | 1/10 (10) |
| U26596) | | | | |
| RSU-1/RSP-1. (Acc# L12535) | 2.39 | 1.11 | 6/11 (54) | 1/10 (10) |
| (clone S171) (Acc# L40393) | 2.30 | 1.23 | 7/11 (64) | 2/10 (20) |
| BCL2/adenovirus E1B 19 kD-interacting | 2.88 | 1.18 | 7/11 (64) | 2/10 (20) |
| protein 2 (BNIP2) (Acc# U15173) | | | | |
| Deubiquitinating enzyme UnpEL (UNP) | 2.49 | 1.12 | 7/11 (64) | 2/10 (20) |
| (Acc# U37707) | | | | |
| mRNA encoding IMP: pyrophosphate | 2.60 | 0.91 | 7/11 (64) | 2/10 (20) |
| phosphoribosyltransferase E.C. 2.4.2.8. (Acc# | | | | |
| V00530) | | | | |
| mRNA for insulinoma pre-proinsulin. (Acc# | 3.37 | 1.32 | 7/11 (64) | 2/10 (20) |
| X70508) | | | | |
| N-formylpeptide receptor (fMLP-R26) (Acc# | 2.99 | 1.62 | 7/11 (64) | 2/10 (20) |
| M60627) | | | | |
| Protein-tyrosine kinase (JAK1) (Acc# | 2.47 | 1.18 | 7/11 (64) | 2/10 (20) |
| M64174) | | | | |
| Ribosomal protein L18 (RPL18) (Acc# | 2.79 | 1.42 | 7/11 (64) | 2/10 (20) |
| L11566) | | | | |
| U2 small nuclear RNA-associated B” antigen. | 2.93 | 1.17 | 7/11 (64) | 2/10 (20) |
| (Acc# M15841) | | | | |
| glutathione-S-transferase homolog. (Acc# | 2.46 | 1.48 | 8/11 (73) | 3/10 (30) |
| U90313) | | | | |
| AUH mRNA. (Acc# X79888) | 1.46 | 1.28 | 4/11 (36) | 0/10 (0) |
| cadherin-4. (Acc# L34059) | 1.58 | 0.97 | 4/11 (36) | 0/10 (0) |
| Clone 22 mRNA, alternative splice variant | 1.14 | 0.92 | 4/11 (36) | 0/10 (0) |
| beta-2. (Acc# AF009427) | | | | |
| dlg3. (Acc# J03068) | 1.70 | 1.11 | 4/11 (36) | 0/10 (0) |
| FUSE binding protein. (Acc# U05040) | 1.66 | 1.00 | 4/11 (36) | 0/10 (0) |
| Inhibitor of apoptosis protein 2. (Acc# | 1.67 | 1.15 | 4/11 (36) | 0/10 (0) |
| U45879) | | | | |
| KCNC1, voltage-gated potassium channel. | 1.19 | 1.41 | 4/11 (36) | 0/10 (0) |
| (Acc# S56770) | | | | |
| Kidney mRNA for catalase. (Acc# X04076) | 1.28 | 1.00 | 4/11 (36) | 0/10 (0) |
| Kruppel-related zinc finger protein hcKrox. | 0.98 | 0.93 | 4/11 (36) | 0/10 (0) |
| (Acc# AF007833) | | | | |
| mRNA for APO-1 cell surface antigen. (Acc# | 0.89 | 1.03 | 4/11 (36) | 0/10 (0) |
| X63717) | | | | |
| mRNA for ceramide glucosyltransferase. | 1.14 | 0.72 | 4/11 (36) | 0/10 (0) |
| (Acc# D50840) | | | | |
| mRNA for lipoprotein lipase (EC 3.1.1.34) | 1.00 | 0.97 | 4/11 (36) | 0/10 (0) |
| (Acc# X14390) | | | | |
| mRNA for phosphoriobosyl pyrophosphate | 1.70 | 0.98 | 4/11 (36) | 0/10 (0) |
| synthetase subunit II (EC 2.7.6.1) (Acc# | | | | |
| Y00971) | | | | |
| mRNA for platelet-type phosphofructokinase. | 1.56 | 1.57 | 4/11 (36) | 0/10 (0) |
| (Acc# D25328) | | | | |
| mRNA for protein kinase C zeta. (Acc# | 1.35 | 1.06 | 4/11 (36) | 0/10 (0) |
| Z15108) | | | | |
| mRNA for small GTP-binding protein, S10. | 1.66 | 1.37 | 4/11 (36) | 0/10 (0) |
| (Acc# D14889) | | | | |
| mRNA for striatin. (Acc# AJ223814) | 1.49 | 1.22 | 4/11 (36) | 0/10 (0) |
| mss4, Zn2 + binding protein/guanine | 1.07 | 0.97 | 4/11 (36) | 0/10 (0) |
| nucleotide exchange factor. (Acc# S78873) | | | | |
| Nucleosome assembly protein 2. (Acc# | 1.45 | 0.96 | 4/11 (36) | 0/10 (0) |
| U77456) | | | | |
| Phospholipase C-gamma. (Acc# M34667) | 1.17 | 0.99 | 4/11 (36) | 0/10 (0) |
| Preprocarboxypeptidase A2 (proCPA2) (Acc# | 1.61 | 1.36 | 4/11 (36) | 0/10 (0) |
| U19977) | | | | |
| Protein phosphatase 2C alpha. (Acc# S87759) | 1.79 | 0.89 | 4/11 (36) | 0/10 (0) |
| Sec62 (Sec62) (Acc# U93239) | 1.62 | 1.16 | 4/11 (36) | 0/10 (0) |
| Succinyl CoA: 3-oxoacid CoA transferase | 1.06 | 0.95 | 4/11 (36) | 0/10 (0) |
| precursor (OXCT) (Acc# U62961) | | | | |
| Secretory carrier-associated membrane protein | 1.42 | 1.07 | 4/11 (36) | 0/10 (0) |
| (SCAMP) (Acc# AF038966) | | | | |
| Succinyl CoA: 3-oxoacid CoA transferase | 1.06 | 0.95 | 4/11 (36) | 0/10 (0) |
| precursor (OXCT) (Acc# U62961) | | | | |
| X104. (Acc# L27476) | 1.72 | 0.88 | 4/11 (36) | 0/10 (0) |
| (clone pA3) protein disulfide isomerase | 1.50 | 1.02 | 5/11 (45) | 1/10 (10) |
| related protein (ERp72) (Acc# J05016) | | | | |
| Adenylate kinase 2 (adk2) (Acc# U39945) | 1.20 | 1.10 | 5/11 (45) | 1/10 (10) |
| Cell cycle progression 2 protein (CPR2) | 1.68 | 1.00 | 5/11 (45) | 1/10 (10) |
| (Acc# AF011792) | | | | |
| Clone 23733. (Acc# U79274) | 1.63 | 1.29 | 5/11 (45) | 1/10 (10) |
| eps8 binding protein e3B1. (Acc# AF006516) | 1.92 | 1.42 | 5/11 (45) | 1/10 (10) |
| GTP-binding protein (RALB) (Acc# | 1.21 | 0.91 | 5/11 (45) | 1/10 (10) |
| M35416) | | | | |
| Histone 2A-like protein (H2A/1) (Acc# | 1.90 | 1.21 | 5/11 (45) | 1/10 (10) |
| U90551) | | | | |
| Leukemia virus receptor 2 (GLVR2) (Acc# | 1.65 | 1.05 | 5/11 (45) | 1/10 (10) |
| L20852) | | | | |
| mRNA for carboxyl methyltransferase. (Acc# | 1.95 | 1.17 | 5/11 (45) | 1/10 (10) |
| D13892) | | | | |
| mRNA for EHK-1 receptor tyrosine kinase. | 1.95 | 0.89 | 5/11 (45) | 1/10 (10) |
| (Acc# X95425) | | | | |
| mRNA for Hrs. (Acc# D84064) | 1.33 | 0.99 | 5/11 (45) | 1/10 (10) |
| mRNA for osteopontin. (Acc# X13694) | 1.49 | 1.15 | 5/11 (45) | 1/10 (10) |
| mRNA for supt5h protein. (Acc# Z50022) | 1.58 | 1.24 | 5/11 (45) | 1/10 (10) |
| Neuroleukin. (Acc# K03515) | 1.70 | 1.11 | 5/11 (45) | 1/10 (10) |
| Platelet-endothelial tetraspan antigen 3. (Acc# | 1.16 | 1.12 | 5/11 (45) | 1/10 (10) |
| U14650) | | | | |
| Protein tyrosine phosphatase (PTPase) (Acc# | 1.92 | 0.97 | 5/11 (45) | 1/10 (10) |
| M25393) | | | | |
| Putative serine/threonine protein kinase PRK | 0.96 | 0.73 | 5/11 (45) | 1/10 (10) |
| (prk) (Acc# U56998) | | | | |
| RP3. (Acc# U02556) | 1.56 | 0.99 | 5/11 (45) | 1/10 (10) |
| Serine threonine kinase 11 (STK11) (Acc# | 1.69 | 1.22 | 5/11 (45) | 1/10 (10) |
| AF035625) | | | | |
| Tat-SF1. (Acc# U76992) | 1.30 | 1.46 | 5/11 (45) | 1/10 (10) |
| Treacher Collins syndrome (TCOF1) (Acc# | 1.85 | 1.26 | 5/11 (45) | 1/10 (10) |
| U40847) | | | | |
| Zinc-finger DNA-binding motifs (IA-1) | 1.07 | 0.87 | 5/11 (45) | 1/10 (10) |
| (Acc# M93119) | | | | |
| Adaptor protein X11beta. (Acc# AF047348) | 2.55 | 0.82 | 6/11 (54) | 2/10 (20) |
| ADP-ribosylation factor 1 (ARF1) (Acc# | 2.02 | 1.04 | 6/11 (54) | 2/10 (20) |
| M36340) | | | | |
| cAMP-dependent protein kinase subunit RII- | 2.73 | 1.21 | 6/11 (54) | 2/10 (20) |
| beta. (Acc# M31158) | | | | |
| Cathepsin D. (Acc# M11233) | 2.09 | 1.11 | 6/11 (54) | 2/10 (20) |
| CL 100 mRNA for protein tyrosine | 4.05 | 1.18 | 6/11 (54) | 2/10 (20) |
| phosphatase. (Acc# # X68277) | | | | |
| COX VIIc gene for subunit VIIc of | 2.77 | 1.40 | 6/11 (54) | 2/10 (20) |
| cytochrome c oxidase (EC 1.9.3.1) (Acc# | | | | |
| U53328) | | | | |
| Dek mRNA. (Acc# L09604) | 2.26 | 1.18 | 6/11 (54) | 2/10 (20) |
| Fau mRNA. (Acc# X65923) | 2.14 | 1.13 | 6/11 (54) | 2/10 (20) |
| H5; and platelet glycoprotein Ib beta chain. | 2.78 | 1.34 | 6/11 (54) | 2/10 (20) |
| (Acc# U59632) | | | | |
| mRNA for 3-hydroxyanthranilic acid | 2.21 | 1.32 | 6/11 (54) | 2/10 (20) |
| dioxygenase. (Acc# Z29481) | | | | |
| mRNA for clathrin coat assembly protein-like. | 2.19 | 1.31 | 6/11 (54) | 2/10 (20) |
| (Acc# D63643) | | | | |
| mRNA for guanidinoacetate N- | 2.14 | 1.20 | 6/11 (54) | 2/10 (20) |
| methyltransferase. (Acc# Z49878) | | | | |
| mRNA for IFP53. (Acc# X62570) | 2.29 | 0.95 | 6/11 (54) | 2/10 (20) |
| mRNA for KIAA0099 gene. (Acc# D43951) | 2.19 | 1.24 | 6/11 (54) | 2/10 (20) |
| mRNA for KIAA0120 gene. (Acc# D21261) | 2.31 | 0.78 | 6/11 (54) | 2/10 (20) |
| mRNA for LDL-receptor related protein. | 2.01 | 0.95 | 6/11 (54) | 2/10 (20) |
| (Acc# X13916) | | | | |
| mRNA for N-acetylglucosaminyltransferase | 2.18 | 1.41 | 6/11 (54) | 2/10 (20) |
| III. (Acc# D13789) | | | | |
| mRNA for nucleoporin-like protein. (Acc# | 2.03 | 1.20 | 6/11 (54) | 2/10 (20) |
| X89478) | | | | |
| mRNA for putative p64 CLCP protein. (Acc# | 2.23 | 1.12 | 6/11 (54) | 2/10 (20) |
| X87689) | | | | |
| mRNA for putative progesterone binding | 2.02 | 1.14 | 6/11 (54) | 2/10 (20) |
| protein. (Acc# Y12711) | | | | |
| N-acetylglucosaminyltransferase I (GlcNAc- | 2.20 | 1.32 | 6/11 (54) | 2/10 (20) |
| TI) (Acc# M55621) | | | | |
| Putative tumor suppressor (LUCA15) (Acc# | 2.89 | 1.38 | 6/11 (54) | 2/10 (20) |
| U17714) | | | | |
| rhoG mRNA for GTPase. (Acc# X61587) | 2.60 | 1.28 | 6/11 (54) | 2/10 (20) |
| Ribosomal protein S9. (Acc# U14971) | 2.29 | 0.75 | 6/11 (54) | 2/10 (20) |
| mRNA for cytochrome c1. (Acc# X06994) | 2.56 | 1.55 | 7/11 (64) | 3/10 (30) |
| alpha-CP1. (Acc# U24223) | 4.09 | 0.94 | 7/11 (64) | 3/10 (30) |
| Glutathione peroxidase (GPX1) (Acc# | 2.54 | 0.93 | 7/11 (64) | 3/10 (30) |
| M21304) | | | | |
| Helix-loop-helix protein (Id-2) (Acc# | 2.10 | 1.36 | 7/11 (64) | 3/10 (30) |
| M97796) | | | | |
| mRNA for BST-2. (Acc# D28137) | 2.21 | 1.59 | 7/11 (64) | 3/10 (30) |
| mRNA for cysteine proteinase inhibitor | 2.43 | 1.44 | 7/11 (64) | 3/10 (30) |
| precursor cystatin C. (Acc# X05607) | | | | |
| mRNA for rat HREV107-like protein. (Acc# | 2.10 | 1.32 | 7/11 (64) | 3/10 (30) |
| X92814) | | | | |
| mRNA PCTAIRE-1 for serine/threonine | 2.13 | 1.03 | 7/11 (64) | 3/10 (30) |
| protein kinase. (Acc#X66363) | | | | |
| UbA52 placental mRNA for ubiquitin-52 | 3.37 | 1.23 | 7/11 (64) | 3/10 (30) |
| amino acid fusion protein. (Acc# X56999) | | | | |
| mRNA for HL23 ribosomal protein | 2.61 | 1.52 | 8/11 (73) | 4/10 (40) |
| homologue. (Acc#X55954) | | | | |
| 26-kDa cell surface protein TAPA-1. (Acc# | 1.05 | 1.01 | 3/11 (27) | 0/10 (0) |
| M33680) | | | | |
| Aac11 (aac11) (Acc# U83867) | 1.54 | 0.84 | 3/11 (27) | 0/10 (0) |
| Alpha SNAP. (Acc# U39412) | 1.09 | 0.87 | 3/11 (27) | 0/10 (0) |
| Arfaptin 2, putative target protein of ADP- | 0.77 | 0.83 | 3/11 (27) | 0/10 (0) |
| ribosylation factor (Acc# U52522) | | | | |
| Argininosuccinate lyase. (Acc# J03058) | 0.99 | 1.04 | 3/11 (27) | 0/10 (0) |
| ArgRS, arginyl-tRNA synthetase. (Acc# | 1.04 | 1.11 | 3/11 (27) | 0/10 (0) |
| S80343) | | | | |
| Brain protein recognized by the sera of | 1.23 | 0.87 | 3/11 (27) | 0/10 (0) |
| patients with paraneoplastic sensory | | | | |
| neuronopathy. (Acc# M52843) | | | | |
| Catechol-O-methyltransferase (COMT) (Acc# | 0.90 | 1.12 | 3/11 (27) | 0/10 (0) |
| M58525) | | | | |
| CDC16Hs. (Acc# U18291) | 1.24 | 0.95 | 3/11 (27) | 0/10 (0) |
| Channel-like integral membrane protein | 1.12 | 1.03 | 3/11 (27) | 0/10 (0) |
| (AQP-1) mRNA, clone AQP-1-1656. (Acc# | | | | |
| U41517) | | | | |
| Chaperonin containing t-complex polypeptide | 1.18 | 0.96 | 3/11 (27) | 0/10 (0) |
| 1, beta subunit (Cctb) (Acc# AF026293) | | | | |
| Chromosome segregation gene homolog CAS. | 1.12 | 1.09 | 3/11 (27) | 0/10 (0) |
| (Acc# U33286) | | | | |
| Cyl mRNA for cytoplasmic tryrosine kinase. | 1.27 | 0.67 | 3/11 (27) | 0/10 (0) |
| (Acc# U32944) | | | | |
| Cysteine protease CPP32 isoform beta. (Acc# | 0.87 | 0.91 | 3/11 (27) | 0/10 (0) |
| J05021) | | | | |
| Dematin (HRD1) (Acc# U03688) | 1.06 | 1.04 | 3/11 (27) | 0/10 (0) |
| EXTR1. (Acc# AB007042) | 1.58 | 1.11 | 3/11 (27) | 0/10 (0) |
| fb19 mRNA. (Acc# Y13247) | 1.48 | 1.03 | 3/11 (27) | 0/10 (0) |
| FKBP-rapamycin associated protein (FRAP) | 0.94 | 0.96 | 3/11 (27) | 0/10 (0) |
| (Acc# L34075) | | | | |
| Gamma SNAP. (Acc# U78107) | 1.15 | 0.94 | 3/11 (27) | 0/10 (0) |
| Gamma-glutamylcysteine synthetase (GCS) | 1.22 | 0.96 | 3/11 (27) | 0/10 (0) |
| (Acc# M90656) | | | | |
| Glutamate receptor flop isoform (GluR3-flop) | 1.39 | 0.96 | 3/11 (27) | 0/10 (0) |
| (Acc# U10302) | | | | |
| GS1 (protein of unknown function) (Acc# | 1.59 | 1.04 | 3/11 (27) | 0/10 (0) |
| M86934) | | | | |
| GST1-Hs mRNA for GTP-binding protein. | 1.02 | 0.83 | 3/11 (27) | 0/10 (0) |
| (Acc# X17644) | | | | |
| Heme oxygenase-2. (Acc# S34389) | 1.43 | 0.99 | 3/11 (27) | 0/10 (0) |
| hSIAH1. (Acc# U76247) | 1.23 | 1.18 | 3/11 (27) | 0/10 (0) |
| Jun activation domain binding protein. (Acc# | 1.22 | 0.96 | 3/11 (27) | 0/10 (0) |
| U65928) | | | | |
| Membrane glycoprotein 4F2 antigen heavy | 1.48 | 1.12 | 3/11 (27) | 0/10 (0) |
| chain mRNA. (Acc# J02939) | | | | |
| mRNA for ARP1 protein. (Acc# X91504) | 1.24 | 0.97 | 3/11 (27) | 0/10 (0) |
| mRNA for ATP-citrate lyase. (Acc# X64330) | 0.94 | 1.05 | 3/11 (27) | 0/10 (0) |
| mRNA for carboxypeptidase E (EC 3.4.17.10) | 1.66 | 1.29 | 3/11 (27) | 0/10 (0) |
| (Acc# X51405) | | | | |
| mRNA for collagen VI alpha-2 N-terminal | 0.99 | 1.05 | 3/11 (27) | 0/10 (0) |
| globular domain. (Acc# X15881) | | | | |
| mRNA for E14 protein (Acc# 97186) | 0.93 | 0.86 | 3/11 (27) | 0/10 (0) |
| mRNA for GS3955 (Acc# D87119) | 1.13 | 1.23 | 3/11 (27) | 0/10 (0) |
| mRNA for IgG Fc binding protein (Acc# | 0.61 | 1.06 | 3/11 (27) | 0/10 (0) |
| D84239) | | | | |
| mRNA for KIAA0132 gene (Acc# D50922) | 1.17 | 0.94 | 3/11 (27) | 0/10 (0) |
| mRNA for KIAA0174 gene (Acc# D79996) | 1.25 | 1.11 | 3/11 (27) | 0/10 (0) |
| mRNA for KIAA0255 gene (Acc# D87444) | 0.92 | 1.26 | 3/11 (27) | 0/10 (0) |
| mRNA for KIAA0262 gene (Acc# D87451) | 1.13 | 0.88 | 3/11 (27) | 0/10 (0) |
| mRNA for KIAA0274 gene (Acc# D87464) | 1.37 | 1.15 | 3/11 (27) | 0/10 (0) |
| mRNA for KIAA0343 gene (Acc# | 0.98 | 1.21 | 3/11 (27) | 0/10 (0) |
| AB002341) | | | | |
| mRNA for macropain subunit zeta (Acc# | 1.61 | 1.20 | 3/11 (27) | 0/10 (0) |
| X61970) | | | | |
| mRNA for Mr 110,000 antigen (Acc# | 1.24 | 1.12 | 3/11 (27) | 0/10 (0) |
| D64154) | | | | |
| mRNA for muscle specific enolase (MSE) | 0.91 | 0.64 | 3/11 (27) | 0/10 (0) |
| (EC 4.2.1.11) (Acc# X51957) | | | | |
| mRNA for myosin heavy chain 12 (Acc# | 0.98 | 0.90 | 3/11 (27) | 0/10 (0) |
| Y07759) | | | | |
| mRNA for neuropathy target esterase (Acc# | 1.16 | 1.11 | 3/11 (27) | 0/10 (0) |
| AJ004832) | | | | |
| mRNA for phenylalkylamine binding protein | 1.24 | 1.07 | 3/11 (27) | 0/10 (0) |
| (Acc# Z37986) | | | | |
| mRNA for phosphorylase-kinase, beta subunit | 0.95 | 0.80 | 3/11 (27) | 0/10 (0) |
| (Acc# X84908) | | | | |
| mRNA for protein phosphatase 2C gamma | 1.37 | 1.33 | 3/11 (27) | 0/10 (0) |
| (Acc# Y13936) | | | | |
| mRNA for TPRDIII (Acc# D84296) | 1.04 | 0.74 | 3/11 (27) | 0/10 (0) |
| mRNA for tre oncogene (clone 210) (Acc# | 0.96 | 0.79 | 3/11 (27) | 0/10 (0) |
| X63546) | | | | |
| mRNA for U3 snoRNP associated 55 kDa | 0.73 | 1.20 | 3/11 (27) | 0/10 (0) |
| protein (Acc# AJ001340) | | | | |
| mRNA for villin-like protein (Acc# D88154) | 1.02 | 1.08 | 3/11 (27) | 0/10 (0) |
| MSH3 gene (Acc# J04810) | 0.98 | 0.75 | 3/11 (27) | 0/10 (0) |
| Myotubularin (MTM1) (Acc# U46024) | 1.13 | 0.92 | 3/11 (27) | 0/10 (0) |
| Natural killer cell enhancing factor (NKEFB) | 1.35 | 0.79 | 3/11 (27) | 0/10 (0) |
| (Acc# L19185) | | | | |
| Neurogranin (RC3) (Acc# U89165) | 1.17 | 1.07 | 3/11 (27) | 0/10 (0) |
| Neuron-specific gamma-2 enolase (Acc# | 1.17 | 1.20 | 3/11 (27) | 0/10 (0) |
| M22349) | | | | |
| NF-ATc (Acc# U08015) | 1.03 | 1.41 | 3/11 (27) | 0/10 (0) |
| N-myristoyltransferase 2 (Acc# AF043325) | 1.20 | 0.99 | 3/11 (27) | 0/10 (0) |
| Nuclear antigen H731-like protein (Acc# | 1.24 | 1.04 | 3/11 (27) | 0/10 (0) |
| U96628) | | | | |
| Nuclear factor I-X (Acc# L31881) | 0.96 | 1.12 | 3/11 (27) | 0/10 (0) |
| PKY protein kinase. (Acc# AF004849) | 1.09 | 1.12 | 3/11 (27) | 0/10 (0) |
| Plasma membrane calcium ATPase isoform | 1.52 | 1.26 | 3/11 (27) | 0/10 (0) |
| 3x/b (Acc# U60414) | | | | |
| PROS-27 mRNA (Acc# X59417) | 1.71 | 0.84 | 3/11 (27) | 0/10 (0) |
| Protein tyrosine kinase (Acc# M59371) | 0.90 | 0.92 | 3/11 (27) | 0/10 (0) |
| Protein-tyrosine phosphatase (PTPase MEG2) | 0.73 | 0.99 | 3/11 (27) | 0/10 (0) |
| (Acc# M83738) | | | | |
| Replication protein A 70 kDa subunit mRNA | 1.30 | 0.95 | 3/11 (27) | 0/10 (0) |
| complete cds (Acc# M63488) | | | | |
| SCG10, neuron-specific growth-associated | 0.96 | 0.78 | 3/11 (27) | 0/10 (0) |
| protein/stathmin homolog (Acc# S82024) | | | | |
| Serine kinase (Acc# U09564) | 1.18 | 0.97 | 3/11 (27) | 0/10 (0) |
| SHB mRNA (Acc# X75342) | 0.93 | 1.07 | 3/11 (27) | 0/10 (0) |
| Smg GDS-associated protein SMAP (Acc# | 1.29 | 1.12 | 3/11 (27) | 0/10 (0) |
| U59919) | | | | |
| SnRNP core protein Sm D3 (Acc# U15009) | 0.69 | 0.84 | 3/11 (27) | 0/10 (0) |
| Spermidine synthase (Acc# M34338) | 1.25 | 1.25 | 3/11 (27) | 0/10 (0) |
| subunit of coatomer complex (Acc# X70476) | 1.69 | 1.01 | 3/11 (27) | 0/10 (0) |
| SWI/SNF complex 60 KDa subunit (BAF60c) | 1.69 | 1.07 | 3/11 (27) | 0/10 (0) |
| (Acc# U66619) | | | | |
| tax1-binding protein TXBP151 (Acc# | 1.05 | 1.01 | 3/11 (27) | 0/10 (0) |
| U33821) | | | | |
| 20-kDa myosin light chain (MLC-2) (Acc# | 1.40 | 1.02 | 4/11 (36) | 1/10 (10) |
| J02854) | | | | |
| alpha-2 collagen type VI-a′ gene, exon 1 | 1.20 | 0.82 | 4/11 (36) | 1/10 (10) |
| (Acc# M34573) | | | | |
| Amyloid beta-peptide binding protein (ERAB) | 1.51 | 1.22 | 4/11 (36) | 1/10 (10) |
| (Acc# U96132) | | | | |
| Amyloid precursor-like protein 1 (Acc# | 1.34 | 1.21 | 4/11 (36) | 1/10 (10) |
| U48437) | | | | |
| Antigen CD36 (clone 13) (Acc# M98398) | 1.32 | 1.26 | 4/11 (36) | 1/10 (10) |
| ARL1 (Acc# L28997) | 1.71 | 0.86 | 4/11 (36) | 1/10 (10) |
| BRCA1 associated protein 1 (BAP1) (Acc# | 0.83 | 1.00 | 4/11 (36) | 1/10 (10) |
| AF045581) | | | | |
| Ca2-activated neutral protease large subunit | 1.32 | 1.25 | 4/11 (36) | 1/10 (10) |
| (CANP) (Acc# M23254) | | | | |
| Cadherin-associated protein-related (cap-r) | 1.61 | 1.07 | 4/11 (36) | 1/10 (10) |
| (Acc# M94151) | | | | |
| Cathepsin B proteinase (Acc# M14221) | 1.54 | 0.95 | 4/11 (36) | 1/10 (10) |
| Clone 24444 RaP2 interacting protein 8 | 1.45 | 1.09 | 4/11 (36) | 1/10 (10) |
| (RPIP8) (Acc# AF055026) | | | | |
| Creatine kinase B (Acc# U34051) | 1.51 | 0.87 | 4/11 (36) | 1/10 (10) |
| Creatine kinase M (Acc# U79269) | 1.08 | 1.05 | 4/11 (36) | 1/10 (10) |
| Cyclin G (Acc# U62800) | 1.71 | 1.02 | 4/11 (36) | 1/10 (10) |
| Cytoplasmic dynein light chain 1 (hdlc1) | 1.66 | 0.94 | 4/11 (36) | 1/10 (10) |
| (Acc# AF035119) | | | | |
| Dynamin (DNM) (Acc# S72422) | 1.06 | 1.04 | 4/11 (36) | 1/10 (10) |
| Endonuclease III homolog. (Acc# U94836) | 1.33 | 1.32 | 4/11 (36) | 1/10 (10) |
| Factor KBF1 (Acc# M55643) | 1.82 | 1.11 | 4/11 (36) | 1/10 (10) |
| Ferritin heavy chain (Acc# L20941) | 1.15 | 0.88 | 4/11 (36) | 1/10 (10) |
| flotillin-1 (Acc# AF089750) | 1.62 | 1.30 | 4/11 (36) | 1/10 (10) |
| Hepatitis B virus X interacting protein (XIP) | 0.87 | 0.86 | 4/11 (36) | 1/10 (10) |
| (Acc# AF029890) | | | | |
| hLRp105 mRNA for LDL receptor related | 0.70 | 0.97 | 4/11 (36) | 1/10 (10) |
| protein 105 (Acc# AB009462) | | | | |
| HMG-Y protein isoform mRNA (HMGI | 1.44 | 0.98 | 4/11 (36) | 1/10 (10) |
| gene), clone 11D (Acc# M23618) | | | | |
| Importin beta subunit (Acc# L38951) | 1.07 | 0.92 | 4/11 (36) | 1/10 (10) |
| jun-D mRNA for JUN-D protein (Acc# | 1.24 | 0.83 | 4/11 (36) | 1/10 (10) |
| X51346) | | | | |
| Lymphoid nuclear protein (LAF-4) (Acc# | 1.43 | 1.40 | 4/11 (36) | 1/10 (10) |
| U34360) | | | | |
| Mad4 homolog (Mad4) (Acc# AF040963) | 1.10 | 0.96 | 4/11 (36) | 1/10 (10) |
| Malate dehydrogenase precursor (MDH) | 1.71 | 1.04 | 4/11 (36) | 1/10 (10) |
| mRNA, nuclear gene encoding mitochondrial | | | | |
| protein (Acc# AF047470) | | | | |
| Microtubule-associated protein 1A (MAP1A) | 1.49 | 1.16 | 4/11 (36) | 1/10 (10) |
| (Acc# U14577) | | | | |
| Microtubule-associated protein 2 (MAP2) | 1.75 | 1.06 | 4/11 (36) | 1/10 (10) |
| (Acc# U10828) | | | | |
| mRNA for actin binding protein p57 (Acc# | 1.42 | 0.94 | 4/11 (36) | 1/10 (10) |
| D44497) | | | | |
| mRNA for eIF-2B alpha subunit (Acc# | 1.08 | 1.21 | 4/11 (36) | 1/10 (10) |
| X95648) | | | | |
| mRNA for Eph-family protein (Acc# | 1.44 | 0.97 | 4/11 (36) | 1/10 (10) |
| D83492) | | | | |
| mRNA for GABA-A receptor, gamma 2 | 1.41 | 0.95 | 4/11 (36) | 1/10 (10) |
| subunit. (Acc# X15376) | | | | |
| mRNA for GEF-2 protein (Acc# AJ010569) | 1.47 | 0.93 | 4/11 (36) | 1/10 (10) |
| mRNA for integrin alpha 6 (Acc# X53586) | 1.70 | 1.39 | 4/11 (36) | 1/10 (10) |
| mRNA for KIAA0144 gene (Acc# D63478) | 1.16 | 0.94 | 4/11 (36) | 1/10 (10) |
| mRNA for KIAA0266 gene. (Acc# D87455) | 1.90 | 1.10 | 4/11 (36) | 1/10 (10) |
| mRNA for long-chain acyl-CoA synthetase | 1.02 | 1.16 | 4/11 (36) | 1/10 (10) |
| (Acc# D10040) | | | | |
| mRNA for membrane cofactor protein (Acc# | 1.36 | 1.18 | 4/11 (36) | 1/10 (10) |
| Y00651) | | | | |
| mRNA for metabotropic glutamate receptor | 1.29 | 1.06 | 4/11 (36) | 1/10 (10) |
| subtype 5a (Acc# D28538) | | | | |
| mRNA for mitochondrial isocitrate | 1.61 | 1.42 | 4/11 (36) | 1/10 (10) |
| dehydrogenase (NADP+) (Acc# X69433) | | | | |
| mRNA for N-acetylglucosamide-(beta 1-4)- | 1.16 | 1.19 | 4/11 (36) | 1/10 (10) |
| galactosyltransferase (Acc# X13223) | | | | |
| mRNA for neurotrypsin (Acc# AJ001531) | 1.30 | 1.41 | 4/11 (36) | 1/10 (10) |
| mRNA for p40 (Acc# Y11395) | 1.90 | 1.21 | 4/11 (36) | 1/10 (10) |
| mRNA for peroxisome assembly factor-2 | 1.29 | 1.38 | 4/11 (36) | 1/10 (10) |
| (Acc# D83703) | | | | |
| mRNA for PTB-associated splicing factor | 1.28 | 1.29 | 4/11 (36) | 1/10 (10) |
| (Acc# X70944) | | | | |
| mRNA for succinyl CoA synthetase (Acc# | 1.65 | 1.56 | 4/11 (36) | 1/10 (10) |
| Z68204) | | | | |
| mRNA for thioredoxin reductase (Acc# | 1.10 | 1.08 | 4/11 (36) | 1/10 (10) |
| X91247) | | | | |
| mRNA for type 1 inositol 1,4,5-trisphosphate | 1.55 | 1.17 | 4/11 (36) | 1/10 (10) |
| receptor (Acc# D26070) | | | | |
| mRNA for ZNF74 protein (Acc# X92715) | 0.84 | 0.68 | 4/11 (36) | 1/10 (10) |
| NAP (nucleosome assembly protein) (Acc# | 1.61 | 1.13 | 4/11 (36) | 1/10 (10) |
| M86667) | | | | |
| Nidogen (Acc# M30269) | 1.34 | 1.06 | 4/11 (36) | 1/10 (10) |
| Nuclear receptor co-repressor N-CoR (Acc# | 1.41 | 0.88 | 4/11 (36) | 1/10 (10) |
| AF044209) | | | | |
| Partial C1 mRNA (Acc# X78817) | 1.70 | 0.99 | 4/11 (36) | 1/10 (10) |
| Phosphomevalonate kinase (Acc# L77213) | 1.39 | 0.77 | 4/11 (36) | 1/10 (10) |
| PWP2H protein (Acc# U53346) | 1.64 | 1.22 | 4/11 (36) | 1/10 (10) |
| Rearranged mRNA for glutamine synthase | 1.27 | 1.32 | 4/11 (36) | 1/10 (10) |
| (Acc# X59834) | | | | |
| Ser-thr protein kinase PK428 (Acc# U59305) | 1.71 | 1.12 | 4/11 (36) | 1/10 (10) |
| SPF31 (SPF31) (Acc# AF083190) | 1.79 | 1.05 | 4/11 (36) | 1/10 (10) |
| Sterol carrier protein X/sterol carrier protein 2 | 1.01 | 1.28 | 4/11 (36) | 1/10 (10) |
| (Acc# M75883) | | | | |
| TLS/CHOP, hybrid gene (Acc# S62138) | 1.64 | 0.79 | 4/11 (36) | 1/10 (10) |
| Transcriptional regulator homolog RPD3 | 1.56 | 1.14 | 4/11 (36) | 1/10 (10) |
| (Acc# U31814) | | | | |
| Uridine diphosphoglucose pyrophosphorylase. | 1.41 | 1.19 | 4/11 (36) | 1/10 (10) |
| YPT3 mRNA (Acc# U27460) | 1.61 | 1.34 | 4/11 (36) | 1/10 (10) |
| ADP-ribosylation factor-like protein 2 (ARL2) | 1.00 | 1.02 | 5/11 (45) | 2/10 (20) |
| (Acc# L13687) | | | | |
| Arginine-rich nuclear protein (Acc# M74002) | 0.95 | 1.02 | 5/11 (45) | 2/10 (20) |
| Arp2/3 protein complex subunit p16-Arc | 1.73 | 1.58 | 5/11 (45) | 2/10 (20) |
| (ARC16) (Acc# AF006088) | | | | |
| biliverdin-IX alpha reductase (Acc# U34877) | 1.21 | 0.96 | 5/11 (45) | 2/10 (20) |
| Cellular growth-regulating protein (Acc# | 1.95 | 1.22 | 5/11 (45) | 2/10 (20) |
| L10844) | | | | |
| CLP (Acc# L54057) | 1.65 | 1.21 | 5/11 (45) | 2/10 (20) |
| DNF1552 (lung) (Acc# M31222) | 1.16 | 0.88 | 5/11 (45) | 2/10 (20) |
| e12 protein (E2A) (Acc# U13261) | 1.80 | 1.23 | 5/11 (45) | 2/10 (20) |
| Integral membrane protein CII-3 mRNA, | 1.22 | 0.76 | 5/11 (45) | 2/10 (20) |
| nuclear gene encoding mitochondrial protein | | | | |
| (Acc# U57877) | | | | |
| K+ channel beta 2 subunit (Acc# U33429) | 1.03 | 1.38 | 5/11 (45) | 2/10 (20) |
| Lysosomal membrane glycoprotein-2 | 1.67 | 1.30 | 5/11 (45) | 2/10 (20) |
| (LAMP2) (Acc# J04183) | | | | |
| Mitochondrial ATP synthase subunit 9, P3 | 1.73 | 0.98 | 5/11 (45) | 2/10 (20) |
| gene copy, mRNA, nuclear gene encoding | | | | |
| mitochondrial protein (Acc# U09813) | | | | |
| rnRNA for 2.19 gene (Acc# X87193) | 1.67 | 1.01 | 5/11 (45) | 2/10 (20) |
| mRNA for alpha-1,3-fucosyl-transferase | 1.08 | 1.09 | 5/11 (45) | 2/10 (20) |
| (Acc# X78031) | | | | |
| mRNA for collagen VI alpha-1 N-terminal | 1.75 | 1.37 | 5/11 (45) | 2/10 (20) |
| globular domain (Acc# X15879) | | | | |
| mRNA for eukaryotic initiation factor 4AI | 1.98 | 1.32 | 5/11 (45) | 2/10 (20) |
| (Acc# D12686) | | | | |
| mRNA for FAN protein (Acc# X96586) | 1.79 | 1.32 | 5/11 (45) | 2/10 (20) |
| mRNA for glia maturation factor (Acc# | 1.99 | 1.05 | 5/11 (45) | 2/10 (20) |
| AB001106) | | | | |
| mRNA for H+-ATP synthase subunit b (Acc# | 1.37 | 1.22 | 5/11 (45) | 2/10 (20) |
| X60221) | | | | |
| mRNA for KIAA0106 gene (Acc# D14662) | 1.49 | 0.79 | 5/11 (45) | 2/10 (20) |
| mRNA for KIAA0111 gene (Acc# D21853) | 1.77 | 1.08 | 5/11 (45) | 2/10 (20) |
| mRNA for mitochondrial short-chain enoyl- | 1.72 | 1.41 | 5/11 (45) | 2/10 (20) |
| CoA hydratase (Acc# D13900) | | | | |
| mRNA for TGF-betaIIR alpha (Acc# | 1.76 | 0.99 | 5/11 (45) | 2/10 (20) |
| D50683) | | | | |
| mRNA for TGIF protein (Acc# X89750) | 1.64 | 0.89 | 5/11 (45) | 2/10 (20) |
| mRNA for vacuolar proton-ATPase subunit | 1.93 | 1.35 | 5/11 (45) | 2/10 (20) |
| M9.2 (Acc# Y15286) | | | | |
| Mutated in multiple advanced cancers protein | 1.78 | 0.91 | 5/11 (45) | 2/10 (20) |
| (MMAC1) (Acc# U92436) | | | | |
| Neural cell adhesion molecule (Acc# | 1.92 | 1.05 | 5/11 (45) | 2/10 (20) |
| AF005070) | | | | |
| Protein tyrosine phosphatase (TEP1) (Acc# | 1.76 | 1.15 | 5/11 (45) | 2/10 (20) |
| U96180) | | | | |
| Putative NDP kinase (nm23-H2S) (Acc# | 1.94 | 1.28 | 5/11 (45) | 2/10 (20) |
| M36981) | | | | |
| Ribosomal protein L12 (Acc# L06505) | 1.99 | 1.23 | 5/11 (45) | 2/10 (20) |
| Splicing factor (CC1.3) (Acc# L10910) | 1.42 | 1.13 | 5/11 (45) | 2/10 (20) |
| tra1 mRNA forhomologue of murine tumor | 1.15 | 1.08 | 5/11 (45) | 2/10 (20) |
| rejection antigen gp96 (Acc# X15187) | | | | |
| Transmembrane protein Jagged 1 (HJ1) | 1.76 | 0.90 | 5/11 (45) | 2/10 (20) |
| (Acc# AF028593) | | | | |
| Tristetraproline (TTP) (Acc# M63625) | 1.16 | 1.27 | 5/11 (45) | 2/10 (20) |

Underexpressed Genes

| | UC | NonIBD | UC < 5 | OI < 5 |
|---|---|---|---|---|
| Gene ID | Median | Median | 11 | 10 |
| G protein gamma-4 subunit (Acc# U31382 | 0.39 | 0.99 | 7/11 (64) | 0/10 (0) |
| Retroviral receptor mRNA (Acc# X59155) | 0.34 | 0.94 | 8/11 (73) | 1/10 (10) |
| mRNA for protein kinase, PKX1 (Acc# X85545) | 0.37 | 1.16 | 8/11 (73) | 1/10 (10) |
| mRNA for KIAA0235 gene (Acc# D87078) | 0.39 | 1.00 | 8/11 (73) | 2/10 (20) |
| Caco-2 oligopeptide transporter (PEPT1) (Acc# | 0.21 | 0.58 | 9/11 (82) | 3/10 (30) |
| AF043233) | | | | |
| mRNA for DCRA (Acc# D87343) | 0.57 | 0.85 | 5/11 (45) | 0/10 (0) |
| Achaete scute homologous protein (ASH1) (Acc# | 0.54 | 0.70 | 5/11 (45) | 0/10 (0) |
| L08424) | | | | |
| Podocalyxin-like protein (Acc# U97519) | 0.61 | 0.95 | 5/11 (45) | 0/10 (0) |
| mRNA for KIAA0210 gene (Acc# D86965) | 0.69 | 0.92 | 5/11 (45) | 0/10 (0) |
| Retinoid X receptor beta (RXR-beta) (Acc# | 0.51 | 0.93 | 5/11 (45) | 0/10 (0) |
| M84820) | | | | |
| mRNA for CASH beta protein (Acc# Y14040) | 0.46 | 0.86 | 6/11 (55) | 1/10 (10) |
| P2x1 receptor (Acc# U45448) | 0.48 | 1.08 | 6/11 (55) | 1/10 (10) |
| mRNA for NRAMP2 (Acc# AB004857) | 0.44 | 0.96 | 6/11 (55) | 1/10 (10) |
| Stratum corneum chymotryptic enzyme (Acc# | 0.48 | 1.04 | 6/11 (55) | 1/10 (10) |
| L33404) | | | | |
| mRNA for protein kinase C mu (Acc# X75756) | 0.39 | 0.81 | 6/11 (55) | 1/10 (10) |
| Nucleolar autoantigen No55 (Acc# U47621) | 0.40 | 0.79 | 7/11 (64) | 2/10 (20) |
| Microtubule-associated protein 4 (Acc# M64571) | 0.44 | 1.06 | 7/11 (64) | 2/10 (20) |
| Microsomal aldehyde dehydrogenase (ALD10) | 0.43 | 0.75 | 7/11 (64) | 2/10 (20) |
| (Acc# U046689) | | | | |
| TTAGGG repeat binding factor 2 (hTRF2) (Acc# | 0.38 | 1.19 | 7/11 (64) | 2/10 (20) |
| AF002999) | | | | |
| Neuronal kinesin heavy chain (Acc# U06698) | 0.43 | 0.98 | 7/11 (64) | 2/10 (20) |
| Apoptotic protease activating factor 1 (Apaf-1) | 0.44 | 1.05 | 7/11 (64) | 2/10 (20) |
| (Acc# AF012363) | | | | |
| G protein-coupled receptor (STRL22) (Acc# | 0.32 | 1.37 | 7/11 (64) | 2/10 (20) |
| U68030) | | | | |
| Cell matrix adhesion regulator variant (CMAR) | 0.38 | 0.74 | 7/11 (64) | 2/10 (20) |
| (Acc# AFO34795) | | | | |
| interleukin-1 receptor-associated kinase (IRAK) | 0.42 | 0.75 | 7/11 (64) | 2/10 (20) |
| (Acc# L76191) | | | | |
| Proline and glutamic acid rich nuclear protein | 0.48 | 1.02 | 7/11 (64) | 2/10 (20) |
| isoform (Acc# U88154) | | | | |
| Profilaggrin mRNA, 5′ end (Acc# M60499) | 0.36 | 0.74 | 8/11 (73) | 3/10 (30) |
| Synaptotagmin (Acc# M55047) | 0.29 | 0.73 | 8/11 (73) | 3/10 (30) |
| KIAA0415 (Acc# AB007875) | 0.37 | 0.95 | 8/11 (73) | 3/10 (30) |
| RBQ-3 mRNA (Acc# X85134) | 0.29 | 0.90 | 9/11 (82) | 4/10 (40) |
| mRNA for TRAF5 (Acc# AB000509) | 0.38 | 0.84 | 9/11 (82) | 4/10 (40) |
| mRNA for KIAA0168 gene (Acc# D79990) | 1.97 | 0.81 | 4/11 (36) | 0/10 (0) |
| Gamma SNAP (Acc# U78107) | 1.15 | 0.94 | 4/11 (36) | 0/10 (0) |
| mRNA for high mobility group protein HMG2a | 0.60 | 0.72 | 4/11 (36) | 0/10 (0) |
| (Acc# Y10043) | | | | |
| mRNA for b4 integrin interactor (Acc# Y11435) | 0.67 | 0.91 | 4/11 (36) | 0/10 (0) |
| DNA-dependent protein kinase catalytic subunit | 0.82 | 0.89 | 4/11 (36) | 0/10 (0) |
| (DNA-PKcs) (Acc# U31850) | | | | |
| mRNA for KIAA0307 gene (Acc# AB002305) | 1.26 | 0.97 | 4/11 (36) | 0/10 (0) |
| cytohesin-2 (Acc# AF000982) | 0.68 | 0.95 | 4/11 (36) | 0/10 (0) |
| Keratin 10 type I intermediate filament (KRT10) | 0.68 | 0.86 | 4/11 (36) | 0/10 (0) |
| (Acc# J04029) | | | | |
| mRNA for OTK27 (Acc# D50420) | 0.62 | 0.77 | 4/11 (36) | 0/10 (0) |
| mRNA for plasma gelsolin (Acc# X04412) | 0.69 | 0.97 | 4/11 (36) | 0/10 (0) |
| mRNA for protein D123 (Acc# D14878) | 0.82 | 1.17 | 4/11 (36) | 0/10 (0) |
| PKC alpha mRNA for protein kinase C alpha | 0.68 | 0.88 | 4/11 (36) | 0/10 (0) |
| (Acc# X52479) | | | | |
| mRNA for XP-C repair complementing protein | 0.62 | 0.91 | 4/11 (36) | 0/10 (0) |
| (p58/HHR23B) (Acc# D21090) | | | | |
| Ankyrin G (ANK-3) (Acc# U13616) | 0.67 | 1.08 | 4/11 (36) | 0/10 (0) |
| mRNA for brain ankyrin (brank-2) (Acc# | 0.66 | 0.90 | 4/11 (36) | 0/10 (0) |
| X56958) | | | | |
| Notch3 (NOTCH3) (Acc# U97669) | 0.67 | 1.04 | 4/11 (36) | 0/10 (0) |
| Aspartyl beta-hydroxylase (Acc# U03109) | 0.90 | 1.23 | 4/11 (36) | 0/10 (0) |
| Cystatin B (Acc# S70154) | 0.83 | 0.81 | 5/11 (45) | 1/10 (10) |
| Lysyl oxidase-related protein (WS9-14) (Acc# | 0.76 | 0.96 | 5/11 (45) | 1/10 (10) |
| U89942) | | | | |
| aminoacylase-1 (ACY1) (Acc# L07548) | 0.69 | 0.87 | 5/11 (45) | 1/10 (10) |
| Thymidine kinase (Acc# K02581) | 0.79 | 0.96 | 5/11 (45) | 1/10 (10) |
| 26S protease subunit 5b, 50 kda subunit (Acc# | 0.57 | 0.88 | 5/11 (45) | 1/10 (10) |
| S79862) | | | | |
| ADP-ribosylation factor 3 (Acc# M74491) | 0.87 | 0.84 | 5/11 (45) | 1/10 (10) |
| Neuronal DHP-sensitive, voltage-dependent, | 0.59 | 0.82 | 5/11 (45) | 1/10 (10) |
| calcium channel alpha-1D subunit (Acc# | | | | |
| M76558) | | | | |
| T cell leukemia LERK-2 (EPLG2) (Acc# | 0.75 | 0.88 | 5/11 (45) | 1/10 (10) |
| U09303) | | | | |
| Ubiquitin conjugating enzyme G2 (UBE2G2) | 0.52 | 1.03 | 5/11 (45) | 1/10 (10) |
| (Acc# AF032456) | | | | |
| mRNA for KIAA0054 gene (Acc# D29677) | 0.89 | 0.95 | 5/11 (45) | 1/10 (10) |
| (Acc# D21267) | 0.50 | 0.88 | 6/11 (55) | 2/10 (20) |
| 2,3-oxidosqualene-lanosterol cyclase (Acc# | 0.50 | 0.94 | 6/11 (55) | 2/10 (20) |
| U22526) | | | | |
| Alpha-tubulin (Acc# K00558) | 0.42 | 0.86 | 6/11 (55) | 2/10 (20) |
| Insulin receptor substrate-1 (Acc# S62539) | 0.41 | 0.67 | 6/11 (55) | 2/10 (20) |
| Klotho (Acc# AB005142) | 0.46 | 0.90 | 6/11 (55) | 2/10 (20) |
| Alpha 2 delta calcium channel subunit isoform II | 0.47 | 0.87 | 6/11 (55) | 2/10 (20) |
| (Acc# AF042793) | | | | |
| CD36 antigen (Acc# M24795) | 0.39 | 1.24 | 6/11 (55) | 2/10 (20) |
| HMG-I protein isoform mRNA (HMGI gene), | 0.46 | 0.91 | 6/11 (55) | 2/10 (20) |
| clone 7C (Acc# M23614) | | | | |
| Histidine-rich calcium binding protein (HRC) | 0.44 | 0.85 | 6/11 (55) | 2/10 (20) |
| (Acc# M60052) | | | | |
| ZNF43 mRNA (Acc# X59244) | 0.38 | 0.83 | 6/11 (55) | 2/10 (20) |
| cAMP phosphodiesterase (Pde7A2) (Acc# | 0.47 | 0.97 | 6/11 (55) | 2/10 (20) |
| U67932) | | | | |
| Clone iota unknown protein (Acc# AF001435) | 0.39 | 1.09 | 6/11 (55) | 2/10 (20) |
| fibulin-1D (Acc# U01244) | 0.47 | 1.03 | 6/11 (55) | 2/10 (20) |
| Brain-expressed HHCPA78 homolog (Acc# | 0.35 | 0.92 | 7/11 (64) | 3/10 (30) |
| S73591) | | | | |
| (oct-6) (Acc# L23494) | 0.38 | 0.87 | 7/11 (64) | 3/10 (30) |
| Carnitine palmitoyltransferase II precursor (CPT1) | 0.36 | 1.01 | 7/11 (64) | 3/10 (30) |
| (Acc# U09648) | | | | |
| FGFR2 mRNA (Acc# Z71929) | 0.26 | 0.76 | 7/11 (64) | 3/10 (30) |
| TLS, translocated in liposarcoma (Acc# S62138) | 0.31 | 0.69 | 7/11 (64) | 3/10 (30) |
| Tumor necrosis factor superfamily member | 0.15 | 0.85 | 7/11 (64) | 3/10 (30) |
| LIGHT (Acc# AF036581) | | | | |
| mRNA for desmocollin type 1 (Acc# X72925) | 0.43 | 1.02 | 7/11 (64) | 3/10 (30) |
| NADH-ubiquinone oxidoreductase subunit CI-B14 | 0.43 | 0.60 | 7/11 (64) | 3/10 (30) |
| (Acc# AF047182) | | | | |
| IGF-I mRNA for insulin-like growth factor I | 0.37 | 0.84 | 8/11 (73) | 4/10 (40) |
| (Acc# X57025) | | | | |
| mRNA for KIAA0331 gene (Acc# AB002329) | 0.27 | 0.84 | 8/11 (73) | 4/10 (40) |
| Arfaptin 2, putative target protein of ADP- | 0.77 | 0.83 | 3/11 (27) | 0/10 (0) |
| ribosylation factor, (Acc# U52522) | | | | |
| mRNA for collagen VI alpha-2 N-terminal | 0.99 | 1.05 | 3/11 (27) | 0/10 (0) |
| globular domain (Acc# X15881) | | | | |
| Nuclear antigen H731-like protein (Acc# | 1.24 | 1.04 | 3/11 (27) | 0/10 (0) |
| U96628) | | | | |
| Antigen CD36 (clone 13) (Acc# M98398) | 1.32 | 1.26 | 3/11 (27) | 0/10 (0) |
| mRNA for thrombospondin-4 (Acc# Z19585) | 0.85 | 1.12 | 3/11 (27) | 0/10 (0) |
| Surface antigen (Acc# M60922) | 0.87 | 0.73 | 3/11 (27) | 0/10 (0) |
| Translation repressor NAT1 (Acc# U76111) | 0.82 | 1.03 | 3/11 (27) | 0/10 (0) |
| Zinc finger protein (Acc# U68536) | 0.79 | 0.86 | 3/11 (27) | 0/10 (0) |
| alpha-2-macroglobulin receptor-associated protein | 0.85 | 1.18 | 3/11 (27) | 0/10 (0) |
| (Acc# M63959) | | | | |
| Associated microfibrillar protein (Acc# U04209) | 0.79 | 0.75 | 3/11 (27) | 0/10 (0) |
| cdc2-related protein kinase (CHED) (Acc# | 0.74 | 0.80 | 3/11 (27) | 0/10 (0) |
| M80629) | | | | |
| cDNA for dihydroxyacetone phosphate | 0.81 | 0.92 | 3/11 (27) | 0/10 (0) |
| acyltransferase (DAP-AT) (Acc# AJ002190) | | | | |
| DNA-binding protein (CROC-1A) (Acc# L07807) | 0.90 | 0.93 | 3/11 (27) | 0/10 (0) |
| DNA-binding protein (CROC-1B) (Acc# U50733) | 0.86 | 0.76 | 3/11 (27) | 0/10 (0) |
| Guanine nucleotide exchange factor p532 (Acc# | 0.84 | 0.92 | 3/11 (27) | 0/10 (0) |
| U50078) | | | | |
| Kinesin light chain (Acc# L04733) | 0.92 | 0.92 | 3/11 (27) | 0/10 (0) |
| mRNA for emerin. (Acc# X82434) | 0.86 | 1.06 | 3/11 (27) | 0/10 (0) |
| mRNA for transcriptional intermediary factor 2 | 1.04 | 1.07 | 3/11 (27) | 0/10 (0) |
| (Acc# X97674) | | | | |
| mRNA for vacuolar proton ATPase, subunit D | 0.83 | 0.80 | 3/11 (27) | 0/10 (0) |
| (Acc# X71490) | | | | |
| Sorcin (SRI) (Acc# L12387) | 0.98 | 0.85 | 3/11 (27) | 0/10 (0) |
| Sorting nexin 2 (SNX2) (Acc# AF043453) | 0.62 | 1.12 | 3/11 (27) | 0/10 (0) |
| Zinc finger protein C2H2-171 (Acc# U38896) | 0.81 | 1.00 | 3/11 (27) | 0/10 (0) |
| Carbonic anhydrase II (Acc# J03037) | 0.92 | 1.28 | 3/11 (27) | 0/10 (0) |
| DNA polymerase epsilon, catalytic polypeptide | 0.71 | 1.12 | 3/11 (27) | 0/10 (0) |
| (Acc# L40386) | | | | |
| Hexabrachion (HXB) (Acc# M55618) | 0.89 | 0.84 | 3/11 (27) | 0/10 (0) |
| mRNA for KIAA0226 gene (Acc# D86979) | 0.72 | 0.88 | 3/11 (27) | 0/10 (0) |
| T-type calcium channel alpha-1 subunit (Acc# | 0.72 | 1.03 | 3/11 (27) | 0/10 (0) |
| AF051946) | | | | |
| CASK (Acc# AF035582) | 0.72 | 0.89 | 3/11 (27) | 0/10 (0) |
| H4 (D10S170), putative cytoskeletal protein (Acc# | 0.72 | 0.88 | 3/11 (27) | 0/10 (0) |
| S72869) | | | | |
| mRNA for inhibitor 2 gene (Acc# X78873) | 0.63 | 0.77 | 3/11 (27) | 0/10 (0) |
| mRNA for RanBP1 (Ran-binding protein 1) | 0.69 | 0.89 | 3/11 (27) | 0/10 (0) |
| (Acc# D38076) | | | | |
| mRNA for transcriptional activator hSNF2b | 0.59 | 0.84 | 3/11 (27) | 0/10 (0) |
| (Acc# D26156) | | | | |
| PDGF associated protein. (Acc# U41745) | 0.75 | 0.91 | 3/11 (27) | 0/10 (0) |
| REC1L mRNA (Acc# X57303) | 0.55 | 0.90 | 3/11 (27) | 0/10 (0) |
| CDC2 gene involved in cell cycle control (Acc# | 0.68 | 1.04 | 3/11 (27) | 0/10 (0) |
| X05360) | | | | |
| Complement component C3 mRNA, alpha and | 0.73 | 1.08 | 3/11 (27) | 0/10 (0) |
| beta subunits (Acc# K02765) | | | | |
| mRNA for C3G protein (Acc# D21239) | 0.91 | 1.01 | 3/11 (27) | 0/10 (0) |
| mRNA for erythrocyte-specific AMP deaminase | 0.81 | 0.89 | 3/11 (27) | 0/10 (0) |
| (Acc# D12775) | | | | |
| mRNA for KIAA0256 gene (Acc# D87445) | 0.79 | 0.98 | 3/11 (27) | 0/10 (0) |
| mRNA for Ran_GTP binding protein 5 (Acc# | 0.69 | 0.87 | 3/11 (27) | 0/10 (0) |
| Y08890) | | | | |
| mRNA for RNA polymerase II 140 kDa subunit | 0.76 | 0.96 | 3/11 (27) | 0/10 (0) |
| (Acc# X63563) | | | | |
| Myo-inositol monophosphatase 2 (Acc# | 0.84 | 1.01 | 3/11 (27) | 0/10 (0) |
| AF014398) | | | | |
| Sodium-hydrogen exchanger 6 (NHE-6) mRNA, | 0.69 | 0.84 | 3/11 (27) | 0/10 (0) |
| nuclear gene encoding mitochondrial protein | | | | |
| (Acc# AF030409) | | | | |
| TRAF family member-associated NF-kB activator | 0.68 | 0.96 | 3/11 (27) | 0/10 (0) |
| TANK (Acc# U63830) | | | | |
| mRNA for intercellular adhesion molecule-1 | 0.66 | 0.98 | 3/11 (27) | 0/10 (0) |
| ICAM-1 (Acc# X06990) | | | | |
| Pulmonary surfactant-associated protein B (SP-B) | 0.75 | 1.41 | 3/11 (27) | 0/10 (0) |
| (Acc# J02761) | | | | |
| h-neuro-d4 protein (Acc# U43843) | 0.79 | 1.08 | 3/11 (27) | 0/10 (0) |
| LIM-homeobox domain protein (hLH-2) (Acc# | 0.61 | 1.30 | 3/11 (27) | 0/10 (0) |
| U11701) | | | | |
| mRNA for maleylacetoacetate isomerase (Acc# | 0.68 | 1.13 | 3/11 (27) | 0/10 (0) |
| AJ001838) | | | | |
| R kappa B mRNA (Acc# X80878) | 0.99 | 1.15 | 3/11 (27) | 0/10 (0) |
| Spermidine aminopropyltransferase (Acc# | 0.76 | 0.94 | 3/11 (27) | 0/10 (0) |
| AD001528) | | | | |
| threonyl-tRNA synthetase (Acc# M63180) | 0.77 | 1.16 | 3/11 (27) | 0/10 (0) |
| Checkpoint suppressor 1 (Acc# U68723) | 1.06 | 0.96 | 3/11 (27) | 0/10 (0) |
| mRNA for Hrs (Acc# D84064) | 1.33 | 0.99 | 4/11 (36) | 1/10 (10) |
| Ferritin heavy chain (Acc# L20941) | 1.15 | 0.88 | 4/11 (36) | 1/10 (10) |
| MG81 (Acc# L08240) | 0.73 | 1.06 | 4/11 (36) | 1/10 (10) |
| NOF1 (Acc# U39400) | 0.72 | 1.01 | 4/11 (36) | 1/10 (10) |
| Thrombin receptor (Acc# M62424) | 0.57 | 0.87 | 4/11 (36) | 1/10 (10) |
| mRNA for brain cholecystokinin receptor (Acc# | 0.80 | 1.28 | 4/11 (36) | 1/10 (10) |
| D13305) | | | | |
| Epidermal growth factor receptor-binding protein | 0.71 | 0.90 | 4/11 (36) | 1/10 (10) |
| GRB2 (EGFRBP-GRB2) mRNA sequence (Acc# | | | | |
| M96995) | | | | |
| HNK-1 sulfotransferase (Acc# AF033827) | 0.68 | 0.76 | 4/11 (36) | 1/10 (10) |
| Hsp27 ERE-TATA-binding protein (HET) (Acc# | 0.96 | 1.05 | 4/11 (36) | 1/10 (10) |
| U72355) | | | | |
| mRNA for golgi antigen gcp372 (Acc# D25542) | 0.67 | 0.95 | 4/11 (36) | 1/10 (10) |
| mRNA for HLA-DR associated protein I (PHAPI) | 1.03 | 1.20 | 4/11 (36) | 1/10 (10) |
| (Acc# X75090) | | | | |
| mRNA for inositol polyphosphate 5-phosphatase | 0.61 | 1.02 | 4/11 (36) | 1/10 (10) |
| (Acc# Y14385) | | | | |
| mRNA for lysosomal sialoglycoprotein (Acc# | 0.81 | 0.75 | 4/11 (36) | 1/10 (10) |
| D12676) | | | | |
| mRNA for tyrosine phosphatase (Acc# Z68092) | 0.61 | 0.72 | 4/11 (36) | 1/10 (10) |
| Requiem (HREQ) (Acc# AF001433) | 0.64 | 0.79 | 4/11 (36) | 1/10 (10) |
| RNA binding protein Etr-3 (Acc# U69546) | 0.93 | 0.87 | 4/11 (36) | 1/10 (10) |
| B61 (Acc# M57730) | 0.83 | 0.85 | 4/11 (36) | 1/10 (10) |
| mRNA for KIAA0205 gene (Acc# D86960) | 0.68 | 1.07 | 4/11 (36) | 1/10 (10) |
| mRNA for raf oncogene (Acc# X03484) | 0.62 | 0.83 | 4/11 (36) | 1/10 (10) |
| mRNA for ULIP phosphoprotein (Acc# Y15014) | 0.60 | 0.99 | 4/11 (36) | 1/10 (10) |
| Pescadillo (Acc# U78310) | 0.66 | 0.95 | 4/11 (36) | 1/10 (10) |
| Splicesomal protein (SAP 61) (Acc# U08815) | 0.52 | 0.73 | 4/11 (36) | 1/10 (10) |
| mRNA for nuclear protein, NP220 (Acc# | 0.87 | 0.90 | 4/11 (36) | 1/10 (10) |
| D83032) | | | | |
| SOX22 protein (SOX22) (Acc# U35612) | 0.72 | 0.92 | 4/11 (36) | 1/10 (10) |
| DAP-1 mRNA (Acc# U77718) | 0.97 | 0.83 | 4/11 (36) | 1/10 (10) |
| Fibroblast muscle-type tropomyosin (Acc# | 0.61 | 0.79 | 4/11 (36) | 1/10 (10) |
| M121)25 | | | | |
| KIAA0410 (Acc# AB007870) | 0.60 | 1.00 | 4/11 (36) | 1/10 (10) |
| mRNA for 26S proteasome subunit p97 (Acc# | 0.83 | 0.80 | 4/11 (36) | 1/10 (10) |
| D78151) | | | | |
| PBX2 mRNA (Acc# X59841) | 0.99 | 0.97 | 4/11 (36) | 1/10 (10) |
| Ser/Arg-related nuclear matrix protein (SRM160) | 0.54 | 0.72 | 4/11 (36) | 1/10 (10) |
| (Acc# AF020760) | | | | |
| Uroporphyrinogen decarboxylase (Acc# M14016) | 0.55 | 0.85 | 4/11 (36) | 1/10 (10) |
| AF-9 (Acc# L13744) | 0.56 | 0.85 | 4/11 (36) | 1/10 (10) |
| Alpha B-crystallin, Rosenthal fiber component | 0.77 | 0.78 | 4/11 (36) | 1/10 (10) |
| (Acc# S45630) | | | | |
| Calcium-activated potassium channel (Acc# | 0.84 | 1.07 | 4/11 (36) | 1/10 (10) |
| U09384) | | | | |
| Neuronal apoptosis inhibitory protein (Acc# | 0.70 | 0.97 | 4/11 (36) | 1/10 (10) |
| U19251) | | | | |
| Sarcomeric mitochondrial creatine kinase (MtCK) | 0.98 | 1.04 | 4/11 (36) | 1/10 (10) |
| gene (Acc# J05401) | | | | |
| DNA repair protein XRCC9 (XRCC9) (Acc# | 0.82 | 1.18 | 4/11 (36) | 1/10 (10) |
| U15932) | | | | |
| mRNA for KIAA0130 gene (Acc# D50919) | 0.72 | 1.02 | 4/11 (36) | 1/10 (10) |
| KIAA0398 (Acc# AB007858) | 0.67 | 0.97 | 4/11 (36) | 1/10 (10) |
| mRNA for dihydrolipoamide succinyltransferase | 0.69 | 0.88 | 4/11 (36) | 1/10 (10) |
| (Acc# D16373) | | | | |
| mRNA for heterogeneous nuclear | 0.69 | 0.95 | 4/11 (36) | 1/10 (10) |
| ribonucleoprotein D (hnRNP D), clone cDx9 | | | | |
| (Acc# D55674) | | | | |
| Myelin basic protein (MBP) (Acc# AF014398) | 0.57 | 1.03 | 4/11 (36) | 1/10 (10) |
| SWI/SNF complex 60 KDa subunit (BAF60b) | 0.66 | 0.93 | 4/11 (36) | 1/10 (10) |
| (Acc# U66618) | | | | |
| Inhibin A-subunit (Acc# M13981) | 0.64 | 0.89 | 4/11 (36) | 1/10 (10) |
| mRNA for putative GTP-binding protein (Acc# | 0.79 | 1.05 | 4/11 (36) | 1/10 (10) |
| Y14391) | | | | |
| mRNA for cytokeratin 13 (Acc# X52426) | 0.70 | 0.98 | 4/11 (36) | 1/10 (10) |
| mRNA for unknown product (Acc# D28124) | 0.55 | 0.94 | 4/11 (36) | 1/10 (10) |
| Gamma-glutmyl transpeptidase-related protein | 0.81 | 2.10 | 4/11 (36) | 1/10 (10) |
| (GGT-Rel) (Acc# M64099) | | | | |
| mRNA for U3 snoRNP associated 55 kDa protein | 0.73 | 1.20 | 5/11 (45) | 2/10 (20) |
| (Acc# AJ001340) | | | | |
| mRNA for 27-kDa calbindin (Acc# X06661) | 0.79 | 0.85 | 5/11 (45) | 2/10 (20) |
| Calnexin (Acc# L18887) | 0.54 | 1.09 | 5/11 (45) | 2/10 (20) |
| mRNA for GARS protein (Acc# D32051) | 0.51 | 0.91 | 5/11 (45) | 2/10 (20) |
| mRNA for PKU-beta (Acc# AS004885) | 0.57 | 0.82 | 5/11 (45) | 2/10 (20) |
| mRNA for protein containing MBD 1 (Acc# | 0.58 | 1.01 | 5/11 (45) | 2/10 (20) |
| Y10746) | | | | |
| Chromosome 17 unknown product (Acc# | 0.57 | 0.87 | 5/11 (45) | 2/10 (20) |
| U58856) | | | | |
| Lysosomal pepstatin insensitive protease (CLN2) | 1.48 | 0.99 | 5/11 (45) | 2/10 (20) |
| (Acc# AF017456) | | | | |
| mRNA for KIAA0273 gene (Acc# D87463) | 0.55 | 0.86 | 5/11 (45) | 2/10 (20) |
| (clone FBK III 16) protein tyrosine kinase (NET | 0.70 | 0.94 | 5/11 (45) | 2/10 (20) |
| PTK) (Acc# L40636) | | | | |
| Grb2-associated binder-1 (Acc# U43885) | 0.59 | 0.94 | 5/11 (45) | 2/10 (20) |
| Mitochondrial HSP75 (Acc# L15189) | 0.51 | 0.85 | 5/11 (45) | 2/10 (20) |
| mRNA for KIAA0258 gene (Acc# D87447) | 0.64 | 1.03 | 5/11 (45) | 2/10 (20) |
| Mevalonate pyrophosphate decarboxylase (MPD) | 0.50 | 0.89 | 5/11 (45) | 2/10 (20) |
| (Acc# U49260) | | | | |
| mRNA for KIAA0275 gene (Acc# D87465) | 0.54 | 0.84 | 5/11 (45) | 2/10 (20) |
| U21.1 mRNA (Acc# X65488) | 0.80 | 1.46 | 5/11 (45) | 2/10 (20) |
| CAGH3 (Acc# U80747) | 0.52 | 0.70 | 5/11 (45) | 2/10 (20) |
| C-terminal binding protein 2 (Acc# X60114) | 0.61 | 0.94 | 5/11 (45) | 2/10 (20) |
| KIAA0408 (Acc# AB007868) | 0.53 | 0.83 | 5/11 (45) | 2/10 (20) |
| mRNA for salivary proline rich peptide P-B | 0.51 | 1.00 | 5/11 (45) | 2/10 (20) |
| (Acc# D29833) | | | | |
| Putative fatty acid desaturase MLD (Acc# | 0.52 | 0.75 | 5/11 (45) | 2/10 (20) |
| AF002668) | | | | |
| mRNA for KIAA0359 gene (Acc# AB002357) | 0.83 | 0.93 | 5/11 (45) | 2/10 (20) |
| Translation initiation factor eIF3 p66 subunit | 0.81 | 0.92 | 5/11 (45) | 2/10 (20) |
| (Acc# U54558) | | | | |
| Ig rearranged kappa-chain mRNA variable region, | 0.81 | 0.99 | 5/11 (45) | 2/10 (20) |
| joining region, constant region (Acc# L03555) | | | | |
| DNA fragmentation factor-45 (Acc# AF011793) | 0.52 | 2.04 | 5/11 (45) | 2/10 (20) |

Additional sequences that were over- and underexpressed in patients with Crohn's disease were identified using significance analysis of the microarray data These additional genes are listed in Table 4.

TABLE 4
Additional Under- and Over- Expressed Sequences
in PBMC's from patients with Crohn's Disease

| Gene Name [GenBank Acc #] (Chromosomal Location) | Expression |
|---|---|
| BAI2. [AB005298] (1p35) | Under |
| Brain MTG8 related protein. [AF0052211] (16q24) | Under |
| Maleylacetoacetate isomerase | Under |
| (glutathione transferase zeta) [AJ001838] (14q24.3) | |
| Regenerating protein 1 beta [D16816] (2p12) | Under |
| Ribosomal protein L4 [D23660] (15q) | Under |
| KIAA0054 (helicase) [D29677] (17q24) | Under |
| Glycyl tRNA synthetase. [D30658] (7p15) | Under |
| KIAA0064 (sorting nexin 17) [D31764] (2p23-22) | Under |
| Carnitine palmitoyltransferase I [D87817] (11q13.1-q13.2) | Under |
| “Dynamin mRNA, alternative exons [L07807] (9q34)” | Under |
| Amyloid protein homologue. [Acc# L09209] (11q24) | Under |
| TR3 orphan receptor (nuclear receptor subfamily 4) [L13740] (12q13) | Under |
| “Immunoglobulin heavy chain, V region. [Acc# L22587] (14q32.33)” | Under |
| RLIP76 protein (Ra1A binding protein 1) [L42542] (18p11.3) | Under |
| Alpha enolase. [Acc# M14328] (1p36.3-p36.2) | Under |
| “2′,3′-cyclic nucleotide 3′-phosphodiesterase. [Acc# M19650] (17q21)” | Under |
| Brain-type clathrin light-chain b [M20469] (4q21-qter) | Under |
| MHC Class I HLA-C-alpha-2 chain. [M24097] (6p21.3) | Under |
| Myosin heavy chain. [Acc# M35250] (17p13.1) | Under |
| Erythrocyte membrane protein [M81635] (unknown) | Under |
| High mobility group box (SSRPI) [M86737] (11q12) | Under |
| Voltage-dependent calcium channel beta-1 subunit [M92302] (17q21-q22) | Under |
| Pre-B cell enhancing factor [U02020] (7q22.1-q31.33) | Under |
| Aspartyl beta-hydroxylase. [U03109] (8q12.1) | Under |
| PITSLRE alpha 1 (cell division cycle 2-like 2) [U04815] (1p36.3) | Under |
| Tissue inhibitor of metalloproteinases-3 [U14394] (22q12.1-q13.2) | Under |
| Pancreatic carboxypeptidase A2 [U19977] (7q32) | Under |
| Dystonin isoform 1 [U31850] (unknown) | Under |
| D53 (hD53) [Acc# U44427] (6) | Under |
| DNA fragmentation factor-45. [Acc# U91985] (1p36.3-p36.2) | Under |
| Amyloid A4 (751) protein. [X06989] (21q21) | Under |
| Slow skeletal troponin C. [X07897] (3p21.3-p14.3) | Under |
| Potential laminin-binding protein (Laminin receptor 1) [X15005] (3p21.3) | Under |
| Liver-type-1-phosphofructokinase. [X15573] (21q22.3) | Under |
| Enteric smooth muscle gamma-actin. [Acc# X16940] | Under |
| Integrin alpha 6 [X53586] (2p14-q14.3) | Under |
| ACTB mRNA for mutant beta-actin (beta$$-actin) [Acc# X63432] | Under |
| (7p15-p12) | |
| Mitochondrial isocitrate dehydrogenase (NADP+) [Acc# X69433] | Under |
| Testican (Spock) [X73608] (5q31) | Under |
| SEX gene [X87852] (Xq27.1-q27.3) | Under |
| Drg1. [X92845] (22q12.2) | Under |
| Immunoglobulin kappa light chain [Y14736] (2p12) | Under |
| KIAA0373. [AB002371] (12) | Over |
| KIAA0433. [AB007893] (5p14.2-q15) | Over |
| Actin-binding double-zinc-finger LIM kinase protein [Acc# AF005654] | Over |
| (10q25) | |
| Chromodomain helicase DNA binding protein 2. [AF006514] (15q26) | Over |
| (clone pGHSCBS) cystathionine beta-synthase. [Acc# L19501] (21q22.3) | Over |
| Acetylcholinesterase (ACHE) [Acc# M55040] (7q22) | Over |
| Endogenous retrovirus type C oncovirus sequence. [M74509] (unknown) | Over |
| Lysosomal alpha-mannosidase (MANB) [U05572] (19cen-q13.1) | Over |
| Beta2-chimaerin. [U07223] (7p15.3) | Over |
| G2 protein [U10991] (11p13) | Over |
| EIF-2-associated p67 protein (Methionyl aminopeptidase 2) [U13261] (12) | Over |
| Nuclear protein Skip (Ski-interacting protein) [U51432] (14q22.1-q24.3) | Over |
| Dishevelled 3. [U75651] (3q27) | Over |
| STM-7 (PIP5K1B) [X92493] (9q13) | Over |
| Carcinoembryonic antigen (CEA) family member 2. [X98311] (19q13.2) | Over |
| Chromogranin B. [Y00064] (20pter-p12) | Over |
| Surface glycoprotein (PTTG1IP) [Z50022] (21q22.3) | Over |

Additional sequences that were over- and underexpressed in patients with ulcerative colitis e were identified using significance analysis of the microarray data. These additional genes are listed in Table 5

TABLE 5
Additional Under- and Over-Expressed Sequences
in PBMC's from patients with Ulcerative Colitis

| Gene Name [GenBank Acc #] (Chromosomal Location) | Direction |
|---|---|
| Skeletal beta-tropomyosin. [X06825] (9p13.2-p13.1) | Over |
| Dihydropyrimidinase related protein 4 [AB006713] (10q26) | Over |
| Ribosomal protein L10 [AB007170] (Xq28) | Over |
| KIAA0407-Plexin B1 [AB007867] (3p24.3-p22.1) | Over |
| Aquaporin 9. [AB008775] (15q22.1-q22.2) | Over |
| Delta [AF003522] (unknown) | Over |
| FGF-1 intracellular binding protein [AF010187] (11q13.1) | Over |
| “i-beta-1,3,-N-acetylglucosaminyltransferase [AF029893] (11)” | Over |
| RGS5 [AF030108] (1q23) | Over |
| Cre binding protein-like 2 [AF039081] (12p13) | Over |
| Protein phosphatase 2C alpha [AF070670] (14q22.1-q22.3) | Over |
| Putative progesterone binding [AJ002030] (4q26) | Over |
| GC Bpx binding protein BTEB2 [D14520] (13q21.2-q22.2) | Over |
| BDR-2 mRNA for hippocalcin [D16593] (1) | Over |
| “Type 3 inositol 1,4,5-triphosphate receptor [D26351] (6pter-p21)” | Over |
| KIAA0115-DDOST [D29643] (1p36.1) | Over |
| KIAA0059-Actin-binding LIM protein 1 [D31883] (10q25) | Over |
| Zinc finger protein [D45213] (unknown) | Over |
| Nucleosome assembly protein 2 [D50370] (Xq21.3-q22) | Over |
| Transcriptional regulator homolog RPD3 [D50405] (1p34.1) | Over |
| TGF-beta IIR alpha [D50683] (3p22) | Over |
| Cardiac calsequestrin [D55655] (1p13.3-p11) | Over |
| Myocilin [D88214] (1q23-q24) | Over |
| Insulinoma rig analog (ribosomal protein S15) [J02984] (19p) | Over |
| Topoisomerase 1 [J03250] (20q12-q13) | Over |
| Calmodulin [J04046] (19q13.2-q13.3) | Over |
| Prolidase [J04605] (19q12-q13.2) | Over |
| Alpha-1-antitrypsin [K01396] (14q32.1} | Over |
| Prealbumin (transthyretin) [K02091] (18q12.1) | Over |
| IEF SSP 9502 [L07758] (unknown) | Over |
| TR3 orphan receptor [L13740] (12q13) | Over |
| Dynamin (DNM) [L36983] (19p) | Over |
| Adhalin mRNA with an R266C mutation [Acc# L46810] (17q12-q21.3) | Over |
| Creatine kinase B [L47647] (14q32) | Over |
| Transcription factor ZFM1 isoform B3 [L49345] (11q13) | Over |
| ERK3 [L77964] | Over |
| Ferritin H chain [M11146] (11q12-q13) | Over |
| H3.3 histone, class B [M11354] (1q41) | Over |
| Creatine kinase M. [M14780] (19) | Over |
| Lipoprotein lipase [M15856] (8p22) | Over |
| Slow skeletal muscle troponin T, clone M [M19308] (19q13.4) | Over |
| Brain-type clathrin light chain b [M20469] (4q2-q3) | Over |
| Protective protein [M22960] (20q13.1) | Over |
| MHC protein homologous to chicken complex B protein [Acc# M24194] | Over |
| Protein kinase related oncogene [M24779] (6p21.2) | Over |
| DNA-repair protein (XRCC1) [M36089] (19q13.2) | Over |
| 47-kD autosomal dominant CGD protein [M55067] (7q11.23) | Over |
| Protein kinase C-L (PRKCL) [M55284] (14q22-q23) | Over |
| Zinc finger protein 42 (MZF-1) [M58297] (19q13.2-q13.4) | Over |
| Growth/differentiation factor 1 (GDF-1) [M62302] (19p12) | Over |
| Protein phosphatase-1 catalytic subunit [M63960] (11q13) | Over |
| ORF [M68864] (unknown) | Over |
| Spermidine/spemine N1-acetyltransferase [M77693] (Xp22.1) | Over |
| I-Rel (RELB) [M83221] (19q13.2) | Over |
| Transcription factor (HTF4A) [M83233] (15q21) | Over |
| Skeletal muscle alpha 2 actinin [M86046] (1q42-q43) | Over |
| PC1(proprotein convertase subtilisin/kexin type 1) [M90753] (5q15-q21) | Over |
| Beta 2-mu, beta 2-microglobulin [S54761] (15q21-q22) | Over |
| 5E10 antigen (Thy-1) [S59749] (11q22.3-23) | Over |
| T3 receptor associating cofactor-1 [S83390] (12q24) | Over |
| Pre-B cell enhancing factor [U02020] (unknown) | Over |
| Protein tyrosine kinase [U02680] (unknown) | Over |
| Actin bundling protein (Drosophila singed homolog) [U03057] (7p22) | Over |
| Chaperonin 10 (GroES) [U07550] (2) | Over |
| hTAK1 [U10990] (3p25) | Over |
| Ribosomal protein L21 [U14967] (13) | Over |
| Ribosomal protein S29 [U14973] (14q) | Over |
| AF1q [U16954] (1q21) | Over |
| bZIP protein NF-IL3A [U26173] (9q22.1-q22.3) | Over |
| Galactokinase [U26401] (17q24) | Over |
| Id-related helix-loop-helix protein Id4 [U28368] (6p22-p21.3) | Over |
| Fragile X mental retardation syndrome related [U31501] (17p13.1) | Over |
| Vacuolar protein sorting homolog h-vps45 [U35246] (1) | Over |
| Ribosomal protein L23a [U37230] (unknown) | Over |
| Protein phosphatase 2A B$$alpha1 [U37352] (3p21) | Over |
| Myosin VIIa transcript 2 [U39226] (11q13.5) | Over |
| Patched homolog (PTC) [U43148] (9q22.3) | Over |
| Putative cerbral cortex transcriptional regulator [U49250] (2q23-q27) | Over |
| Lysosomal-associated multitransmembrane protein [U51240] (1p34) | Over |
| Armadillo repeat protein [U51269] (Xq21.33-q22.2) | Over |
| Desmin [U59167] (2q35) | Over |
| Leptin receptor splice variant [U66496] (1p31) | Over |
| Ninjurin 1 [U72661] (9q22) | Over |
| MOP4 (NPAS2) [U77970] (2p11.2-q13) | Over |
| Immunophilin homolog ARA9 [U78521] (11q13.3) | Over |
| Frizzled homolog (FZD3) [U82169] (7q11.23) | Over |
| Nucleolar protein p40 [U86602] (1p35-p33) | Over |
| Steroid receptor coactivator-1 [U90661] (2p23) | Over |
| Erythrocyte 2,3-biphosphoglycerate mutase [X04327] (7q31-q34) | Over |
| Lymphocyte activation marker Blast-1 [X05341] (1q21.3-q22) | Over |
| Lipocortin (Annexin A1) [X05908] (9q11-q22) | Over |
| Slow skeletal troponin C [X07897] (3p21.3-p14.3) | Over |
| Ribosomal protein L31 [X15940] (2q) | Over |
| COX VIIc [X16560] (5q14) | Over |
| L23 mRNA for putative ribosomal protein [X53777] (18q) | Over |
| Neural cell adhesion molecule [X55322] (11q23.1) | Over |
| TFIIS for transcription elongation factor [X57198] (3p22-p21.3) | Over |
| Cyl mRNA for cytoplasmic kinase (CSK) [X60114] (15q23-q25) | Over |
| ERK1 [X60188] (16p11.2) | Over |
| Dek [X64229] (6p23) | Over |
| M6 antigen [X64364] (19p13.3) | Over |
| HPBRII-4 [X67337] (12) | Over |
| ORF (Ribosomal protein L18a) [X80822] (19p) | Over |
| Cytokine inducible nuclear protein [X83703] (10) | Over |
| BiP protein (Heat shock 70 kD protein 5) [X87949] (9q34) | Over |
| DNA binding protein A variant [X95325] (12p13.1) | Over |
| E14 protein [X97186] (11q22.3) | Over |
| TYL gene [X99688] (10q24) | Over |
| Polypeptide 7B2 [Y00757] (15q11-q15) | Over |
| Manganese-containing SOD [Y00985] (6p25.3) | Over |
| Caveolin [Z18951] (7q31.1) | Over |
| HnRNP G protein [Z23064] (Xq26) | Over |
| Ribosomal protein L38 [Z26876] (17q) | Over |
| CLPP [Z50853] (19p13.3) | Over |
| KIAA0331-Semaphorin 3E [AB002329] (7q21.11-q31.33) | Under |
| RES4: 26 [AB00468] (4p16.3) | Under |
| Ubiquitin-conjugating enzyme UbcH7 [AJ000519] (22q11.2-q13.1) | Under |
| Doublecortin [AJ003112] (Xq22.3-q23) | Under |
| HBp15/L22 [D17652] (3q26) | Under |
| KIAA0232 [D86985] (4) | Under |
| Cytochrome bc-1 complex core protein II [J04973] (16) | Under |
| Dematin (HRD1) [L19713] (8p21.1) | Under |
| Aldolase A [M11560] (16q22-q24) | Under |
| Mitochondrial aspartate aminotransferase [M22632] (16q21) | Under |
| Myosin light chain 3, non-muscle [M31212] (12) | Under |
| Tumor necrosis factor receptor [M33294] (12p13.2) | Under |
| Myosin heavy chain (MYH8) [M35250] (17p13.1) | Under |
| Keratin, keratin 16 homolog [S72493] (17q12-q21) | Under |
| Epidermal growth factor receptor substrate (eps15) [U07707] (1p32) | Under |
| ZNF174 [U31248] (16p13.3) | Under |
| Spliceosome associated protein (SAP145) [U41371] (11) | Under |
| Dishevelled homolog [U46461] (1p36) | Under |
| p167 [U58046] (10q26) | Under |
| Putative cyclin G1 interacting protein [U61837] (7) | Under |
| YF5 [U84569] (21q22.3) | Under |
| Rap1A [X12533] (1p13.3) | Under |
| RECIL [X12533] 1p13.3) | Under |
| Sm protein F [X85372] (4q31.1-q31.23) | Under |
| Coronin [X89109] (16p12.2-q12.2) | Under |
| TFG [Y07968] (3q11-q12) | Under |
| Immunoglobulin lambda heavy chain [Y14737] (unknown) | Under |
| Myosin [Z38133] (17p13.1) | Under |

### EXAMPLE 4

### Diagnostic Array for Patients with IBD, Crohn's Disease and Ulcerative Colitis

Using the microarray data generated in Example 3, cluster analysis was performed to identify clusters of related genes that may be involved in disease processes using the Cluster Analysis software kindly provided, by Michael Eisen on the internet (http://rana.stanford.edu/software) See M. B. Eisen et al., “Cluster analysis and display of genome-wise expression patterns,” Proc. Natl. Acad. Sci. USA, vol. 95, pp. 14863-14868 (1998)

Cluster analysis indicated that by using 24 genes, IBD could be identified in our patient sample with a sensitivity of 84% and a specificity of 100%. By using 25 genes (the 24 genes plus one additional gene), IBD could be identified with a sensitivity of 88% and a specificity of 90%. The genes identified are the following: Acidic calponin (Acc# S80562), Beta-sarcoglycan A3b (Acc# U31116), CL100 mRNA for protein tyrosine phosphatase (Acc# X68277), ZIP-kinase (Acc# AB007144), G protein gamma-4 subunit (Acc# U31382), Fibroblast muscle-type tropomyosin (Acc# M12125), Alkali myosin light chain 1 (Acc# M20642), Achaete scute homologous protein (Acc# L08424), Sorting nexin 2 (SNX2) (Acc# AF043453), Tristetraproline (TTP) (Acc# M63625), serine/threonine protein kinase (#D86550) (Acc# U59305), KIAA0210 (Acc# D86965), Methionine aminopeptidase (Acc# U29607), (p23) (Acc# L24804), Placenta (Diff48) (Acc# U49187), Rac3 (RAC3) (Acc# AF008591), Jun-B (Acc# X51345), Tyrosine phosphatase (Acc# Z68092), Complement component C3 mRNA, alpha and beta subunits (Acc# K02765), PKC alpha (Acc# X52479), SHPS-1 (Acc# D86043), Hbrm (Acc# X72889), Maleylacetoacetate isomerase(Acc# AJ001838), mRNA encoding GPI-anchored protein p137 (Acc# Z48042). The second cluster is composed of these 24 genes plus one additional gene, Homolog of Drosophila enhancer of split m9/m10(Acc# U04241).

Additionally, cluster analysis indicated that by using thirty-six genes, patients with Crohn's disease could be distinguished from patients with ulcerative colitis with a sensitivity of 89% and a specificity of 80% for Crohn's disease, and a sensitivity of 89% and specificity of 80% for ulcerative colitis. These thirty-six genes were identified as the following: ( )) (Acc# L24804), (Acc# D21267); 14-3-3 protein epsilon isoform (Acc# U43399); 47 kD autosomal chronic granulomatous disease protein (Acc# M55067); Adaptor protein XII beta (Acc# AF047348); Adenylate kinase 2 (adk2) (Acc# U39945); Adenylyl cyclase-associated protein (CAP) (Acc# L12168); alpha-CP1 (Acc# U24223); Apoptotic protease activating factor 1 (Apaf-1) (Acc# AF013263); cAMP-dependent protein icinase subunit RII-beta (Acc# M31158); COX VIIc gene for subunit VIIc of cytochrome c oxidase (EC 1.9.3.1) (Acc# U53328); Arp2/3 protein complex subunit p34-Arc (ARC34) (Acc# AF006085); D53 (hD53) (Acc# M77830); Desmin (Acc# AF006012); Dynamin (DNM) (Acc# S72422); H5; and platelet glycoprotein Ib beta chain (Acc# U59632); Keratin, keratin 16 homolog (Acc# S72493); Lymph node homing receptor (Acc# M25280); Lysosomal-associated multitransmembrane protein (LAPTm5) (Acc# U51240); MHC protein homologous to chicken B complex protein (Acc# M24194); mRNA for Clock (Acc# AB005535); mRNA for lymphocte activation marker Blast-1 (Acc# X06341); mRNA for ORF (Acc# X80822); mRNA for ZIP-kinase (Acc# AB007144); Myosin alkali light chain (ventricular) (Acc# M24122); Myosin light chain 3 non-muscle MLC3 nm) (Acc# M31212); N-acetylglucosaminyltransferase I (GlcNAc-TI) (Acc# M55621); Nicotinic acetylcholine receptor alpha3 subunit precursor, (Acc# U62432); p167 (Acc# U58046); P2X1 receptor (Acc# U45448); Partial C1 mRNA (Acc# X78817); Protein-tyrosine kinase (JAK1) (Acc# M64174); rab2 mRNA, YPT1-related and member of ras family (Acc# X12953); Retinoid X receptor beta (RXR-beta) (Acc# M84820); Sorbitol dehydrogenase gene (Acc# U07361); Wilm's tumor-related protein (QM) (Acc# M64241).

Using the above information, a microarray could be custom-made using cDNAs of the above sequences, along with housekeeping sequences and plant or other control sequences. Such a custom-made microarray with cDNA from fewer sequences than the original 2400 sequences would decrease the cost of using a microarray as a diagnostic tool for IBD. The microarray could include all genes identified for IBD, Crohn's, and UC as listed in Table 2. The better method would be a microarray that would contain the twenty-five sequences that can identify IBD, and the thirty-six sequences that could then distinguish Crohn's from ulcerative colitis. Such a microarray would contain the following sixty genes (because one sequence overlaps in the two lists): Acidic calponin (Acc# S80562), Beta-sarcoglycan A3b (Acc# U31116), CL100 mRNA for protein tyrosine phosphatase (Acc# X68277), ZIP-kinase (Acc# AB007144), G protein gamma-4 subunit (Acc# U31382), Fibroblast muscle-type tropomyosin (Acc# M12125), Alkali myosin light chain 1 (Acc# M20642), Achaete scute homologous protein (Acc# L08424), Sorting nexin 2 (SNX2) (Acc# AF043453), Tristetraproline (TTP) (Acc# M63625), serine/threonine protein kinase (#D86550) (Acc# U59305), KIAA0210 (Acc# D86965), Methionine aminopeptidase (Acc# U29607), (p23) (Acc# L24804), Placenta (Diff48) (Acc# U49187), Rac3 (RAC3) (Acc# AF008591), jun-B (Acc# X51345), tyrosine phosphatase (Acc# Z68092), Complement component C3 mRNA, alpha and beta subunits (Acc# K02765), PKC alpha (Acc# X52479), SHPS-1 (Acc# D86043), Hbrm (Acc# X72889), maleylacetoacetate isomerase(Acc# AJ001838), mRNA encoding GPI-anchored protein p137 (Acc# Z48042), Homolog of Drosophila enhancer of split m9/m10 (Acc# U04241), (p23) (Acc# L24804), (Acc# D21267), 14-3-3 protein epsilon isoform (Acc# U43399); 47 kD autosomal chronic granulomatous disease protein (Acc# M55067); Adaptor protein XII beta (Acc# AF047348); Adenylate kinase 2 (adk2) (Acc# U39945); Adenylyl cyclase-associated protein (CAP) (Acc# L12168); alpha-CP1 (Acc# U24223); Apoptotic protease activating factor 1 (Apaf-1) (Acc# AF013263); cAMP-dependent protein kinase subunit RII-beta (Acc# M31158); COX VIIc gene for subunit VIIc of cytochrome c oxidase (EC 1.9.3.1); (Acc# U53328); Arp2/3 protein complex subunit p34-Arc (ARC34) (Acc# AF006085); D53 (hD53) (Acc# M77830); Desmin (Acc# AF006012); Dynamin (DNM) (Acc# S72422); H5; and platelet glycoprotein Ib beta chain (Acc# U59632); Keratin, keratin 16 homolog (Acc# S72493); Lymph node homing receptor (Acc# M25280); Lysosomal-associated multitransmembrane protein (LAPTm5) (Acc# U51240); MHC protein homologous to chicken B complex protein (Acc# M24194); mRNA for Clock (Acc# AB005535); mRNA for lymphocte activation marker Blast-1 (Acc# X06341); mRNA for ORF (Acc# X80822); Myosin alkali light chain (ventricular) (Acc# M24122); Myosin light chain 3 non-muscle (MLC3 nm) (Acc# M31212); N-acetylglucosaminyltransferase I (GlcNAc-TI) (Acc# M55621); Nicotinic acetylcholine receptor alpha3 subunit precursor, (Acc# U62432); p167 (Acc# U58046); P2x1 receptor (Acc# U45448); Partial C1 mRNA (Acc# X78817); Protein-tyrosine kinase (JAK1) (Acc# M64174); rab2 mRNA, YPT1-related and member of ras family (Acc# X12953); Retinoid X receptor beta (RXR-beta) (Acc# M84820); Sorbitol dehydrogenase gene (Acc# U07361); Wilm's tumor-related protein (QM) (Acc# M64241).

The following additional genes were identified as significant diagnostic genes for inflammatory bowel disease, Crohn's Disease, and ulcerative colitis: 2′,3′-cyclic nucleotide3′-phosphodiesterase (Acc# M19650), D53(hD53) (Acc# U44427), DNA fragmentation factor-45 (Acc# U91985), Drg1 (Acc# X92845), Epidermal growth factor receptor substrate (eps15) (Acc# U07707),Myosin heavy chain (Acc# M35230), ARL1 (Acc# L28997), Clone 22 mRNA, alternative splice variant beta-2 (AF009424), Glutathione-S-transferase homolog (Acc# U90313), Leukemia virus receptor 2 (Acc# L20852), Muscle specific enolase (Acc# X51957), Type 3 inositol 1,4,5-triphosphate receptor (D26351), Hrs (Acc# D84064).

### EXAMPLE 5

### Confirmation of Gene Expression

Studies to confirm the expression of 10 disease-related genes identified above will be performed. Preliminary analysis or 7 disease-related genes have been confirmed. These studies used SYBR green real-time PCR on RNA stored from the initial PBMC extraction. SYBR green real-time PCR involves the introduction of a SYBR green dye that fluoresces when bound to double-stranded (ds) DNA. During amplification, fluorescent measurements were taken and a threshold cycle (Cₜ) value for each sample was calculated to determine the cycle time at which the fluorescent intensity exceeded a threshold (10 times the standard deviation from baseline emissions). Threshold cycle, or sample positivity, was directly proportional to the amount of target material allowing quantization of gene expression. Primers designed for the genes of interest was designed and tested for primer stability using ABI Primer Express software (Applied Biosystems, Foster City, Calif.) and synthesized by Integrated DNA technologies (Coral Ville, Iowa). Briefly, reverse transcription of 100 ng of RNA was performed using Rnase H-deficient reverse transcriptase (Superscript II, Life Technologies, Rockville, Md.) and Random Hexamer primers. The PCR reaction was performed using the Taq-Man Core PCR kit (PE Applied Biosystems) in 96-well trays with optical caps containing triplicates of each sample. The reaction mixture was generally preheated for 10 minutes at 95° C., then 35 cycles of 15 seconds at 95° C. and one minute at 60° C., followed by one cycle of heating from 60° C. to 95° C. over 20 minutes to obtain a melting curve of the PCR products and carried out by a 7700 Sequence Detector (PE Applied Biosystems). PCR conditions for each of the overexpressed and underexpressed genes was optimized including primer concentration and magnesium concentration. The relative gene expression is determined based on the normalized threshold cycle of the gene of interest to a pool of unaffected control samples to control for transcription efficiency. The normalized Cₜ value obtained for each gene for IBD samples was compared with the value obtained for each gene for unaffected control samples. Ratios of relative gene expression are then compared with microarray results. Preliminary results confirmed relative expression for the following seven genes: Acidic Calponin (Accession #S80562), Arp 2/3 protein complex subunit 34-Arc (ARC34) (Accession #AF006085), Human antigen CD36 (clone 13) mRNA (Accession #M98398), Human epidermal growth factor receptor substrate (eps15) (Accession #U07707), mRNA for Hrs (Accession #D84064), Serine threonine kinase 11 (STKL 11) (Accession #AF035625), and mRNA for SHPS-1 (Acc# D86043).

The complete disclosures of all references cited in this specification are hereby incorporated by reference. Also incorporated by reference is the full disclosure of the following poster: M.-S. Serrano et al., “Application of microarray analysis to Crohn's disease,” World Congress of Pediatric Gastroenterology and Nutrition, Boston, Massachusetts, Aug. 7, 2000. In the event of an otherwise irreconcilable conflict, however, the present specification shall control.

What is claimed:

1. A method to diagnose inflammatory bowel disease in a human patient, said method comprising the steps of:
(a) obtaining a sample of body fluid or tissue from the patient;
(b) detecting in said sample a pattern of gene expression of genes selected from the group consisting of Acidic calponin (Acc# S80562), Beta-sarcoglycan A3b (Acc# U31116), CL100 mRNA for protein tyrosine phosphatase (Acc# X68277), ZIP-kinase (Acc# AB007144), G protein gamma-4 subunit (Acc# U31382), Fibroblast muscle-type tropomyosin (Acc# M12125), Alkali myosin light chain 1 (Acc# M20642), Achaete scute homologous protein (Acc# L08424), Sorting nexin 2 (SNX2) (Acc# AF043453), Tristetraproline (TTP) (Acc# M63625), serine/threonine protein kinase (#D86550) (Acc# U59305), KIAA0210 (Acc# D86965), Methionine aminopeptidase (Acc# U29607), (p23) (Acc# L24804), Placenta (Diff48) (Acc# U49187), Rac3 (RAC3) (Acc# AF008591), Jun-B (Acc# X51345), Tyrosine phosphatase (Acc# Z68092), Complement component C3 mRNA, alpha and beta subunits (Acc# K02765), PKC alpha (Acc# X52479), SHPS-1 (Acc# D86043), Hbrm (Acc# X72889), Maleylacetoacetate isomerase(Acc# AJ001838), mRNA encoding GPI-anchored protein p137 (Acc# Z48042) and Homolog of Drosophila enhancer of split m9/m10(Acc# U04241), 2′,3′-cyclic nucleotide3′-phosphodiesterase (Acc# M19650), D53(hD53) (Acc# U44427), DNA fragmentation factor-45 (Acc# U91985), Drg1 (Acc# X92845), Epidermal growth factor receptor substrate (eps15) (Acc# U07707),Myosin heavy chain (Acc# M35230), ARL1 (Acc# L28997), Clone 22 mRNA, alternative splice variant beta-2 (AF009424), Glutathione-S-transferase homolog (Acc# U90313), Leukemia virus receptor 2 (Acc# L20852), Muscle specific enolase (Acc# X51957), Type 3 inositol 1,4,5-triphosphate receptor (D26351), Hrs (Acc# D84064); and
(c) comparing the pattern of gene expression from said sample and one or more entries from a library of known patterns of gene expression from patients with inflammatory bowel disease.

2. The method of claim 1, wherein the sample comprises blood cells from the patient.

3. The method of claim 1, wherein the gene expression is detected using DNA microarray hybridization.

4. The method of claim 3, wherein an oligonucleotide is isolated from the sample.

5. A method to distinguish between the presence of Crohn's disease from the presence of ulcerative colitis in a human patient with inflammatory bowel disease, said method comprising the steps of:
(a) obtaining a sample of body fluid or tissue from the patient;
(b) detecting a pattern of gene expression present in said sample of genes selected from the group consisting of (p23) (Acc# L24804), (Acc# D21267); 14-3-3 protein epsilon isoform (Acc# U43399); 47 kD autosomal chronic granulomatous disease protein (Acc# M55067); Adaptor protein XII beta (Acc# AF047348); Adenylate kinase 2 (adk2) (Acc# U39945); Adenylyl cyclase-associated protein (CAP) (Acc# L12168); alpha-CP1 (Acc# U24223); Apoptotic protease activating factor 1 (Apaf-1) (Acc# AF013263); cAMP-dependent protein kinase subunit RII-beta (Acc# M31158); COX VIIc gene for subunit VIIc of cytochrome c oxidase (EC 1.9.3.1) (Acc# U53328); Arp2/3 protein complex subunit p34-Arc (ARC34) (Acc# AF006085); D53 (hD53) (Acc# M77830); Desmin (Acc# AF006012); Dynamin (DNM) (Acc# S72422); H5; and platelet glycoprotein Ib beta chain (Acc# U59632); Keratin, keratin 16 homolog (Acc# S72493); Lymph node homing receptor (Acc# M25280); Lysosomal-associated multitransmembrane protein (LAPTm5) (Acc# U51240); MHC protein homologous to chicken B complex protein (Acc# M24194); mRNA for Clock (Acc# AB005535); mRNA for lymphocte activation marker Blast-1 (Acc# X06341); mRNA for ORF (Acc# X80822); mRNA for ZIP-kinase (Acc# AB007144); Myosin alkali light chain (ventricular) (Acc# M24122); Myosin light chain 3 non-muscle (MLC3nm) (Acc# M31212); N-acetylglucosaminyltransferase I (GlcNAc-TI) (Acc# M55621); Nicotinic acetylcholine receptor alpha3 subunit precursor, (Acc# U62432); p167 (Acc# U58046); P2x1 receptor (Acc# U45448); Partial C1 mRNA (Acc# X78817); Protein-tyrosine kinase (JAK1) (Acc# M64174); rab2 mRNA, YPT1-related and member of ras family (Acc# X12953); Retinoid X receptor beta (RXR-beta) (Acc# M84820); Sorbitol dehydrogenase gene (Acc# U07361); Wilm's tumor-related protein (QM) (Acc# M64241), 2′,3′-cyclic nucleotide3′-phosphodiesterase (Acc# M19650), D53(hD53) (Acc# U44427), DNA fragmentation factor-45 (Acc# U91985), Drg1 (Acc# X92845), Epidermal growth factor receptor substrate (eps15) (Acc# U07707),Myosin heavy chain (Acc# M35230), ARL1 (Acc# L28997), Clone 22 mRNA, alternative splice variant beta-2 (AF009424), Glutathione-S-transferase homolog (Acc# U90313), Leukemia virus receptor 2 (Acc# L20852), Muscle specific enolase (Acc# X51957), Type 3 inositol 1,4,5-triphosphate receptor (D26351), Hrs (Acc# D84064); and
(c) comparing the pattern of gene expression from said sample with a library of known patterns of gene expression for patients with Crohn's disease and patients with ulcerative colitis.

6. The method of claim 5, wherein the sample comprises blood cells from the patient.

7. The method of claim 5, wherein the gene expression is detected using DNA microarray hybridization.

8. The method of claim 7, wherein a oligonucleotide is isolated from the sample.

9. A method to diagnose inflammatory bowel disease in a human patient, and to distinguish Crohn's disease from ulcerative colitis, said method comprising the steps of:
(a) obtaining a sample of body fluid or tissue from the patient;
(b) detecting a pattern of gene expression present in said sample of genes selected from the group consisting of Acidic calponin (Acc# S80562), Beta-sarcoglycan A3b (Acc# U31116), CL100 mRNA for protein tyrosine phosphatase (Acc# X68277), ZIP-kinase (Acc# AB007144), G protein gamma-4 subunit (Acc# U31382), Fibroblast muscle-type tropomyosin (Acc# M12125), Alkali myosin light chain 1 (Acc# M20642), Achaete scute homologous protein (Acc# L08424), Sorting nexin 2 (SNX2) (Acc# AF043453), Tristetraproline (TTP) (Acc# M63625), serine/threonine protein kinase (#D86550) (Acc# U59305), KIAA0210 (Acc# D86965), Methionine aminopeptidase (Acc# U29607), (p23) (Acc# L24804), Placenta (Diff48) (Acc# U49187), Rac3 (RAC3) (Acc# AF008591), jun-B (Acc# X51345), tyrosine phosphatase (Acc# Z68092), Complement component C3 mRNA, alpha and beta subunits (Acc# K02765), PKC alpha (Acc# X52479), SHPS-1 (Acc# D86043), Hbrm (Acc# X72889), maleylacetoacetate isomerase(Acc# AJ001838), mRNA encoding GPI-anchored protein p137 (Acc# Z48042), Homolog of Drosophila enhancer of split m9/m10 (Acc# U04241), (p23) (Acc# L24804), (Acc# D21267), 14-3-3 protein epsilon isoform (Acc# U43399); 47 kD autosomal chronic granulomatous disease protein (Acc# M55067); Adaptor protein XII beta (Acc# AF047348); Adenylate kinase 2 (adk2) (Acc# U39945); Adenylyl cyclase-associated protein (CAP) (Acc# L12168); alpha-CP1 (Acc# UJ24223); Apoptotic protease activating factor 1 (Apaf-1) (Acc# AF013263); cAMP-dependent protein kinase subunit RII-beta (Acc# M31158); COX VIIc gene for subunit VIIc of cytochrome c oxidase (EC 1.9.3.1); (Acc# U53328); Arp2/3 protein complex subunit p34-Arc (ARC34) (Acc# AF006085); D53 (hD53) (Acc# M77830); Desmin (Acc# AF006012); Dynamin (DNM) (Acc# S72422); H5; and platelet glycoprotein Ib beta chain (Acc# U59632); Keratin, keratin 16 homolog (Acc# S72493); Lymph node homing receptor (Acc# M25280); Lysosomal-associated multitransmembrane protein (LAPTm5) (Acc# U51240); MHC protein homologous to chicken B complex protein (Acc# M24194); mRNA for Clock (Acc# AB005535); mRNA for lymphocte activation marker Blast-1 (Acc# X06341); mRNA for ORF (Acc# X80822); Myosin alkali light chain (ventricular) (Acc# M24122); Myosin light chain 3 non-muscle (MLC3 nm) (Acc# M31212); N-acetylglucosaminyltransferase I (GlcNAc-TI) (Acc# M55621); Nicotinic acetylcholine receptor alpha3 subunit precursor, (Acc# U62432); p167 (Acc# U58046); P2x1 receptor (Acc# U45448); Partial C1 mRNA (Acc# X78817); Protein-tyrosine kinase (JAK1) (Acc# M64174); rab2 mRNA, YPT1-related and member of ras family (Acc# X12953); Retinoid X receptor beta (RXR-beta) (Acc# M84820); Sorbitol dehydrogenase gene (Acc# U07361); Wilm's tumor-related protein (QM) (Acc# M64241), 2′,3′-cyclic nucleotide3′-phosphodiesterase (Acc# M19650), D53(hD53) (Acc# U44427), DNA fragmentation factor-45 (Acc# U91985), Drg1 (Acc# X92845), Epidermal growth factor receptor substrate (eps15) (Acc# U07707),Myosin heavy chain (Acc# M35230), ARL1 (Acc# L28997), Clone 22 mRNA, alternative splice variant beta-2 (AF009424), Glutathione-S-transferase homolog (Acc# U90313), Leukemia virus receptor 2 (Acc# L20852), Muscle specific enolase (Acc# X51957), Type 3 inositol 1,4,5-triphosphate receptor (D26351), Hrs (Acc# D84064); and
(c) comparing the pattern of gene expression of the following genes with a library of known patterns of gene expression from patients with inflammatory bowel disease: Acidic calponin (Acc# S80562), Beta-sarcoglycan A3b (Acc# U31116), CL100 mRNA for protein tyrosine phosphatase (Acc# X68277), ZIP-kinase (Acc# AB007144), G protein gamma-4 subunit (Acc# U31382), Fibroblast muscle-type tropomyosin (Acc# M12125), Alkali myosin light chain 1 (Acc# M20642), Achaete scute homologous protein (Acc# L08424), Sorting nexin 2 (SNX2) (Acc# AF043453), Tristetraproline (TTP) (Acc# M63625), serine/threonine protein kinase (#D86550) (Acc# U59305), KIAA0210 (Acc# D86965), Methionine aminopeptidase (Acc# U29607), (p23) (Acc# L24804), Placenta (Diff48) (Acc# U49187), Rac3 (RAC3) (Acc# AF008591), Jun-B (Acc# X5i345), Tyrosine phosphatase (Acc# Z68092), Complement component C3 mRNA, alpha and beta subunits (Acc# K02765), PKC alpha (Acc# X52479), SHPS-1 (Acc# D86043), Hbrm (Acc# X72889), Maleylacetoacetate isomerase(Acc# AJ001838), mRNA encoding GPI-anchored protein p137 (Acc# Z48042) and Homolog of Drosophila enhancer of split m9/m10(Acc# U04241), 2′,3′-cyclic nucleotide3′-phosphodiesterase (Acc# M19650), D53(hD53) (Acc# U44427), DNA fragmentation factor-45 (Acc# U91985), Drg1 (Acc# X92845), Epidermal growth factor receptor substrate (eps15) (Acc# U07707),Myosin heavy chain (Acc# M35230), ARL1 (Acc# L28997), Clone 22 mRNA, alternative splice variant beta-2 (AF009424), Glutathione-S-transferase homolog (Acc# U90313), Leukemia virus receptor 2 (Acc# L20852), Muscle specific enolase (Acc# X51957), Type 3 inositol 1,4,5-triphosphate receptor (D26351), Hrs (Acc# D84064); and
(d) comparing the pattern of gene expression of the following genes with a library of known patterns of gene expression from patients with Crohn's disease and from patients with ulcerative colitis: (p23) (Acc# L24804), (Acc# D21267); 14-3-3 protein epsilon isoform (Acc# U43399); 47 kD autosomal chronic granulomatous disease protein (Acc# M55067); Adaptor protein XII beta (Acc# AF047348); Adenylate kinase 2 (adk2) (Acc# U39945); Adenylyl cyclase-associated protein (CAP) (Acc# L12168); alpha-CP1 (Acc# U24223); Apoptotic protease activating factor 1 (Apaf-1) (Acc# AF013263); cAMP-dependent protein kinase subunit RII-beta (Acc# M31158); COX VIIc gene for subunit VIIc of cytochrome c oxidase (EC 1.9.3.1) (Acc# U53328); Arp2/3 protein complex subunit p34-Arc (ARC34) (Acc# AF006085); D53 (hD53) (Acc# M77830); Desmin (Acc# AF006012); Dynamin (DNM) (Acc# S72422); H5; and platelet glycoprotein Ib beta chain (Acc# U59632); Keratin, keratin 16 homolog (Acc# S72493); Lymph node homing receptor (Acc# M25280); Lysosomal-associated multitransmembrane protein (LAPTm5) (Acc# U51240); MHC protein homologous to chicken B complex protein (Acc# M24194); mRNA for Clock (Acc# AB005535); mRNA for lymphocte activation marker Blast-1 (Acc# X06341); mRNA for ORF (Acc# X80822); mRNA for ZIP-kinase (Acc# AB007144); Myosin alkali light chain (ventricular) (Acc# M24122); Myosin light chain 3 non-muscle (MLC3 nm) (Acc# M31212); N-acetylglucosaminyltransferase I (GlcNAc-TI) (Acc# M55621); Nicotinic acetylcholine receptor alpha3 subunit precursor, (Acc# U62432); p167 (Acc# U58046); P2x1 receptor (Acc# U45448); Partial C1 mRNA (Acc# X78817); Protein-tyrosine kinase (JAK1) (Acc# M64174); rab2 mRNA, YPT1-related and member of ras family (Acc# X12953); Retinoid X receptor beta (RXR-beta) (Acc# M84820); Sorbitol dehydrogenase gene (Acc# U07361); Wilm's tumor-related protein (QM) (Acc# M64241), 2′,3′-cyclic nucleotide3′-phosphodiesterase (Acc# M19650), D53(hD53) (Acc# U44427), DNA fragmentation factor-45 (Acc# U91985), Drg1 (Acc# X92845), Epidermal growth factor receptor substrate (eps15) (Acc# U07707),Myosin heavy chain (Acc# M35230), ARL1 (Acc# L28997), Clone 22 mRNA, alternative splice variant beta-2 (AF009424), Glutathione-S-transferase homolog (Acc# U90313), Leukemia virus receptor 2 (Acc# L20852), Muscle specific enolase (Acc# X51957), Type 3 inositol 1,4,5-triphosphate receptor (D26351), Hrs (Acc# D84064).

10. The method of claim 9, wherein the sample comprises blood cells from the patient.

11. The method of claim 9, wherein the gene expression is detected using DNA microarray hybridization.

12. The method of claim 11, wherein a oligonucleotide is isolated from the sample.

13. A drug screening assay comprising:
(a) administering a test compound to a mammal having an inflammatory bowel disease, including Crohn's disease or ulcerative colitis, or to intestinal cells isolated from a mammal with inflammatory bowel disease, including Crohn's disease or ulcerative colitis;
(b) obtaining a sample from the mammal or the cell composition;
(c) detecting a pattern of gene expression present in said sample of genes selected from the group consisting of Acidic calponin (Acc# S80562), Beta-sarcoglycan A3b (Acc# U31116), CL100 mRNA for protein tyrosine phosphatase (Acc# X68277), ZIP-kinase (Acc# AB007144), G protein gamma-4 subunit (Acc# U31382), Fibroblast muscle-type tropomyosin (Acc# M12125), Alkali myosin light chain 1 (Acc# M20642), Achaete scute homologous protein (Acc# L08424), Sorting nexin 2 (SNX2) (Acc# AF043453), Tristetraproline (TTP) (Acc# M63625), serine/threonine protein linase (#D86550) (Acc# U59305), KIAA0210 (Acc# D86965), Methionine aminopeptidase (Acc# U29607), (p23) (Acc# L24804), Placenta (Diff48) (Acc# U49187), Rac3 (RAC3) (Acc# AF008591), jun-B (Acc# X51345), tyrosine phosphatase (Acc# Z68092), Complement component C3 mRNA, alpha and beta subunits (Acc# K02765), PKC alpha (Acc# X52479), SHPS-1 (Acc# D86043), Hbrm (Acc# X72889), maleylacetoacetate isomerase(Acc# AJ001838), mRNA encoding GPI-anchored protein p137 (Acc# Z48042), Homolog of Drosophila enhancer of split m9/m10 (Acc# U04241), (p23) (Acc# L24804), (Acc# D21267), 14-3-3 protein epsilon isoform (Acc# U43399); 47 kD autosomal chronic granulomatous disease protein (Acc# M55067); Adaptor protein XII beta (Acc# AF047348); Adenylate kinase 2 (adk2) (Acc# U39945); Adenylyl cyclase-associated protein (CAP) (Acc# L12168); alpha-CP1 (Acc# U24223); Apoptotic protease activating factor 1 (Apaf-1) (Acc# AF013263); cAMP-dependent protein kinase subunit RII-beta (Acc# M31158); COX VIIc gene for subunit VIIc of cytochrome c oxidase (EC 1.9.3.1); (Acc# U53328); Arp2/3 protein complex subunit p34-Arc (ARC34) (Acc# AF006085); D53 (hD53) (Acc# M77830); Desmin (Acc# AF006012); Dynamin (DNM) (Acc# S72422); H5; and platelet glycoprotein Ib beta chain (Acc# U59632); Keratin, keratin 16 homolog (Acc# S72493); Lymph node homing receptor (Acc# M25280); Lysosomal-associated multitransmembrane protein (LAPTm5) (Acc# U51240); MHC protein homologous to chicken B complex protein (Acc# M24194); mRNA for Clock (Acc# AB005535); mRNA for lymphocte activation marker Blast-1 (Acc# X06341); mRNA for ORF (Acc# X80822); Myosin alkali light chain (ventricular) (Acc# M24122); Myosin light chain 3 non-muscle (MLC3nm) (Acc# M31212); N-acetylglucosaminyltransferase I (GlcNAc-TI) (Acc# M55621); Nicotinic acetylcholine receptor alpha3 subunit precursor, (Acc# U62432); p167 (Acc# U58046); P2x1 receptor (Acc# U45448); Partial C1 mRNA (Acc# X78817); Protein-tyrosine kinase (JAK1) (Acc# M64174); rab2 mRNA, YPT1-related and member of ras family (Acc# X12953); Retinoid X receptor beta (RXR-beta) (Acc# M84820); Sorbitol dehydrogenase gene (Acc# U07361); Wilm's tumor-related protein (QM) (Acc# M64241), 2′,3′-cyclic nucleotide3′-phosphodiesterase (Acc# M19650), D53(hD53) (Acc# U44427), DNA fragmentation factor-45 (Acc# U91985), Drg1 (Acc# X92845), Epidermal growth factor receptor substrate (eps15) (Acc# U07707),Myosin heavy chain (Acc# M35230), ARL1 (Acc# L28997), Clone 22 mRNA, alternative splice variant beta-2 (AF009424), Glutathione-S-transferase homolog (Acc# U90313), Leukemia virus receptor 2 (Acc# L20852), Muscle specific enolase (Acc# X51957), Type 3 inositol 1,4,5-triphosphate receptor (D26351), Hrs (Acc# D84064); and
(d) comparing the pattern of gene expression from said sample with either or both of the following: (i) the pattern of gene expression in the same mammal or the same intestinal cells in the absence of the test compound; or (ii) in a library of known patterns of gene expression from normal cells;
wherein test compounds that cause the hybridization pattern to approach the pattern seen in normal cells are thereby identified as candidates for drugs to treat inflammatory bowel disease, Crohn's disease, or ulcerative colitis.

14. The method of claim 13, wherein the sample comprises blood cells from the patient.

15. The method of claim 13, wherein the gene expression is detected using DNA microarray hybridization.

16. The method of claim 15, wherein a oligonucleotide is isolated from the sample.

17. A method to assess a patient's risk of having, or developing inflammatory bowel disease, including Crohn's disease and ulcerative colitis, said method comprising the steps of:
(a) obtaining a sample of body fluid or tissue from the patient;
(b) detecting a pattern of gene expression present in said sample of genes selected from the group consisting of Acidic calponin (Acc# S80562), Beta-sarcoglycan A3b (Acc# U31116), CL100 mRNA for protein tyrosine phosphatase (Acc# X68277), ZIP-kinase (Acc# AB007144), G protein gamma-4 subunit (Acc# U31382), Fibroblast muscle-type tropomyosin (Acc# M12125), Alkali myosin light chain 1 (Acc# M20642), Achaete scute homologous protein (Acc# L08424), Sorting nexin 2 (SNX2) (Acc# AF043453), Tristetraproline (TTP) (Acc# M63625), serine/threonine protein kinase (#D86550) (Acc# U59305), KIAA0210 (Acc# D86965), Methionine aminopeptidase (Acc# U29607), (p23) (Acc# L24804), Placenta (Diff48) (Acc# U49187), Rac3 (RAC3) (Acc# AF008591), jun-B (Acc# X51345), tyrosine phosphatase (Acc# Z68092), Complement component C3 mRNA, alpha and beta subunits (Acc# K02765), PKC alpha (Acc# X52479), SHPS-1 (Acc# D86043), Hbrm (Acc# X72889), maleylacetoacetate isomerase(Acc# AJ001838), mRNA encoding GPI-anchored protein p137 (Acc4 Z48042), Homolog of Drosophila enhancer of split m9/m10 (Acc# U04241), (p23) (Acc# L24804), (Acc# D21267), 14-3-3 protein epsilon isoform (Acc# U43399); 47 kD autosomal chronic granulomatous disease protein (Acc# M55067); Adaptor protein XII beta (Acc# AF047348); Adenylate kinase 2 (adk2) (Acc# U39945); Adenylyl cyclase-associated protein (CAP) (Acc# L12168); alpha-CP1 (Acc# U24223); Apoptotic protease activating factor 1 (Apaf-1) (Acc# AF013263); cAMP-dependent protein kinase subunit RII-beta (Acc# M31158); COX VIIc gene for subunit VIIc of cytochrome c oxidase (EC 1.9.3.1); (Acc# U53328); Arp2/3 protein complex subunit p34-Arc (ARC34) (Acc# AF006085); D53 (hD53) (Acc# M77830); Desmin (Acc# AF006012); Dynamin (DNM) (Acc# S72422); H5; and platelet glycoprotein Ib beta chain (Acc# U59632); Keratin, keratin 16 homolog (Acc# S72493); Lymph node homing receptor (Acc# M25280); Lysosomal-associated multitransmembrane protein (LAPTm5) (Acc# U51240); MHC protein homologous to chicken B complex protein (Acc# M24194); mRNA for Clock (Acc# AB005535); mRNA for lymphocte activation marker Blast-1 (Acc# X06341); mRNA for ORF (Acc# X80822); Myosin alkali light chain (ventricular) (Acc# M24122); Myosin light chain 3 non-muscle (MLC3nm) (Acc# M31212); N-acetylglucosaminyltransferase I (GlcNAc-TI) (Acc# M55621); Nicotinic acetylcholine receptor alpha3 subunit precursor, (Acc# U62432); p167 (Acc# U58046); P2x1 receptor (Ace# U45448); Partial C1 mRNA (Acc# X78817); Protein-tyrosine kinase (JAK1) (Acc# M64174); rab2 mRNA, YPT1-related and member of ras family (Acc# X12953); Retinoid X receptor beta (RXR-beta) (Acc# M84820); Sorbitol dehydrogenase gene (Acc# U07361); Wilm's tumor-related protein (QM) (Acc# M64241), 2′,3′-cyclic nucleotide3′-phosphodiesterase (Acc# M19650), D53(hD53) (Acc# U44427), DNA fragmentation factor-45 (Acc# U91985), Drg1 (Acc# X92845), Epidermal growth factor receptor substrate (eps15) (Acc# U07707),Myosin heavy chain (Acc# M35230), ARL1 (Acc# L28997), Clone 22 mRNA, alternative splice variant beta-2 (AF009424), Glutathione-S-transferase homolog (Acc# U90313), Leukemia virus receptor 2 (Acc# L20852), Muscle specific enolase (Acc# X51957), Type 3 inositol 1,4,5-triphosphate receptor (D26351), Hrs (Acc# D84064); and
(c) comparing the pattern of gene expression from said sample with a library of known patterns of gene expression from patients with inflammatory bowel disease, Crohn's disease and ulcerative colitis; and
(d) assessing the risk of the patient for inflammatory bowel disease, Crohn's disease, or ulcerative colitis as indicated by the number of genes that are overexpressed or underexpressed in the sample as compared with the patterns of gene expression from the library.

18. The method of claim 17, wherein the sample comprises blood cells from the patient.

19. The method of claim 17, wherein the gene expression is detected using DNA microarray hybridization.

20. The method of claim 19, wherein a oligonucleotide is isolated from the sample.

21. A drug screening assay comprising:
(a) administering a test compound to a mammal having an inflammatory bowel disease, including Crohn's disease or ulcerative colitis, or to intestinal cells isolated from a mammal with inflammatory bowel disease, including Crohn's disease or ulcerative colitis;
(b) obtaining a sample from the mammal or the cell composition;
(c) detecting a pattern of gene expression present in said sample of genes selected from the group consisting of the genes listed in Tables 1, 2, 3, 4, and 5; and
(d) comparing the pattern of gene expression from said sample with either or both of the following: (i) the pattern of gene expression in the same mammal or the same intestinal cells in the absence of the test compound; or (ii) in a library of known patterns of gene expression from normal cells;
wherein test compounds that cause the hybridization pattern to approach the pattern seen in normal cells are thereby identified as candidates for drugs to treat inflammatory bowel disease, Crohn's disease, or ulcerative colitis.

22. A method to assess a patient's risk of having, or developing inflammatory bowel disease, including Crohn's disease and ulcerative colitis, said method comprising the steps of:
(a) obtaining a sample of body fluid or tissue from the patient;
(b) detecting a pattern of gene expression present in said sample of genes selected from the group consisting of the genes listed in Tables 1, 2, 3, 4, and 5; and
(c) comparing the pattern of gene expression from said sample with a library of known patterns of gene expression from patients with inflammatory bowel disease, Crohn's disease and ulcerative colitis; and
(d) assessing the risk of the patient for inflammatory bowel disease, Crohn's disease, or ulcerative colitis as indicated by the number of genes that are overexpressed or underexpressed in the sample as compared with the patterns of gene expression from the library.

23. A method to diagnose inflammatory bowel disease in a human patient, said method comprising the steps of:
(a) obtaining a sample of body fluid or tissue from the patient;
(b) detecting a pattern of gene expression present in said sample of genes selected from the group consisting of the genes listed in Table 3; and
(c) comparing the pattern of gene expression from said sample and one or more entries from a library of known patterns of gene expression from patients with inflammatory bowel disease.

24. A method to diagnose Crohn's disease in a human patient, said method comprising the steps of:
(a) obtaining a sample of body fluid or tissue from the patient;
(b) detecting a pattern of gene expression present in said sample of genes selected from the group consisting of the genes listed in Tables 1, 3, and 4; and
(c) comparing the pattern of gene expression from said sample and one or more entries from a library of known patterns of gene expression from patients with Crohn's Disease.

25. A method to diagnose ulcerative colitis in a human patient, said method comprising the steps of:
(a) obtaining a sample of body fluid or tissue from the patient;
(b) detecting a pattern of gene expression present in said sample of genes selected from the group consisting of the genes listed in Tables 2, 3, and 5; and
(c) comparing the pattern of gene expression from said sample and one or more entries from a library of known patterns of gene expression from patients with inflammatory bowel disease.
